(19) United States
(12) Patent Application Publication
(10) Pub. No.: US 2010/0323343 A1
(43) Pub. Date: Dec. 23, 2010

(54) METHODS AND COMPOSITIONS FOR ANALYTE DETECTION

(75) Inventors: Richard Laswell Egan, Oceanside, CA (US); Graham Peter Lidgard, La Jolla, CA (US); David Dickson Booker, Oceanside, CA (US); Christopher Johann Johnson, San Diego, CA (US); Alexander Belenky, San Diego, CA (US); Stan Vukajlovich, San Diego, CA (US); John Zeis, Vista, CA (US); Scott Castanon, Vista, CA (US)

Correspondence Address: COOLEY LLP;ATTN: Patent Group, Suite 1100, 777 - 6th Street, NW, WASHINGTON, DC, 20001, US

(73) Assignee: Nexus DX, Inc., San Diego, CA (US)

(21) Appl. No.: 12/777,969

(22) Filed: May 11, 2010

Related U.S. Application Data
(60) Provisional application No. 61/177,272, filed on May 11, 2009.
(60) Provisional application No. 61/228,135, filed on Jul. 23, 2009.

(51) Int. Cl. C12Q 1/70; G01N 30/00; C12M 1/34; G01N 33/566

(52) U.S. Cl. 435/5; 422/69; 422/430; 435/287.3; 436/501

(57) ABSTRACT

The present invention is directed to methods and apparatus for detection of one or more analytes. Analytes include agents or components of infectious agents such as pathogenic virus, as well as enzymes, proteins and biomarkers.

### PRIORITY

This application claims priority to U.S. Provisional Application No. 61/177,272 filed May 11, 2009, and to U.S. Provisional Application No. 61/228,135 filed Jul. 23, 2009, each of which is hereby incorporated by reference in its entirety.

### STATEMENT AS TO GOVERNMENT SUPPORTED RESEARCH

Portions of this invention may have been made with the support of the United States government under contract number 200-2007-19345 granted by the Center for Disease Control. The Government may have certain rights to portions of this invention.

### BACKGROUND OF THE INVENTION

This invention relates to assays for analyte(s), e.g., antigens, in a sample such as a biological sample obtained from a subject. In particular, the invention relates to method(s) and device(s) for the detection of one or more analytes utilizing binding moieties specifically targeting a selected analyte. The analytes may be, for example, one or more infectious agents.

Many types of assays have been used to detect the presence of various substances in bodily samples, often generally called analytes or ligands. These assays typically involve antigen-antibody reactions (e.g., ligand, anti-ligand, ligand-receptor) and can utilize synthetic conjugates comprising radioactive, enzymatic, fluorescent, or visually observable metal soluble tags, and specially designed reactor chambers for observing results. Most current tests are designed to make a quantitative determination, but in many circumstances all that is required is a qualitative identification, e.g., positive/negative indication.

Qualitative assays must be very sensitive because of the often small concentration of the analyte of interest in the test fluid. Further, false positives can be troublesome, particularly with agglutination and other rapid detection methods such as dipstick and color change tests. Sandwich immunoassays and other detection methods which use metal soluble tag or other types of colored particles have been developed. Such techniques still suffer from problems encountered in rapid detection methods designed to detect a plurality of target analytes. Moreover, with the emergence of highly pathogenic agents such as influenza virus, there is a need to develop effective laboratory or point-of-care systems that can effectively and accurately detect one or more infectious agents, including different types or subtypes of an infectious agent.

For example, influenza is commonly seen in local outbreaks or epidemics throughout the world. Epidemics may appear at any time and can occur explosively with little or no warning. The number of people affected can vary from a few hundred to hundreds of thousands to millions. Epidemics may be short-lived, lasting days or weeks, but larger epidemics may last for months. Although influenza is typically mild in most individuals, it is life threatening to elderly, the very young or debilitated individuals. However, certain strains of flu, such as H1N1 and H5, have been shown to be lethal even in healthy and young individuals. Therefore, there is a need to develop devices and methods to effectively detect one or more types and subtypes of a pathogen, such as influenza, whether the infection is caused by a typical or expected subtype of influenza (seasonal flu) or a subtype that can be the causative agent of an epidemic or pandemic (e.g., bird flu or swine flu).

It is an object of this invention to provide a rapid and sensitive method for detecting analytes in a biological sample. Another object is to provide an assay which has high sensitivity and fewer false positives than conventional assays. A further object is to provide an apparatus or system for detection of low levels of analytes present in biological samples. Another object is to provide an assay system that involves a minimal number of procedural steps, and yields reliable results even when used by persons in the absence of special training.

One object of the invention is to provide a system for testing infectious agents that provides results identifying one or more infectious agents in a matter of minutes.

A further object provides a system where results on a testing implement are equally specific and sensitive for the target analytes, notwithstanding that results can be read one to several hours after completion of a reaction necessary to obtain a result. These and other objects and features of the invention will be apparent from the following description, drawings, and claims.

### SUMMARY OF THE INVENTION

In one aspect of the invention, a sample collection device is provided that is configured to allow mixing a sample in a solution, where the solution comprises the reagents necessary to detect one or more target analytes. The sample collection device may be configured to allow for an air-tight seal between a sample receiving tube component and a upper-sealed chamber component of the sample collection device, whereby the receiving tube and upper sealed chamber are capable of being pressure-fit together to provide positive back pressure that helps release a fluid contained in the sample collection device, when the sample collection device is coupled to a test device.

In another aspect, the invention provides a test device that comprises a lateral flow membrane, a chamber comprising fluid upstream of the direction of lateral flow, wherein the chamber is capable of controllably releasing the fluid into the lateral flow material. The device includes a plurality of addressable lines comprising one or more test zones and one or more control regions; and a plurality of capture moiety partners disposed in each of the addressable lines. In one embodiment, the test device comprises a test strip. The test strip comprises at least two adjacent addressable lines having a different category of capture moiety partner immobilized thereto. In one embodiment, each addressable line is configured to detect a different target analyte.

In another aspect, a method is provided for detecting one or more target analytes comprising mixing a sample with reagents in a sample collection device to form a complex, where the complex comprises a capture probe, a target analyte, and a detection probe, and wherein the complex is released from the sample collection device to a test device through a split-septum present at the distal end of the sample collection device. The complex is allowed to run through a test device comprising a test strip having a plurality of addressable lines, wherein each of the addressable lines is configured to detect a different analyte, and wherein each addressable line of the test strip comprises a population of one type of immobilized capture moiety partner that is complementary to a capture moiety present in the sample collection device. In a further embodiment, the test device comprises a test strip with one or more control lines.

In yet another aspect, the invention provides a system for detecting an anlyate comprising a sample collection device and a test device.

In another aspect, the invention provides a kit, which comprises a test device and a plurality of specific binding reagents.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages will be obtained by reference to the following detailed description that sets forth illustrative embodiments, in which the principles of the invention are utilized, and the accompanying drawings of which:

FIG. 1 illustrates a sample collection device.

FIG. 2 illustrates a sample collection device: 2A illustrates a sample collection device with a blow up view of an upper chamber and sample assembly; 2B illustrates a sampling assembly.

FIG. 3 illustrates one embodiment of a sample collection device disassembled.

FIG. 4 illustrates one embodiment of a sample collection device.

FIG. 5A-C illustrates assembly indicators on the sample collection device.

FIG. 6 illustrates a split septum on a sample collection device.

FIG. 7 illustrates a schematic of an outlet region of the sample collection device.

FIG. 8 illustrates a schematic of a dispensing tip of an outlet region of a sample collection device.

FIG. 9 illustrates a schematic of the outlet region of a sample collection device.

FIG. 10 illustrates a schematic of an interface between an outlet region of a sample collection device and a port of a test device.

FIG. 11 illustrates a schematic sample collection device coupled to a test device.

FIG. 12 illustrates one embodiment of a diagnostic assay system including a sample collection device and a test device.

FIG. 13 illustrates a test device.

FIG. 14 illustrates a schematic of a test device comprising a cannula to receive a septum sample collection device.

FIG. 15 depicts a blow up illustration of a test device having a cannula to receive a septum sample collection device.

FIG. 16 illustrates a test device.

FIG. 17 illustrates a schematic of a test device.

FIG. 18 illustrates a schematic of pRNA binding of multiple analytes on a test strip.

FIG. 19 illustrates a lateral flow test.

FIG. 20 illustrates an anchor molecule phenylene diisothiocyanate (PDITC) linked to a 12-carbon spacer.

### DETAILED DESCRIPTION OF THE INVENTION

Various aspects of the invention are directed to devices and binding pair assays that utilize specific binding moieties and capture moieties for the qualitative and/or quantitative analysis of selected analytes in samples. The invention is useful in a variety of assays for detection of one or more infectious agents that may be present in a sample. Assays useful in the invention include, but are not limited to, competitive immunoassays, non-competitive immunoassays, sandwich immunoassays and blocking assays.

In one embodiment, a sample collection device (SCD) is utilized to collect a sample and/or process a sample with immunoreactive reagents that provide a detection means and a capture means. The sample containing one or more analytes is mixed in a SCD to form a mixture that can be stored or reacted with specific binding reagents in the SCD and subsequently expelled to a test device (TD) that provides immobilized reagents that capture analyte complexes in the sample. The specific binding reagents in the SCD comprise detectable labels, signals, or indicators as further described herein that can be read by the naked eye or with an instrument. Furthermore, the test device can be configured to allow detection of multiple analytes. Such analytes can be from one or more infectious agents, including different strains and/or subtypes of an infectious agent. Detection can include qualitative and/or quantitative measurements of one or more analytes.

In various embodiments, the plurality of specific binding agents to detect the analytes comprise a plurality of Analyte Binding Sets, wherein each set comprises specific binding agents that bind one target analyte (e.g., antigen). In some embodiments, multiple Analyte Binding Sets are included that provide second and subsequent groups of specific binding pairs which specifically bind a second, third, fourth, fifth or more different analytes (e.g., antigens from different infectious agent or subtypes of an infectious agent). In one embodiment, an SCD can comprise two, three or four different groups of Analyte Binding Sets wherein each Set is configured to detect different type or subtypes of influenza virus antigens.

In various embodiments a particular Analyte Binding Set comprises reagents necessary to bind a particular target analyte for which the particular set is configured. In various embodiments, each Analyte Binding Set comprises: (1) a capture probe and (2) a label probe, with each Analyte Binding Set designed to specifically bind a different analyte.

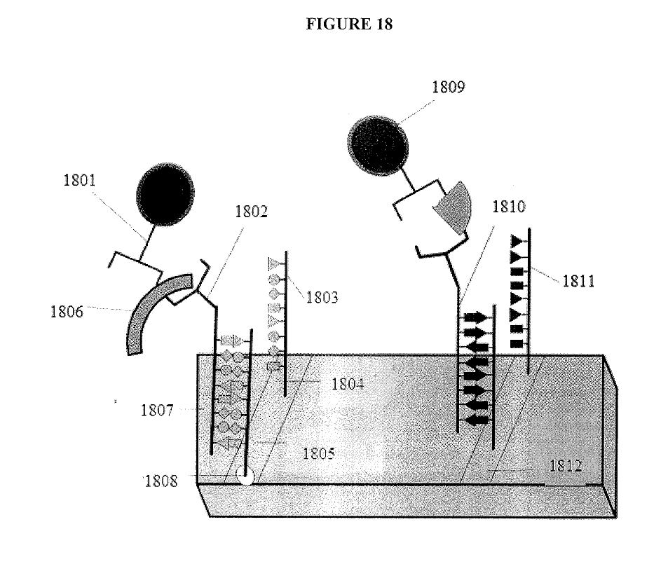

A capture probe (e.g., 1802 in FIG. 18) comprises: (i) a specific binding agent that binds (directly or indirectly) to a specific analyte; and (ii) a capture moiety partner (e.g., 1807). A detection probe 1801 comprises: (i) a specific binding agent that binds (directly or indirectly) to the same specific analyte as the capture probe; and (ii) a label 1809. Labels that can be used are disclosed herein and include, for example, europium labels. The sample containing one or more analytes is reacted in the SCD with one or more Analyte Binding Sets to form a complex of the capture probe, analye and detection probe. These complexes of different target analytes when present are captured on different addressable lines (e.g., 1805 and 1812 by an immobilized capture moiety partner 1803, 1811).

In one embodiment, a capture probe comprises a target antibody that is linked, directly or indirectly, to a capture moiety partner. The capture moiety partner is “captured” by a cognate immobilized capture moiety partner disposed on the solid support (e.g., nitrocellulose membrane) as an addressable line in the Test Device. Such capture moieties are referred to herein as Capture Moiety Partners (CMP(s)). A CMP as used herein means a molecule that specifically binds with a second capture moiety partner. For example, a CMP can comprise a first pRNA molecule of a particular sequence, and that binds to a second pRNA molecule (capture moiety partner) complementary to the first molecule, allowing specific binding of the two molecules when they come into contact with each other.

In various embodiments, a CMP comprises molecules including but not limited to pRNA or pDNA molecules, an aptamer and its cognate target, or streptavidin-biotin, or other ligand/receptor pair. For a given set of CMPs, the two molecules are related in the sense that their binding with each other is such that they are capable of distinguishing their binding partner from other assay constituents having similar characteristics.

In various embodiments, a detection probe and capture probe comprise analyte-specific binding agents that include but are not limited to an antibody or functional fragment thereof.

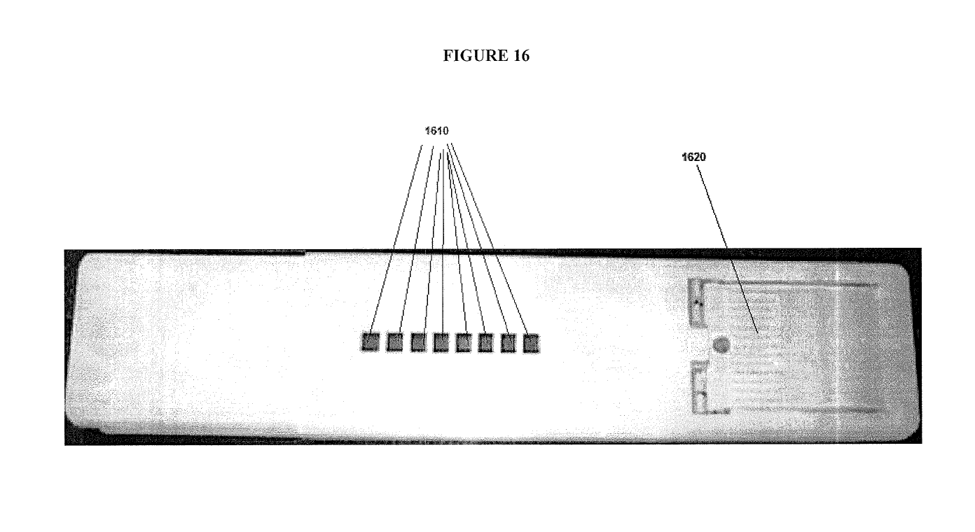

In one embodiment, for each capture moiety partner present in a conjugate capture probe, a cognate capture moiety partner is immobilized (“ICMP” for Immobilized Capture Moiety Partner) in a discrete position (addressable line) viewable on a test membrane present in a test device (e.g., FIG. 16). As used herein the term “immobilized” in the context of an ICMP means that the ICMP is not mobilizable, regardless if a solution is processed through a test strip comprising the ICMP.

An ICMP is positioned on a test membrane present in a TD, wherein ICMPs (e.g., 1803, 1811) immobilized on an addressable line are capable of specifically binding their cognate capture moiety partner (i.e., present in a capture probe). For example, if an ICMP is a pRNA molecule, it will specifically bind its cognate capture moiety partner present on a capture probe 1801 (i.e., antibody specific for a target antigen conjugated with the cognate capture moiety partner) so that if an analyte-capture probe complex is formed, such a complex is “captured” by the ICMP on a specified addressable line. As used herein the term “addressable line” includes lines, spots or any other region that is discrete and positioned in a different region of a test strip as compared to any other addressable line, wherein different addressable lines are configured to detect different analytes by virtue of having different pairs of CMP(s) in the detection probe and immobilized on the test device.

In various embodiments, a CMP and ICMP configured for one target analyte are selected from any two molecules that specifically bind to each other, and such molecules include but are not limited to oligonucleotides, avidin and streptavidin, pyranosyl RNAs (pRNAs), pyranosyl DNAs (pDNAs), an aptamer and its binding partner, or any ligand and its binding partner.

In one embodiment, a test device comprises a membrane, and the membrane comprises at least two addressable lines adjacent to each other that have a different type of capture moiety partner. It should be understood that “different type” as used in the context of two adjacent addressable lines means a different type or class of chemical or physical entity as opposed to the same type of chemical or physical entity having different binding specificity. In one embodiment, a membrane has different addressable lines that are configured to detect multiple different analytes, and the addressable lines can have the same type or class of immobilized capture moiety partner or alternatively, in another embodiment, the addressable lines can have different types of capture moiety partner, but in both cases the addressable lines are configured to each detect a different analyte. In one embodiment, by selecting a different type or class of capture moiety partners for each of two adjacent addressable lines, the invention provide an assay that eliminates or substantially reduces cross-reactivity between capture moiety partners between different addressable lines. Therefore, the overall performance of an assay for multiple analytes using devices of the invention is improved, by increasing specificity and/or sensitivity (e.g., Examples 1-3).

In some embodiments, the CMPs are selected from the same type or class of molecule. For example, the CMPs can have different pairs of capture probe and ICMP, of which each are oligonucleotides (e.g., pRNAs or pDNAs), but have different binding pair specificity so each pair is configured to identify a different analyte. In other embodiments, the CMP pairs are selected from different types of molecules and additionally are configured to identify different analytes. For example, pRNA is utilized for the CMP pair for one specific analyte, while a different type of capture moiety partner (e.g., streptavidin) for another analyte, and different specific binding partners, such as an antigen and antibody, are used as a third CMP pair. In some embodiments, two or more different types or classes of capture moiety partners are used in a SCD and TD of the invention (e.g., two, three, four or more different types).

In some embodiments, the different analytes detected are viruses or components of viruses (e.g., polypeptides). In various embodiments, the different antigens are from influenza viruses and/or subtypes of influenza virus. In one embodiment, the influenza virus that can be detected is influenza A virus and/or influenza B virus, as well as subtypes of influenza virus A and/or B. One embodiment is directed to detection of influenza A and B and subtypes of the formula HxNy, wherein x can be 1-16 and y can be 1-9, or any combination of xy thereof.

In yet other embodiments, the different analytes detected are one or more different infectious agents and/or one or more different subtypes of an infectious agent, including but not limited to HIV, HCV, HPV, HSV, a bacterium (e.g., myobacterium such as tuberculosis), or fungi (e.g., yeast), or a combination thereof.

In various embodiments, a SCD comprises a sampling implement that provides a means to collect a sample from a subject. The sampling implement may be coupled (permanently or removably) to an upper chamber via a sampling implement holder. The sampling implement can be disposed at the distal end of a shaft, wherein the shaft can be solid, hollow or semi-permeable. In some embodiments, the sampling implement is a swab, a comb, a brush, a spatula, a rod, a foam material, a flocculated substrate or a spun substrate.

In various embodiments, an SCD comprises one or more sealed chambers, wherein the seal functions to preclude fluid communication between a second chamber of the SCD. In some embodiments, the seal comprises a break-away valve, a flapper valve, a twist valve, screw valve, rupturable seal, puncturable seal or breakable valve.

In further embodiments, opening a seal can allow the contents of an upper chamber to flow through to a lower chamber(s) of the sample receiving tube. In other embodiments, the upper chamber can contain one or more ampoules which prevent solutions contained therein to flow to the lower chamber, unless pressure is exerted to rupture, puncture or break the ampoule so as to release contents therein.

In another embodiment, a TD is provided for detection of one or more analytes, wherein the device comprises a lateral flow membrane in a body, a chamber upstream of the lateral flow membrane containing a fluid or solution, wherein a gap is disposed between said chamber and said lateral flow membrane thus precluding fluid communication between the chamber and the lateral flow membrane. In one embodiment, the pressure exerted on the chamber pushes the gap closed thus forming fluid communication between the chamber and the lateral flow membrane. In one embodiment, an opening into which a distal end of an SCD fits, is disposed directly above a wicking pad that is disposed downstream of the gap, but upstream of the lateral flow membrane.

In one embodiment, the Test Device chamber comprises one or more subchambers containing the same or different solutions. In other embodiments, the chamber or subchambers comprise one or more ampoules that are breakable, puncturable or rupturable. Thus, where pressure is exerted on such ampoules the contents are controllably released. As described herein, a Test Device may or may not comprise a gap means for disrupting fluid communication from the chamber to the lateral flow membrane. A Test Device gap can be from zero to 3.0, 0.5 to 3.5, 1.0 to 2.5, 1.0 to 3.0, or 2.0 to 4.0 mm.

In some embodiments, a Test Device can comprise a body housing the lateral flow membrane, wherein the body provides one or a plurality of windows 1610 through which the lateral flow membrane is visible. In various embodiments described herein, a TD comprises a lateral flow membrane that comprises a wicking substrate and an absorbent substrate upstream or downstream of the test zones disposed on said lateral flow membrane. In some embodiments, a substrate for collecting a small volume of sample for archiving is provided in a SCD or Test Device. In one embodiment, the substrate providing such archiving means is a filter, membrane or paper that collects a small volume of sample and said substrate is subsequently removed from the device.

In various embodiments, a SCD and/or a TD comprises one or more identical identifiable tags, which can be removed from one device and placed on another device.

In some embodiments, the Test Device is shaped to fit (specialized adaptor shape) into the receiving port of a reader when the upstream chamber has been depressed thus indicating that wash buffer or chase buffer contained therein has been released through the lateral flow membrane. In such embodiments, a specialized adaptor present in the Test Device and Reader provides a means to verify that chase buffer or solution in the upstream chamber of the Test Device has been released and thus indicates that any sample present upstream of the lateral flow membrane is washed through the lateral flow membrane. Thereby, the specialized adaptor provides a “safety means” to prevent reading of unprocessed samples.

In another aspect of the invention, the processed samples are run through the Test Device's lateral flow membrane, but can be placed aside from 30 minutes to several hours. In various embodiments, a plurality of samples can be run through the Test Device but read at about 0.5, 1, 2, 3, 4, 5, 6, 7, 8, 9, 10, 11 or 12 hours later, with consistent and accurate signals.

In certain aspects of the invention, the devices disclosed herein are utilized in methods for detection of one or more analyte that may be present in a sample. In some embodiments, methods are directed to detecting one or more strains of an infectious agent. In one embodiment, a method is directed to utilizing the devices of the invention to detect one or more influenza viruses and/or subtypes thereof. For example, methods are provided for detection of influenza A virus and influenza B virus, and subtypes of influenza A that may be present in a single sample.

In one embodiment, a method is provided for determining whether a subject is infected with a pandemic strain of influenza virus, non-pandemic strain of influenza virus, or strain of influenza virus for which vaccine is available.

In some embodiments, the Test Device excludes any reagent or binding agent that is capable of specifically binding a target antigen, per se. The test device includes a CMP that is designed to indirectly capture the target analyte by specifically binding to the cognate CMP in the complex of analyte, capture probe and detection probe.

In one aspect of the invention, a reader is provided to detect a signal from a Test Device as an indication of the presence/absence of analyte(s), such as for example, a UV LED reader. In various embodiments, the signal detected is a fluorescence signal from a label molecule. In further embodiments, the label molecule is a lanthanide. In yet a further embodiment, the lanthanide is europium. In one embodiment, the reader comprises a UV photodiode. In another embodiment, the reader comprises a UV laser diode.

In some embodiments, the plurality of sets of Analyte Binding Sets provided in the SCD can contain one category of label (e.g., where each detection probe includes the same fluorophores or different fluorophores having different wavelength signals). In other embodiments, each detection probe may include in the conjugate a label selected from various different categories of labels (e.g., a combination of metals and fluorophores). Each detection probe may have the same or different label and they may come from the same or different category. In one embodiment, the capture moiety is an oligonucleotide such as pRNA or pDNA and the label is Europium.

In another aspect of the invention, a reader is configured to comprise at least one hard or permanent standard. In another embodiment, a reader is configured to comprise at least two or more hard standards. In various embodiments, a hard standard comprises a label molecule emitting a detectable signal. In further embodiments, the label is a fluorescence label. In another embodiment, the fluorescence label is a lanthanide. In yet a further embodiment, the lanthanide is Europium.

In another aspect of the invention, an SCD and Test device of the invention are used in a method to detect one or more analytes, wherein such an analyte is associated with a disease, pathologic or other physiological condition. In various embodiments, such analytes are biomarkers associated with a condition related to any body tissue, including but not limited to the heart, liver, kidney, intestine, brain, fetal tissue, or pancreas. In one embodiment, such analytes are associated with a cardiac condition (e.g., myocardial infarction).

In various embodiments, the devices of the invention can be utilized in any method to detect analytes, e.g., an antigen or protein in a sample obtained from a subject. In some instances, a method or device of the present invention can be used to detect any such analytes, through utilization of a particular panel of immunoreactive or specific binding reagents that are specific for the desired analytes.

In several aspects of the invention, the Test Device comprises an upstream chamber that contains a means for providing a wash/running buffer or liquid. In various embodiments, such a buffer or liquid comprises additional agents such as signal/detector molecules (e.g., detection substrates) that interact with the label in the detection probe and can be read by an optical reader or by direct visualization. In certain embodiments, the buffer or liquid is present in a compartment comprised of a glass ampoule, membrane pouch, sac, or form filled pouch. In further embodiments, such compartments are ruptured, broken or otherwise disrupted leading to release of their contents for example by exerting pressure on said compartments. In other embodiments, such compartments are punctured or lanced by an appendage or needle. In yet further embodiments, such compartments are protected by a safeguard means that precludes accidental or unintentional release of their contents.

Sample Collection Device. One aspect of the invention is directed to a sample collection device (“SCD”) that comprises the necessary means to collect a biological sample, as well as the reagents and buffers necessary to process and react with analytes in the sample so as to form complexes comprised of the specific binding reagents with their specific target analytes (e.g., multiple groups of Analyte Binding Sets of detection probes and capture probes forming complexes with multiple different target analytes when present in a sample).

In one embodiment, if a particular analyte is present, it will be bound by a detection probe and capture probe (e.g., an analyte having bound to both in the sample from the SCD); the capture probe in the complex in turn will bind to its cognate immobilized partner capture moiety on defined spots or addressable lines on the test strip (as described herein).

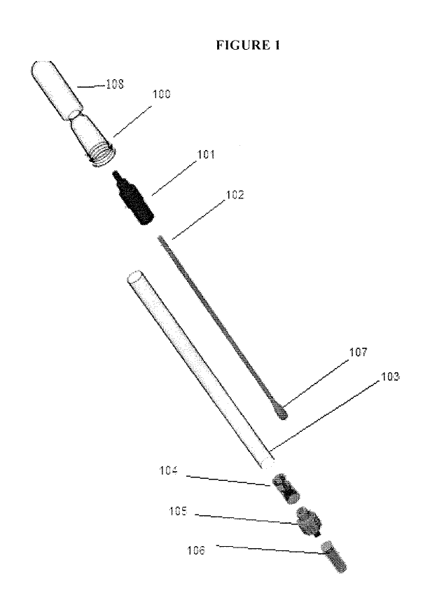

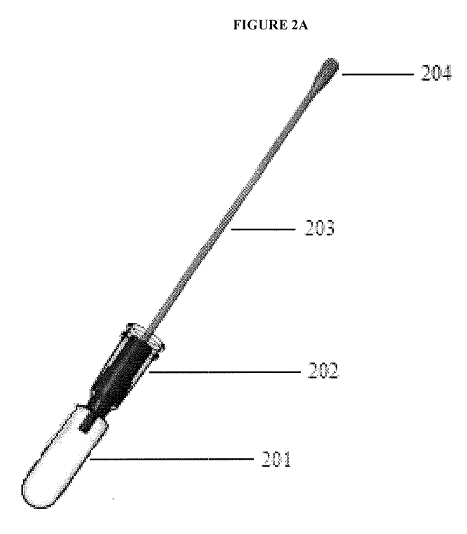

In one embodiment shown in FIG. 1, a SCD comprises an upper chamber component 100. The upper chamber component 100 can comprise one or more compartments. In some embodiments, the upper chamber 100 is comprised of a semi-rigid or depressible material. In other embodiments, the upper chamber 100 is comprised of a hard or rigid material. Materials useful for creation of a hard or rigid upper chamber 100 include, for example, hard plastics or glass. The one or more compartments present in an upper chamber can contain a solution, e.g., wash buffer, extraction buffer, reagent solution or a combination thereof.

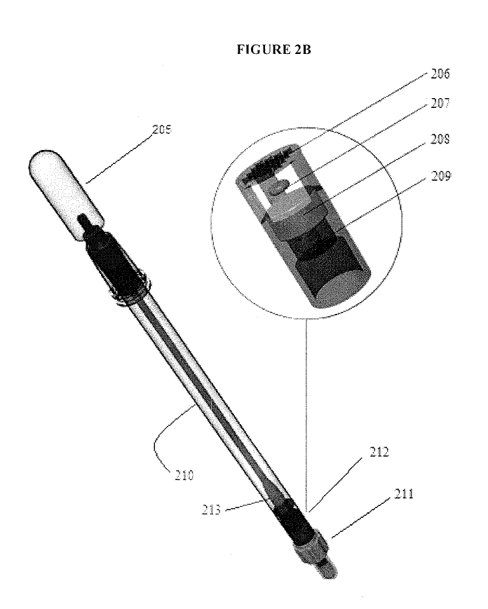

In one embodiment, a sample collection device (e.g., FIG. 1 and FIG. 2) comprises components that are fit together to produce negative back pressure that allows a solution to be released from the SCD in a uniform manner without a need for external pressure or manipulation of the SCD. In one embodiment, seating components of the upper chamber and a sample receiving tube 103, 210 are made of a hard or rigid material so that the two components can form a air-tight seal through force (e.g., force-fit). In a further embodiment, the coupling of the sample collection implement with the sample receiving tube through force-fit produces back pressure in the sample receiving tube that can expel any solution mixture from the distal end 106, 211 of the SCD when the SCD is coupled to a TD. In one embodiment, a SCD and TD are coupled via an orifice (e.g., split septum).

In one embodiment, a sample collection implement (e.g., collectively 100, 101, 102, 107 and 108; or also FIG. 2A) comprises at least one compartment 108, 201 that is positioned at the proximal end of the sample collection implement or upstream of the tube or stem 102, 203.

In a further embodiment, the compartment 108 is a sealed compartment of the upper chamber. In some embodiments, the solution in the upper sealed compartment is a buffer solution. In various embodiments, the volume of a solution present in or added to the upper chamber is about 10-500 μl or about 10 μl, 20 μl, 30 μl, 40 μl, 50 μl, 60 μl, 70 μl, 80 μl, 90 μl, 100 μl, 110 μl, 120 μl, 130 μl, 140 μl, 150 μl, 160 μl, 170 μl, 180 μl, 190 μl, 200 μl, 210 μl, 220 μl, 230 μl, 240 μl, 250 μl, 260 μl, 270 μl, 280 μl, 290 μl, 300 μl, 310 μl, 320 μl, 330 μl, 340 μl, 350 μl, 360 μl, 370 μl, 380 μl, 390 μl, 400 μl, 410 μl, 420 μl, 430 μl, 440 μl, 450 μl, 460 μl, 470 μl, 480 μl, 490 μl or 500 μl. In one embodiment, the solution volume is up to 150 μl. In another embodiment, the solution volume is up to 200 μl. In some embodiments, the solution in the upper chamber 100 is in a sealed compartment. The seal can be punctured, broken or opened via a valve structure, so as to provide fluid communication between the upper chamber 100 and stem 102 of the sampling assembly or the sample collection implement.

In one embodiment, a sealed chamber of the upper chamber can be a squeezable bulb that is capable of being compressed (e.g., user applies pressure to the bulb), thus controlling the flow rate of the solution (e.g., buffer) to the sampling implement. In some embodiments, the upper chamber is comprised of a bulb component that is a self-contained compartment that includes a solution. Such solutions include extraction, lysis, reagent, buffer or preservative solutions. In one embodiment, the solution is a buffer solution that is utilized to transfer the biological sample from the sampling implement down to the lower chamber.

The extraction solution should be of a sufficient volume to ensure wetting of any lyophilized assay reagents (e.g., lyophilized reagent beads) present and/or to extract the sample from the sample collection device. For example, where a dry swab is used as the sample swab, the volume of extraction solution sufficient for wetting the reagents and extracting or releasing the sample is 70 μl. In one embodiment, the extraction solution volume is at least 30 μl, 40 μl, 50 μl, 60 μl, 70 μl, 80 μl, 90 μl, 100 μl or greater. A person of ordinary skill in the art could easily determine a sufficient volume of extraction solution to ensure wetting of the dry swab sample and lyophilized reagent beads contained in the lower chamber which typically include the detection probe and capture probe.

An upper chamber can comprise one or more compartments. Each compartment can comprise a solution that is the same or different as solutions in other compartments. Such solutions can comprise reagents as desired including, but not limited to, extraction buffers, reducing agents, immunoreactive agents—such as anti-analyte specific binding agents comprising detection labels (e.g., detection probe)—and capture probe, if desired.

Reagents utilized in an SCD of the invention can include one or more salts, chelators, anticoagulants, detergents, stabilizers, diluents, buffering agents, enzymes, cofactors, specific binding members, labels, mucolytic and the like. It will be apparent to one of skill in the art that the particular reagents and/or combination of reagents can be tailored to the specific analyte(s) being assayed. The one or more reagents can be compounds that facilitate analysis of a sample. Furthermore, such reagents can readily be adapted for use in a Test Device of the invention.

A sample holder 101 can be in contact with the upper chamber component 100 and a sampling assembly. A sampling assembly can be removable from a housing comprising a sample receiving tube 103, and an upper chamber 100. In some embodiments, the sampling assembly has a stem 102 and a sample collection implement or substrate 107, which can function to facilitate sample collection (e.g. a swab). The length of the sampling assembly stem 102 can be optimized for sample collection, e.g., designed for a length to accommodate sample collection from different anatomical sites including, but not limited to the throat, mouth, nose, ear, urethra, anus and vagina. For example, the length of the device (e.g., integrated configuration) can be about 1 to 9 inches, or about 2, 3, 4, 5, 6, 7, 8 or 9 inches. The sampling assembly can be placed into the sample receiving tube 103 to provide an integrated configuration. In such a configuration, a sampling implement is upstream of and in fluid communication with the lower chamber mixing or reagent component 104 via the stem or tube 102.

In some embodiments, a sample collection implement includes a stem or tube 102 that is hollow, solid or semi-porous. In some embodiments where the stem or tube of the sampling assembly is porous or bibulous, the sampling assembly actually provides a path of fluid communication from the upper chamber component 100 to the sampling substrate (e.g., swab) 107. The sample collection implement (e.g., 100, 101, 102, 107 and 108) can be held by a sample holder 101 that can fit into a receiving end of the upper chamber 100.

In some embodiments, the stem or tube 102 present in a sample collection implement is a portion that extends into the upper chamber 100 and has a terminal end that is closed. In one embodiment, a portion of the terminal end of the stem or tube 102 is snapped or broken, thereby opening a fluid communication between the upper chamber component 100 down through the sampling assembly to a sampling substrate 107 (e.g., swab).

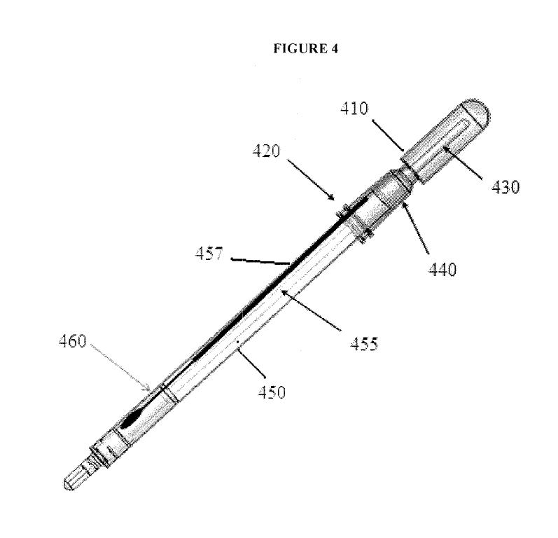

In another embodiment, a sample collection device comprises a stem or tube that provides a fluid communication between the upper chamber, but a sample is placed in the sample receiving tube using a separate component for collecting and holding the sample (e.g., as depicted in FIG. 4, 457).

The lower chamber mixing or reagent component 104 can contain reagents that specifically bind to one or more target antigens. The lower chamber mixing or reagent component 104 can comprise one or more compartments. For example, two compartments can be arranged in series in the lower chamber mixing or reagent component 104. The lower chamber mixing or reagent component 104 can be in contact with a luer 105 that can be in contact with a cap 106. The orientation of the SCD is such that the compartment 108 is at the proximal end and the cap 106 is at the distal end.

In one embodiment, a sample collection device is configured to swap out different lower chamber or mixing compartments (e.g., through snap fit, or screw threads of the SCD and lower chamber compartment), whereby the lower chamber compartment comprises the necessary reagents for a specific assay (e.g., detection of particular target analytes), while the upper chamber comprises wash buffer and/or extraction reagents. In another embodiment, the swappable lower chamber compartment comprises extraction reagents as well as reagents necessary to form an analyte-reagent complex as described herein.

In another embodiment, the distal end of the SCD is open, whereby prior to release of a solution from the upper sealed chamber, the SCD is engaged (e.g., by friction fit) into the receiving port of a TD. In such an embodiment, the fluid flow from the distal end of the SCD into the TD need not be regulated by a luer or a valve structure, but fluid flow can be obtained via, e.g., the creation of negative pressure within the TD or a differential pressure between the SCD and TD, gravity or capillary flow.

In another embodiment, the distal end of the SCD does not utilize a valve but rather is open. The SCD may be attached to the test device prior to release of the buffer from the upper chamber. Upon release of the solution from the upper chamber, the sample is released and/or extracted from the collection implement by the solution and mixed with the reagents located in the lower chamber. The mixture then flows to the test device for analysis of the presence of one or more analytes. It is possible to include water-dissolvable membranes within the lower chamber to slow the flow of the mixture out of the SCD onto the test device. Such membranes are conventional and can be designed to permit the retention of the mixture for differing periods of time sufficient to allow mixing and reaction of the reagents and sample analytes. For example, such membranes can be prepared from any of a variety of known proteins, polysaccharides or film formers.

In one embodiment, as shown in FIG. 2A, an SCD has an upper chamber component 201 to which is attached a sample holder 202 a sampling assembly tube or stem 203 and a sample collection implement 204.

In some embodiments, as illustrated in FIG. 2B, a liquid solution comprising the necessary reagents (e.g., detection/capture specific binding agents, etc.) can be disposed in the reagent area 208 of the lower chamber 212 (also shown in enlarged view) in liquid communication with the upper chamber component 205 via transport through the sample receiving tube 210. Fluid from the upper chamber 205 can flow down to the sample collection implement 213 to extract sample. The extracted sample can pass through an aperture 206 that may restrict/control the liquid flow from the upper chamber 205 to the lower chamber 212, comprising, for example, an aperture to control flow by size (e.g., size of perforations, type of substrate, or filter). The lower chamber 212 may contain a reagent area 208. In one embodiment, the reagent area 208 contains a solid reagent 207 that includes the necessary reagents (e.g., immunoassay reagents, such as detection and capture probes, etc.), formed as a dried solid, separately disposed or in a unified solid. The lower chamber can also include a filter 209 and at the distal end, there can be provided a luer 211.

In one embodiment, the upper chamber 330 comprises a valve 320 that allows controllable release of a solution in the upper chamber. The valve may be any type of valve known in the art and compatible with the system described herein. Additional valves that can be utilized include a rotary, breakable, stopcock, gate, ball, flapper, needle, butterfly, pinch, bellows, piston, slide, plug, diverter, or actuator valve. For instance, the valve may be a break-away valve, a snap valve, a flapper valve, a twist, screw, rupturable, puncturable or breakable valve. For example, where the valve is a snap-valve, the user applies force to the valve stem to break the stem, whereby the breakaway feature allows buffer to enter sample collection tube and the lower chamber via the stem. In one embodiment, the upper chamber is under positive pressure, such that opening of a valve or breaking of a seal results in an outflow of a solution in the upper chamber. In one embodiment, the upper chamber is under sufficient positive pressure such that the solution in the upper chamber flows under pressure to enter the lower chamber via the stem. For example, where the valve is a snap-valve, the user applies force to break the snap-valve stem, and the solution in the upper chamber flows under slight pressure entering the lower chamber via the stem. The upper chamber can be, for example, under 1, 10, 50, 100, 500, 1000, 5000, 10000, 20000, 30000, 40000, 50000 or more Pascal (Pa) of pressure. In one embodiment, the snap-valve has one stop. A snap-valve stem having one stop position is useful for preventing incomplete snapping, which could result in leakage of air into the upper chamber and incomplete delivery of the fluid.

Therefore, where a sample is washed downward via the solutions (e.g., buffer or wash solutions) provided in the upper chamber 205, a mixture comprising the solutions and the sample is produced that travels down to the lower chamber mixing or reagent component 212, which lower chamber mixing or reagent component 212 comprises the reagent area 208 with a solid reagent 207. The solid reagent 207 can be dissolved rapidly by the buffer and the resultant solution can be a mixture of sample that may contain analyte(s) of interest, and the assay reagents (e.g., specific binding agents, label detection and capture probes, etc.). For example, a solid reagent 207 can include both detection and capture probes used in the assay that are capable of specifically binding a target analyte. In some embodiments, the SCD can also include a luer lock 211 that locks into a test device for delivery of the reaction mixture for subsequent detection.

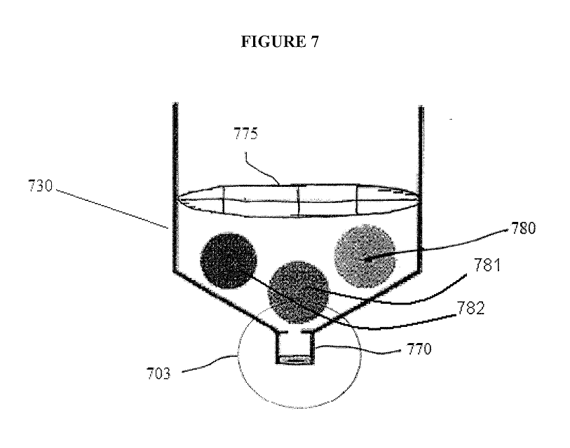

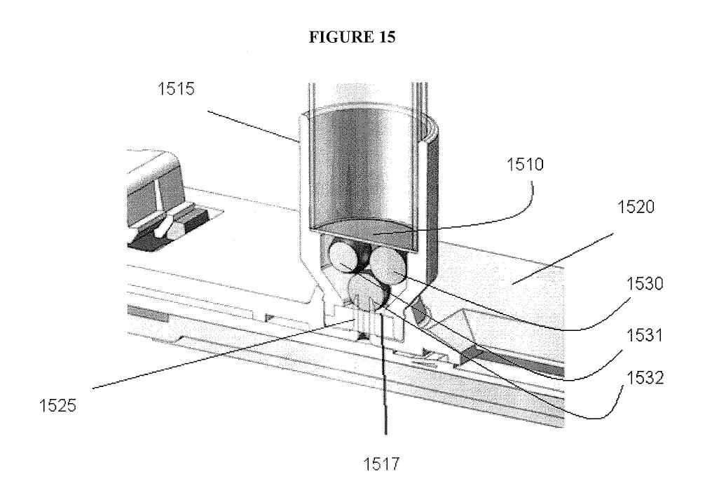

In various embodiments, a SCD comprises the necessary reagents in a solid form (e.g., FIG. 7, 780, 781, 782; FIG. 15, 1530, 1531, 1532). Solid reagent components include, a powder, pill, bead, lyophilized pellet, pressed lyophilized power, dried on solid support (e.g., glass/plastic bead), lyophilized on or in association with a solid support or dried directly in the mixing or lower chamber. Formulating such reagents into solid forms is effected using techniques that are known in the art such as disclosed in CURRENT PROTOCOLS IN IMMUNOLOGY (Coligan, John E. et. al., eds. 1999). In one embodiment, a solid reagent is rehydrated when brought into contact with a liquid sample.

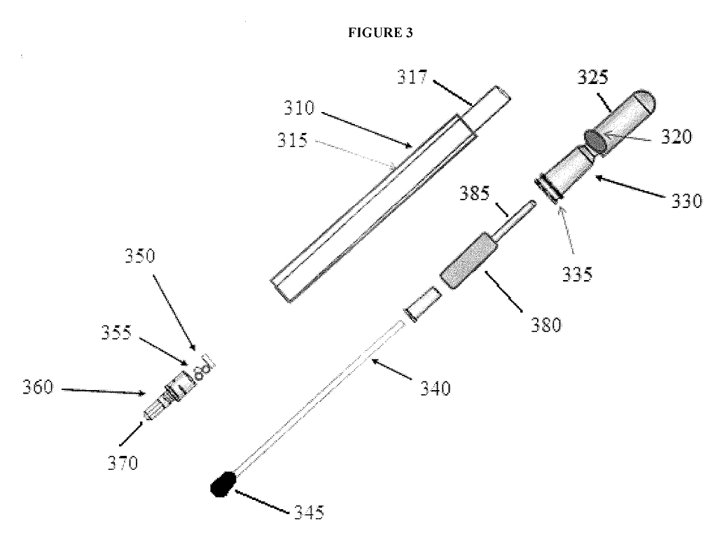

In another embodiment, an SCD is provided as shown in FIG. 3 having an upper chamber 330. In some embodiments, the upper chamber 330 can have at least one breakable seal 320 and a rim 335 that can be in contact with a sample receiving tube 310, for example, through a press-fit. In a further embodiment, when press-fit (also, force-fit) together, an upper chamber and sample receiving tube form an air tight seal and form positive pressure or back pressure that forces uniform release of the contents (e.g., sample mixture) present in the SCD when the SCD is coupled to a test device via the SCD's bottom or distal end FIG. 15. In one embodiment, the bottom or distal end of the SCD releases its content through a split septum that couples to a test device. In a further embodiment, a split septum of the SCD couples to a TD by a cannulae present on the TD.

In a further embodiment, by forming the back pressure, the coupling of a TD and SCD allows for uniform sample flow from the SCD to the TD and through a test membrane, so that capture probe-target analyte-detection probe complexes formed pass through the TD in a uniform time and rate allowing for efficient capture at each addressable line. Uniform flow allows for enhanced assay performance by increasing specificity and/or sensitivity of an assay, which is more critical where targeting multiple different analytes.

In one embodiment, a SCD also can have a sample holder 380 that can be in contact with the upper chamber 330 and a sampling assembly 340. In one embodiment, the sample holder 380 can contain a reagent such as a mucolytic agent (e.g., liquid form or lyophilized). The sample holder can have a tube 385 to facilitate entry into a bulb 325 of the upper chamber 330. For example, the tube 385 can break a valve in the upper chamber 330. The sampling assembly stem 340 can have a sample collection implement 345 to facilitate sample collection. The sampling assembly stem 340 can fit inside a sample receiving tube 310 that can be in contact with a lower chamber mixing or reagent component 360. The lower chamber mixing or reagent component 360 can have an extraction buffer and/or reagent, a mesh membrane 350 and at least one bead 355 that contains a solid reagent (e.g., extraction reagent, immunoassay reagents, such as detection and capture probes, etc.). In some embodiments, the lower chamber mixing or reagent component 360 can have more than one bead 355. For example, the lower chamber can have multiple beads with at least one bead containing a mucolytic agent, one bead containing a capture probe and one bead containing a detection probe. In other embodiments, a single bead can comprise more than one component (e.g., two or more of extraction reagent, detection probe or capture probe). In a further embodiment, a bead can comprise a dye that provides a color indication that the sample is sufficiently mixed with reagent(s) present in the SCD. The formation of color associated with the dye provides an indication of adequate hydration and mixing for reaction of the sample and reagents.

The lower chamber 360 can have a septum 370 that allows a fluid to travel from the lower chamber 360 to a test device. The septum can be made of different materials, including plastic or neoprene, to contain a liquid. The orientation of the SCD is such that the upper chamber component 330 is at the proximal end and the septum 370 is at the distal end.

In some embodiments, the sample receiving tube 310 is made of a soft or flexible material. Materials useful for creation of sample receiving tube 310 are well known in the art, and include soft plastic. In other embodiments, the sample receiving tube 310 can be made of a hard or rigid material. Materials useful for creation of a hard or rigid sample receiving tube 310 are well known in the art, and include, for example, hard plastic or glass. In one embodiment, to allow force-fit of a sample receiving tube to an upper chamber or sample collection implement cap, each component is made of hard plastic or glass to allow force-fit and an air tight seal, which is necessary to provide back pressure. As indicated above, the back pressure allows for uniform flow of a liquid mixture from the SCD to the TD. In a further embodiment, such uniform flow is achieved without any additional force or manipulation, where the SCD is coupled to the TD via the split septum aperture 1090, 1517, when coupled to a cannulae or projection 1420, 1525 from a TD.

In another embodiment, a sample receiving tube 310 is handled during normal operation and a soft or flexible material can be squeezed during use, resulting in potential backflow of liquid away from the sample. This backflow can potentially decrease the amount of fluid reacting with the sample and thereby decrease the accuracy of the analysis. By using a hard or rigid material for the device, an operator can handle the SCD with a decreased backflow of liquid. In another embodiment, the sample receiving tube 310 is composed of more than one tube. For example, the sample receiving tube 310 has a hard or rigid outer tube 315 and a soft or flexible inner tube 317. In another embodiment, the SCD is configured with sleeves which provide a means to move the sides of the tube/casing closer to the swab attached to stem so that as a fluid exits the swab it will stay in close proximity to the swab, so as to improve the efficiency of extracting fluid from the swab. In one embodiment, the sample receiving tube 310 forms a tight fit with an upper chamber component 330, such that an air-tight seal is formed. The air-tight seal can be formed at a rim 335 that forms a seal. In a further embodiment, the rim 335 has a firm seating with the sample receiving tube 310 to create negative air pressure within the SCD upon the sealed closure of the upper chamber component 330 with the sample receiving tube 310.

In another embodiment, the upper chamber 330 forms a tight seal with the sample receiving tube 310, to prevent leakage of air or fluid that could result in incomplete delivery of the upper chamber fluid. In some embodiments, the upper chamber does not contain any vents that could allow air to enter the upper chamber 330 and prevent complete release of fluid. For example, where the valve is a snap-valve and the upper chamber solution is under positive pressure, once the user breaks the snap-valve, the tight seal formed by the upper chamber 330 and the sample receiving tube 310 results in the positive pressure forcing the upper chamber solution from the upper chamber 330 through the sample receiving tube 310, in some instances through the sampling assembly 340 to the lower chamber 360. Thus, in one embodiment, upon coupling of the upper chamber 330 to the sample receiving tube 310, there is no need to create pressure (e.g. pressure created by the user) to move the upper chamber solution from the upper chamber 330 to the lower chamber 360. Thus, by removing the necessity for a user to exert force to move the upper chamber solution to the lower chamber mixing or reagent component 360, this process removes user inconsistencies in exertion of pressure and possible incomplete movement of upper chamber solution to the lower chamber 360, or over-exertion of force that could result in leakage of solution or damage of the device. By having the upper chamber 330 under positive pressure, it also prevents the backflow that can occur upon release of a squeezed bulb.

In some embodiments, the upper chamber 330 can be configured to be removably associated with the sample receiving tube 310. In some embodiments, the upper chamber 330 and sample receiving tube 310 of the sample collection device can be configured such that as the upper chamber 330 is associated with the sample receiving tube 310, pressure is built up within the lumen of the sample receiving tube 310. In some embodiments, the proximal end of the sample receiving tube 310 and the upper chamber 330 are configured so as to be press-fit together, wherein upon assembly a pressurized seal is created that functions to increase the pressure within the bounds of the sample receiving tube 310. The sample receiving tube 310 and upper chamber 330 can form a seal upon mating of the two elements. This seal allows gas, e.g., air pressure, to be built up within the sample receiving tube 310, resulting in a positive pressure compared to the ambient pressure and/or the pressure within the test device. In some embodiments, a gas may be added to the sample receiving tube after the sample receiving tube 310 and upper chamber 330 form a seal, e.g., by introduction of gas via a syringe and needle. In some embodiments, the air pressure trapped within the sample receiving tube is stable, and an air pressure above ambient pressure or the pressure within the test device is maintained for at least 1 minute, or at least 2, 3, 4, 5, 10, 30, 60, 120 or 240 minutes.

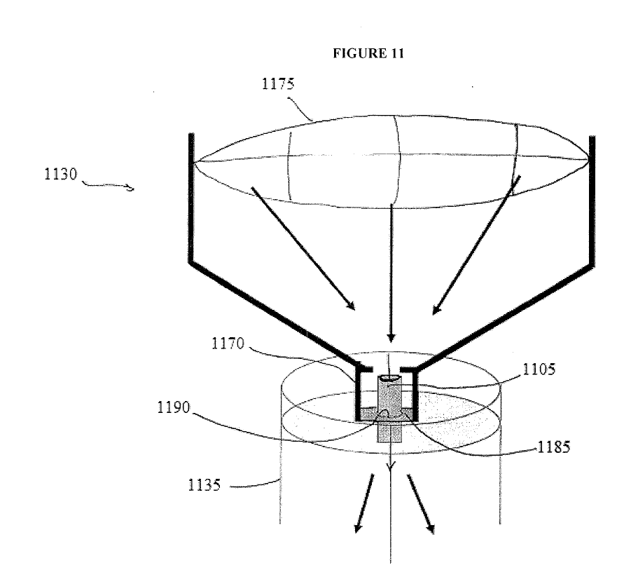

In one embodiment, as shown in FIG. 11, a SCD 1130 is coupled to a second component (e.g., a TD), and a solution comprises sample mixed with reagents and buffers present in the SCD is the forced out of the SCD 1130 and dispensed through the dispensing tip 1170 into a test device 1135 upon mating of the SCD 1130. As noted above, fluid flow from a SCD to a test device can be driven by the built-up pressure within the SCD 1130. For instance, a positive pressure differential may be formed between the SCD 1130 and the test device 1135 due to the trapping of air within the SCD 1130. The pressure differential moves fluid out of the higher pressure SCD 1130 into the lower pressure test device 1135 upon mating of the cannula 1105 and septum 1185, such as through a slit 1190 formed in the septum 1185. The built-up pressure can be stable for a period of time such that mating between the SCD 1130 and the test device 1135 need not occur immediately upon assembly of the SCD 1130. Further, the septum may be configured such that upon removal of the cannula 1105, the septum 1185 reseals and thereby prevents any loss of fluid or dripping of sample. As depicted by the arrows, fluid flows along the pressure gradient from the higher pressure area built up within the SCD 1130 to the relatively lower pressure of the test device 1135 for delivery of sample therein. The SCD includes a membrane 1175 which may hold the reagent pellets in the lower chamber or in some cases may function to hold an archival sample.

In one embodiment, the sampling assembly is not integrated with the housing containing a sample receiving tube. In such a configuration, the sampling assembly is utilized to collect and deliver a sample to a sample receiving chamber. The sample receiving chamber can be open or closed to allow a sample to be introduced into sample receiving tube. It should be understood that any sample receiving tube disclosed herein can be of a variety of geometric shapes, including cylinder, square, triangular or any polygon, as desired. In some embodiments, the housing can comprise one or more sealable apertures that can be opened to add one or more selected reagents, buffers or wash fluids.

For example, in one embodiment, whole blood is drawn into the sample receiving chamber. Subsequently, the sample passes through a membrane (e.g., a membrane to separate blood cells from plasma, allowing the plasma to pass through) into a lower portion of the sample receiving tube to mix with various reagents, for example, necessary for an immunoassay. Immunoreagents necessary to target specific analytes can be pre-selected and disposed as a solid substrate in the SCD or added through an aperture, or is disposed on a membrane.

As the whole blood sample is discharged, the membrane may act as a filter to preclude passage of blood components, thus allowing only plasma to pass through the distal end of the sample receiving tube, which will fit into the Test Device.

In some embodiments, as the solution passes through the sampling implement, an extraction step of a sample occurs (e.g., where solution includes an extraction buffer). Furthermore, the lower chamber can comprise a filter through which an extracted sample flows. For example, if a filter is disposed at the proximal end of the lower chamber, an extracted sample then flows through a filter thereby precluding certain components of the extraction mixture from passing into the reagent area compartment comprising one or more solid reagent beads. Furthermore, a filter means can also function to restrain the reagent bead during SCD transportation and storage and retain the bead(s) in the lower chamber prior to use and hydration. As noted herein, the reagent bead can comprise both the detection and capture probe, or two separate beads can each contain detection or capture probes. In another embodiment, three or more beads can be used, with at least one bead having a mucolytic reagent, one bead having one or more capture probes and one bead having one or more detection probes. In another embodiment, the solution from the upper chamber releases the sample from the sample collection implement (swab) and a lyophilized extraction buffer pellet can be provided in the lower chamber so that extraction can occur in the lower chamber. Alternatively, extraction could occur with the fluid from the upper chamber as the swab is hydrated and also in the lower chamber with lyophilized reagents.

Filtering can allow an analyte of interest to migrate through the device in a controlled fashion with few, if any, interfering substances. Filtering, when present, often provides for a test having a higher probability of success, depending on the type of sample being processed, as would be evident to one of skill in the art (e.g., whole blood sample versus plasma). In another embodiment, the SCD may also incorporate reagents useful to avoid cross-reactivity with non-target analytes that may exist in a sample and/or to condition the sample; depending on the particular embodiment, these reagents may include, but not limited to, non-hCG blockers, anti-RBC reagents, Tris-based buffers, and EDTA. When the use of whole blood is contemplated, anti-RBC reagents are frequently utilized. In yet another embodiment, the SCD may incorporate other reagents such as ancillary specific binding members, fluid sample pretreatment reagents, and signal producing reagents (e.g., substrates necessary for reacting with label conjugates).

In some embodiments, as shown in FIG. 4, the sample receiving tube 450 can contain a separate sampling assembly 457 and hollow shaft 455 for reagent delivery from the upper chamber 410 to the lower chamber 460. The upper chamber 410 can be attached to a sample receiving tube 450. A sample holder 440 can be inside the upper chamber 410 with a tube 430. The upper chamber 410 can have a rim 420 to facilitate an air-tight seal between the upper chamber 410 and sample receiving tube 450. In this embodiment, the sample collection implement, shown here as a swab, is not attached to the upper chamber and can be provided as part of the device before use. Alternatively, any collection device, such as a swab, can be used separate from the SCD to collect a sample and then the sample collection device with a collected sample can be placed inside the SCD for mixing with the fluid from the upper chamber and the reagents of the lower chamber.

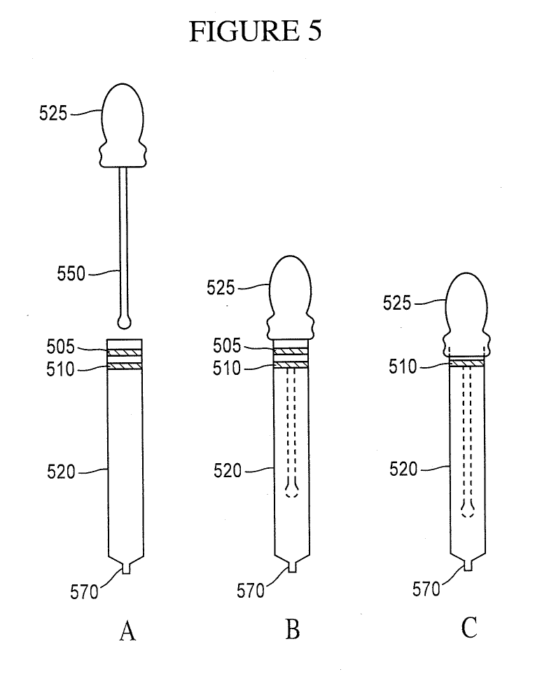

In an embodiment shown in FIGS. 5A-5C, indicator lines 505, 510 may be produced, e.g., printed or otherwise provided, such that they are visible on the outside of the SCD, allowing a user to visualize proper assembly of the upper chamber 525 with the sample receiving tube 520. Such indicator lines can help prevent user error by, for example, preventing air and/or fluid leakage from improperly assembled (i.e., seated) sample collection implements with sample receiving tubes of a SCD. Proper seating and assembly of a SCD is necessary to allow the pressure which may help efficient delivery of the sample into a TD, otherwise a proper air-tight seal may not form or be insufficient. Improper assembly of the SCD may also contribute to non-uniform dispensing of the fluid sample from the SCD into a TD, which may result in poor assay performance. The SCD can have one or more indicator lines. For instance, the SCD may have two indicator lines 505, 510 affixed, engraved, printed or otherwise visible to the outside of the sample receiving tube 520. The upper chamber can, but need not have corresponding indicator lines. In one embodiment, an SCD has two indicator lines (FIG. 5A). When both indicator lines 505, 510 are visible on the upper chamber 525, the user is informed that the SCD upper chamber and sample receiving tube are not assembled properly (FIG. 5B). When only the lower indicator line 510 is visible on the outside of the sample tube 520, the user is informed that the upper chamber 525 has been properly assembled with the sample receiving tube 520 (FIG. 5C). The indicators 505, 510 can be visually distinct such that they are easily read by a user. In one embodiment, the indicators 505, 510 are different colors such that when one color, such as green, is visible the user is informed that the upper chamber 525 is properly seated but when two colors, such as red and green are visible the user is informed that the upper chamber 525 is not properly assembled with the sample receiving tube 520 (FIG. 5B). In FIGS. 5A-5C, provides a non-limiting example of a collection swab 550 attached to the upper chamber 525 and a test device interface 570.

In one embodiment, the lower chamber comprises a small element of absorbent paper, on which a predetermined percentage of the extracted sample is retained for archival purposes. After passing through the collection device and having a portion restrained for archival purposes, the extracted sample contacts a reagent solution or solid (e.g., conjugate bead), and the next assay step takes place as the liquid rapidly dissolves the conjugate bead and allows the reactants to mix with the sample and start the assay.

Test Device (TD)

The present disclosure provides a test device, particularly immunoassay devices, for determining the presence or absence of multiple analytes in a fluid sample. In general, a TD of the present disclosure includes a matrix defining an axial flow path. Typically, the matrix further includes a sample receiving zone, one or more test zones and one or more control zones. In some embodiments, a test region comprises the test and control zones, which are collectively addressable lines.

As used herein in the context of the TD the terms “axial flow membrane”, “lateral flow membrane”, “test membrane”, “test strip” or “matrix” are used interchangeably and refer to features which employ capillary action and/or allows for pressure and/or gravity fluid movement to move or transport the test fluids or employs the movement of fluid separate from capillary action as where fluid is pumped by the accumulation of gas pressure, hydraulic pressure (direct pumping using a piston or rotary, bellows or other type pump on the assay fluids, electrostatic movement due to an electric field, gravity, etc.).

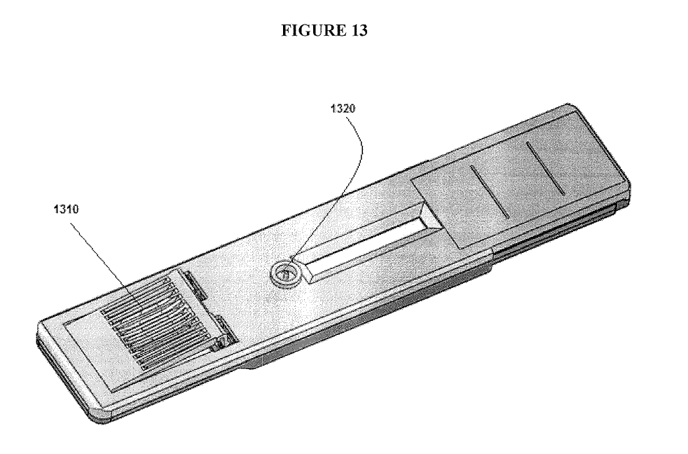

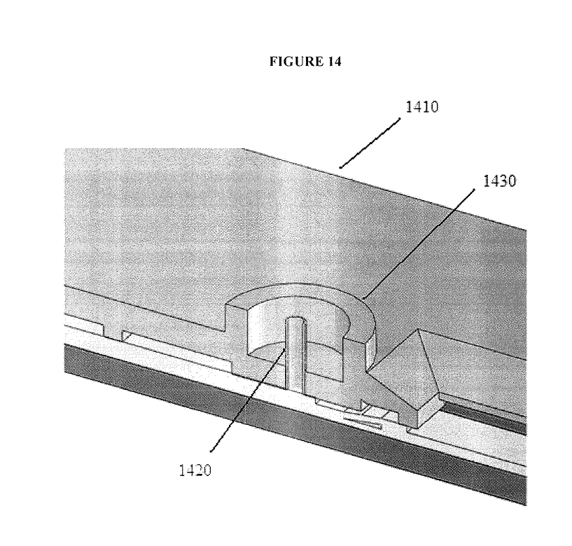

In one aspect of the invention, the Test Device 1410 as depicted in FIGS. 13 and 14 is comprised of an aperture/port 1320,1430 into which the distal end of a SCD of the invention can be engaged, for example, by friction fit, luer lock, adaptor or valve. An aperture/port 1430, provides an opening through which a sample from the SCD flows into the TD. For example, the aperture/port can have a cannula 1420, which in some embodiments will fit into a septum device present in the SCD. A split septum device allows for high flow rates, low priming volume and flexibility to use luer slip or luer lock connections. In some embodiments, a blood separation membrane can be disposed at the port which provides one way flow. In another embodiment, such a membrane can also be disposed in the SCD (e.g., immediately distal to the sample collection implement). In one embodiment, a TD (FIG. 13) comprises a chamber 1310 upstream of the port for coupling to a SCD, wherein the chamber comprises a pouch of wash buffer with a housing or cover.

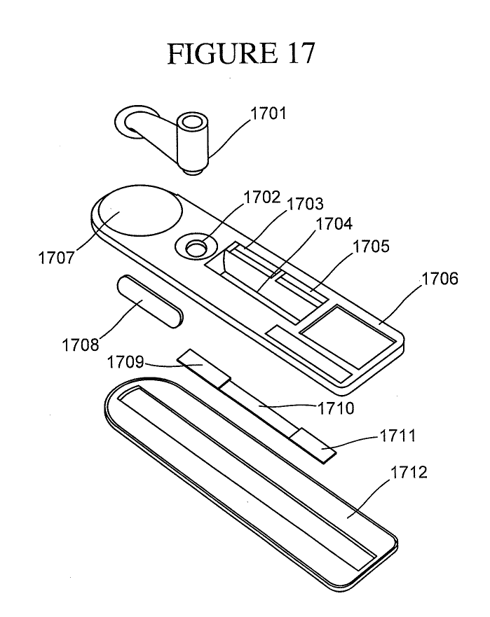

In one embodiment, an illustrative example of a TD is shown in FIG. 17. The TD can have an upper housing 1706 and a lower housing 1712. The TD can have a removable safety cover 1701 disposed over the depressible chamber 1707. The aperture/port 1702 provides an opening through which a sample from the SCD flows into the TD. The TD can have a barcode with information such as patient ID 1703 and lot number 1705.

In one embodiment, the TD comprises two sections, wherein one section comprises a portion where a sample is applied and a second upstream section comprising a wash or running buffer. In another embodiment, the upstream section can comprise one or more compartments which may contain the same or different buffers, wherein each compartment can be separately or simultaneously manipulated to expel its contents.

Upstream of the aperture is a buffer compartment 1708, 1310 that may be in fluid communication with an aperture 1702, 1320 that is upstream of a test membrane comprising a plurality of addressable lines. In one embodiment, a TD aperture 1702, 1320 is in fluid communication with a wicking substrate 1709.

In a further embodiment, a buffer compartment can comprise one or more subcompartments that contain one or more solution(s). Subcompartments in the context of the TD can be made of a pierceable, puncturable, breakable (e.g., ampoule or ampoules) or depressible bladder-like material (e.g., pouch or pouches). As indicated herein, such compartments can be manipulated by applying pressure so as to puncture, break or depress the compartment enough so to release it contents (e.g., user presses chamber cover with finger). In addition, such compartments may be pierced by a lance, stab or appendage that breaks into said compartment upon exertion of force (e.g., thumb pressing down) onto said compartment.

In another embodiment, the buffer compartment itself is semi-rigid, pliable, depressible, or bladder like, thereby providing a means for compacting the compartment to expel any contents therein. Therefore, in some embodiments, a user can exert pressure on the compartment 1708, 1310 that will result in contents therein, whether self-contained or contained in a subcompartment, to be released.

In some embodiments, the compartment 1708, 1310 comprises a solution including but not limited to a wash buffer or chase buffer, which mobilizes or enhances mobilization of the processed sample mixture into the test strip 1710. Generally, such liquid solutions in the compartment can comprise wash buffer, saline or any other desired solution. Furthermore, in some embodiments, such a solution can comprise reagents, enzymes, labels or chemical compounds. The wash buffer can mobilize any unbound label causing it to migrate along the strip past the detection zone thus reducing background. The wash buffer can be optimized to push the assay mixture via hydrostatic pressure and/or to reduce background signal, e.g. europium background. The wash buffer can include about 1%, 2%, 3%, 4%, 5%, 10%, 15%, 20%, 25%, 30%, 35%, 40% or more sucrose. In one embodiment, the wash buffer contains 20% sucrose.

In one embodiment, downstream of the test strip 1710 is disposed an absorbent substrate 1711. In another embodiment, a test membrane can overlap or abut to one or both the wicking substrate and absorptive substrate, respectively. Furthermore, in some embodiments, the TD upper 1706 or lower housing 1712 can comprise identity labels 1703 and 1705, which identify and correspond to an identical identity label on the SCD and can also identify the lot number of the TD (e.g., for quality assurance and tracking purposes). One or more windows 1704, 1610 through the upper housing permits visualization and reading of the results (see also, e.g., FIG. 16).

In another embodiment, the test membrane further comprises an absorbent zone disposed downstream of the last of an addressable line. In one embodiment, a compartment is disposed upstream of the lateral flow 1620 membrane. In another embodiment, a wicking pad is disposed directly below the sample entry aperture.

Suitable materials for manufacturing absorbent substrates include, but are not limited to, hydrophilic polyethylene materials or pads, acrylic fiber, glass fiber, filter paper or pads, desiccated paper, paper pulp, fabric, and the like. For example, the lateral flow membrane absorbent zone may be comprised of a material such as a nonwoven spunlaced acrylic fiber, i.e., New Merge (available from DuPont) or HDK material (available from HDK Industries, Inc.), nonwoven polyethylene treated to improve (e.g., decrease) the hydrophobic property of the material.

Coupling of SCD to TD

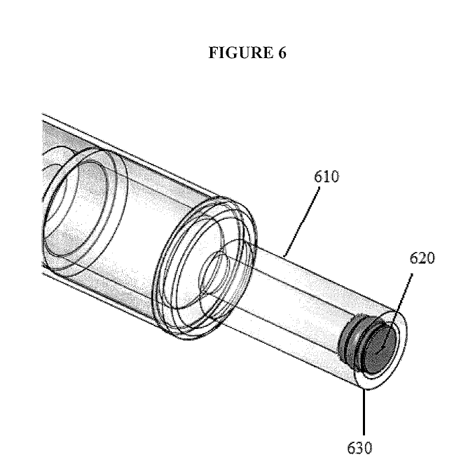

In some embodiments, a SCD comprises a split septum. An illustrative example of an SCD with a narrowed distal end having a split septum is shown in FIG. 6. The SCD 610 has a split septum 620 at the distal end of the SCD 630.

An illustrative example of an SCD distal end in the lower chamber mixing or reagent component 730 is shown in FIG. 7. The distal end of the SCD can contain an outlet region 703 with a reduced-diameter dispensing tip 770. Reagent beads 780, 781, 782, as described above, can be in the lower chamber 730.

In some embodiments, the lower chamber 730 contains a mesh membrane 775 (See also FIGS. 3, 10 and 15, 350, 1075, 1510) that comprises one or more beads 780, 781, 782 within the lower chamber 730.

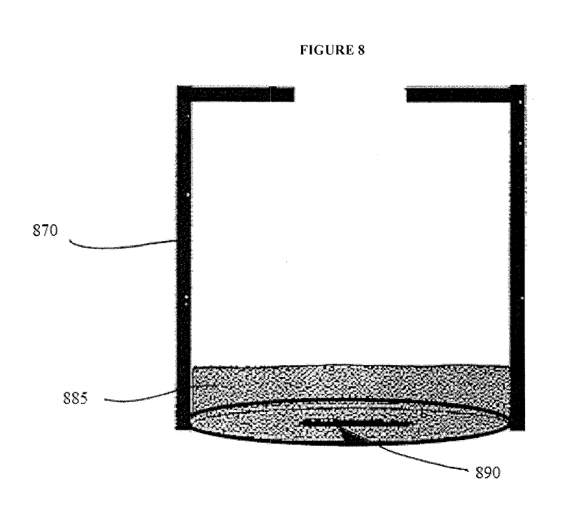

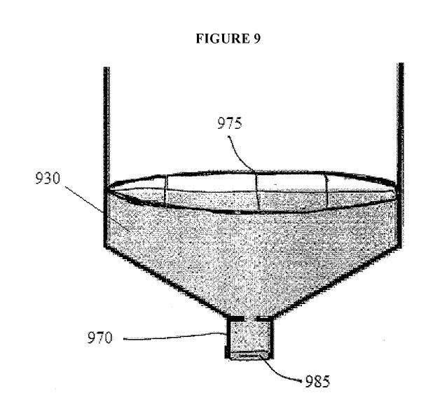

As shown in FIGS. 8 and 9, in one embodiment, the dispensing tip 870, 970 of the lower chamber 930 comprises a septum 885, 985 which may include a slit 890. In a further embodiment, the lower chamber also comprises a mesh membrane 975 that positions and secures the immunoreagents (e.g., bead comprising capture probes and detection probes described herein). In some embodiments, the septum is made of an elastomeric material, such as rubber or neoprene.

In some embodiments, the septum includes a slit. For example, the slit provides a means through which a cannula can be inserted. In some embodiments, the slit retains air trapped within the sample receiving tube and retains the positive pressure created by connecting the sample receiving tube and the upper chamber (also, “sample collection implement”).

In other embodiments, the septum is puncturable, so that when punctured a fluid path is formed between a SCD and TD. In some embodiments, the septum is resealable after puncture. A resealable septum prevents fluid or air from escaping the SCD or any dripping or loss of sample, even after a puncture. In one embodiment, the septum is comprised of an elastomeric material, such as rubber or neoprene, and includes a slit 890. In some embodiments, the septum retains the pressure and fluid within the SCD until it is coupled with a cannulae of a TD to form a fluid channel. The slit allows for firm closure due to the pressure of the rubbery, elastomeric material of the septum 620, 885, 985, but also allows easy insertion and passage of a cannula 1005, 1105, 1235, 1420 through the slit, creating a fluid path to allow fluid flow into the TD.

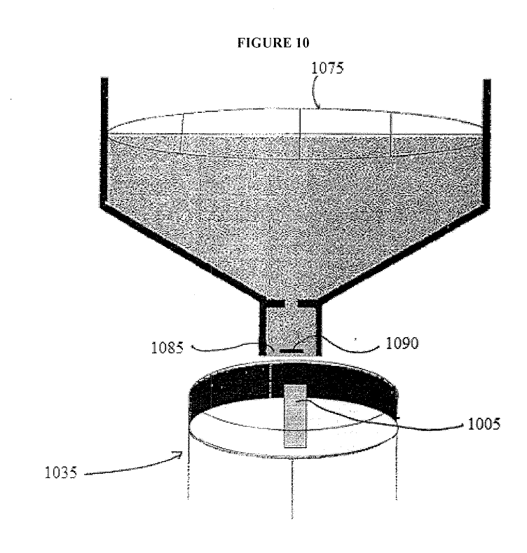

In one embodiment, See FIG. 10, the cannulae 1005, 1105 of the TD 1035 punctures the septum 1085, 1185 of the SCD at a slit 1090. The SCD may include a mesh membrane 1075 to retain the reagent bead(s) in the lower chamber In some embodiments, a cannula 1005, 1105, 1235, 1420 can have any suitable configuration, as known in the art, and may be blunt-tipped or sharpened and may be hollow or solid.

In one embodiment, shown in FIG. 15, the SCD 1515 is depicted as coupled to the test device 1520. As shown, the, the test device includes the lower chamber with reagent beads 1530, 1531 and 1532 held in place with the mesh membrane 1510 and the cannula 1525 of the test device extends though the split septum 1517 of the SCD for smooth delivery of the reaction products of the sample and the specific reagents, detection probe and capture probe, included in the SCD, that react with one or more analytes present in the sample.

Archive Sample. In one embodiment, a means for archiving a portion of a sample is provided. In some embodiments, a SCD or TD, or both, comprise an archival means, which can comprise an absorbent or adsorbent substrate (e.g., paper or membrane), a short capillary tube of defined length, or a small reservoir/compartment for retaining a portion of the sample in the lower chamber.

In some embodiments, an archival filter or membrane is located in a position in the device before the sample encounters the reaction reagents (e.g., 206, 350, 775, 975, 1075, 1175, 1275, 1510).

In another embodiment, an SCD comprises a means for retaining an archive sample. For example, within a SCD lower compartment, filter paper and/or hydrophobic membranes can be configured to retain a sample for archiving purposes. Various combinations of materials are possible for use as the means for archiving, such that one, two, three or more materials may be used alone or in combination. In one embodiment, the means for archiving comprises three disks that may or may not touch each other. The disks can comprise a grid portion and a pad portion, wherein the pad portion is designed to retain an archive sample. The pad portion can be comprised of any absorptive/adsorptive material and can comprise 5, 10, 15, 20, 25, 30, 35, 40, 45 or 50% of the surface area of a disk. Furthermore, the grid portion can comprise three dimensional (“3D”) substrates raised relative to the surface of a disk. Such 3D protrusions can provide a grid into which a reagent bead can be disposed. Such beads can measure in size from about 0.5, 1, 1.5, 2.0, 2.5, 3.0, 3.5, 4, 4.5, 5.0, 5.5, to about 6.0 mm.

In one embodiment, a small compartment that can provide a small reservoir for an archived sample is positioned in the TD adjacent to the port/aperture for delivery of sample to the TD. Such an archive compartment can be configured to be removable or configured such that a substrate onto which the archive sample is disposed, is itself removable from said compartment. For example, a filter/membrane material sized to fit into the compartment will function to collect to a predetermined capacity of sample (e.g., cell, cell components, protein, nucleic acid, etc.). A filter/membrane comprising the archive sample is then removed and appropriately stored, e.g., drying or freezing.

In one embodiment, the archived material is a cell(s) or cellular component, including but not limited to a protein, peptide, protein fragment or nucleic acid molecule. Therefore, samples can be preserved for further testing depending on the type of molecule archived (e.g., protein versus nucleic acid). Furthermore, archive disks provide a means of storing samples and maintain stability of said samples from about 1, 2, 3, 4, 5, 6, 7, 8, 9, 10, 11, 12, 13, 14, 21 to 30 days or longer.

In another embodiment, the archival disks are placed in a preservative solution, which extends storage time for said archive samples from about 1, 2, 3, 4, 5, 6, 7, 8, 9 or 10 weeks. Of course depending on the in-field setting, samples can be stored indefinitely (e.g., once the sample is subjected to freezing).

In another embodiment, a reaction compartment in the lower chamber can be removed from the sample receiving tube and placed in a housing (e.g., plastic tube). In one embodiment, the compartment retains a small volume of sample mixture to which a preservative can be added for storage. In another embodiment, the solutions provided in the upper chamber or a reaction solution in the lower chamber can also include preservatives necessary to archive a liquid sample. Such preservatives are known in the art. See, e.g., U.S. Pat. No. RE29061; Buccholz et al. Transfusion. 1999 September; 39(9):998-1004; Quiagen specialty reagents, available at Quiagen.com. In one embodiment, an archive sample is retained for later testing (e.g., by RT-PCR).

In another embodiment, a SCD does not have any fits or means for retaining an archival sample. An example of an SCD that does not have any frits or means for retaining an archival sample is shown in FIG. 3, wherein a membrane 350 separates the immunoreagents (e.g., 355) from the upper portion of the sample receiving tube 310.

Sample Identification. In one embodiment, an SCD also includes anywhere on the sample collection implement or the sample receiving tube, one or more identifying labels (e.g., barcodes allowing at least 10⁹ unique values) into or onto which information—e.g., patient identification number—can be attached to the sample receiving tube. Identifying labels can also be used to record method, lot, and expiration dating of the TD. The labels can be peel-off and can be self-adhesive. In one embodiment, at least one label is retained on the SCD while peel-off copies can be placed on the TD and/or on any facility paperwork, or an archival reservoir means. An illustrative example of a barcode showing patient ID 1703 and lot number 1705 is shown in FIG. 17. Bar code format will be to a universal standard such as Codabar. In other embodiments, the identifying labels can be signal emitting transponders known in the art, including but not limited to, radio frequency emitter, light emitter or electromagnetic wave emitter.

SCD Compartments. In some aspects of the invention, the SCD comprises one or more compartments in the lower chamber that can include reagents, filters, membranes and reservoirs. In one embodiment the upper chamber of the SCD may comprise one, two or more compartments, each of which can further contain a solution. In some embodiments, such compartments can comprise the same or two different solutions, reagents, buffers, or a combination thereof. Further, multiple compartments can be arranged in series in a lower chamber (e.g., multiple cages in series). In addition, such compartments may be referred to as “subcompartment” or “subcompartments” in the disclosures herein.

In one embodiment, a compartment is distal relative to a sampling implement and contains a liquid or solid reagent component that comprises binding agents that are specific to one or more particular analytes (or analyte type). For example, the liquid or solid reagent component can include a specific binding agent (e.g., antibody) that is capable of specifically binding an analyte that may be present in a sample. In some embodiments, a single reaction or mixing compartment (lower chamber) is utilized in the SCD that is distal to and in fluid communication with the sampling implement. In other embodiments, one or more compartments can be utilized where one compartment functions as a lysis or extraction chamber, while a second compartment distal to the first compartment functions as a reagent-sample mixing chamber. In further embodiments, filtering means may be disposed on the proximal end of one or more compartments, which compartment(s) is disposed distal relative to the sampling implements. Filter means can be utilized to remove certain components from the sample at any point during analysis of the sample, e.g., prior to extraction/lysis, following sample-reagent mixing, during processing or before release from the SCD. Furthermore, the same or different filtering means can be disposed on multiple compartments if such multiple compartments are present in the sample receiving tube.

In order to ensure proper reaction of the reagents and outcome of the analysis, mixing of a sample and binding agents must occur and the sample must come in contact and adequately interact and mix with the binding agents. In one embodiment, the reagent-sample mixing chamber has mixing indicator beads. The beads can be coated with a material that indicates when proper mixing has occurred. For example, the mixing beads may be coated with a red dye, such that during mixing of the sample and binding agents in the presence of the beads, adequate contact and mixing is demonstrated by the solution turning a red color. Generally, the dye should be a releasable, water-soluble dye that is visible upon release to the naked eye. Preferably, the dye does not interact with the sample analyte. A variety of suitable dyes in a variety of colors are known in the art, such as bromoscresol green, bromocresol blue, fuchsin, methyl green, o-cresol red, orange G and safranin O. This dye indicator allows even a novice user to utilize the device and obtain accurate reproducible results by observing the development of the red color as an indication that sufficient mixing of the reagents has occurred. For example, the beads can be designed such that a red color is produced following 5-10 seconds of mixing. The mixing of sample and binding agent may be mixed for 5, 10, 15, 20, 25, 30, 60 or more seconds. Alternatively, the mixing of sample and binding agent may be for 5-10, or 10-15, or 15-20, or 20-30 or 30-60 seconds or greater. Mixing for at least 5 seconds was shown to be sufficient for proper interaction between a sample and binding agents. An example of the mixing is shown in FIG. 22. Mixing for greater periods of time (e.g. 30 seconds) did not significantly improve the reaction results. Mixing can be achieved by several methods, including flicking the SCD, wrist flicking the SCD, and vortexing of the SCD.

Samples. A sample is any material to be tested for the presence and/or concentration of one or more analytes. In general, a biological sample can be any sample taken from a subject, e.g., non-human animal or human and utilized in the TDs. For example, a biological sample can be a sample of any body fluid, cells, or tissue samples from a biopsy. Body fluid samples can include without any limitation blood, urine, sputum, semen, feces, saliva, bile, cerebral fluid, nasal swab, nasopharyngeal swab, nasopharyngeal aspirate, nasal wash, throat swab, urogenital swab, nasal aspirate, spinal fluid, etc. For example, with the use of a nasal swab, a dry polyester swab can be placed into the nostril, along the same line as the roof of your mouth, and left in place for a few seconds. It is then slowly removed with or without a rotating motion. Both nostrils can be tested with the same swab. In some embodiments, a swab used to collect a sample can be part of a sample collection device (SCD). In other embodiments, a swab used to collect a sample can be separate from an SCD, and used to collect a sample prior to placement in an SCD. As another example, with the use of a nasopharyngeal swab, a flexible, thin polyester swab can be placed into the nostril and back to the nasopharynx and left in place for a few seconds. It is then slowly removed with or without a rotating motion. A second swab can be used for the other nostril. As yet another example, with the use of a nasopharyngeal aspirate, nasopharyngeal fluids can be removed by suction, e.g. through a tube. The tube is placed into the nostril along the same line as the roof of the mouth. Suction is applied and the tube is slowly withdrawn with or without a rotating motion. A sample from the other nostril can be collected with the same tube or a different tube in the same way. As yet another example, with the use of a nasal wash, a patient can be seated in a comfortable position with the head slightly tilted back. In some embodiments, the patient can keep the back of their throat closed by saying “K” while the washing fluid (e.g. saline) is placed in the nostril. With a transfer pipette, 1-1.5 ml of fluid can be placed into one nostril at a time. The patient then tilts their head forward and lets the fluid flow into a collection dish. This process can be repeated back and forth alternating nostrils until a total of 10-15 ml of fluid has been used. As yet another example, with the use of a throat swab, a swab is used with pressure to swab both tonsils and back of the throat. The swab is then placed in a provided container. Biological samples can also include any sample derived from a sample taken directly from a subject, e.g., human. For example, a biological sample can be the plasma or serum fraction of a blood sample, protein or nucleic acid extraction of collected cells or tissues, or from a specimen that has been treated in a way to improve the detectability of the specimen, for example, a lysis buffer containing a mucolytic agent that breaks down the mucens in a nasal specimen significantly reducing the viscosity of the specimen and a detergent to lyse the virus thereby releasing antigens and making them available for detection by the assay. A sample can be from any subject animal, including but not limited to, mammals, birds, reptiles, amphibians, fish, and invertebrates. Non-limiting examples of mammals include humans, pigs, horses, cows, mice, cats, dogs or sheep.

Samples can be collected from any biologic or non-biologic source. For example, a sample can be derived from any biological source, such as a physiological fluid, including blood, serum, plasma, saliva or oral fluid, sputum, ocular lens fluid, nasal fluid, nasopharyngeal or nasal pharyngeal swab or aspirate, sweat, urine, milk, ascites fluid, mucous, synovial fluid, peritoneal fluid, transdermal exudates, pharyngeal exudates, bronchoalveolar lavage, tracheal aspirations, cerebrospinal fluid, semen, cervical mucus, vaginal or urethral secretions, amniotic fluid, and the like. Herein, fluid homogenates of cellular tissues such as, for example, hair, skin and nail scrapings and meat extracts are also considered biological fluids. Pretreatment may involve preparing plasma from blood, diluting or treating viscous fluids, and the like. Methods of treatment can involve filtration, distillation, separation, concentration, inactivation of interfering components, and the addition of reagents. Besides physiological fluids, other samples can be used such as water, food products, soil extracts, and the like for the performance of industrial, environmental, or food production assays as well as diagnostic assays. In addition, a solid material suspected of containing the analyte can be used as the test sample once it is modified to form a liquid medium or to release the analyte. The selection and pretreatment of biological, industrial, and environmental samples prior to testing is well known in the art and need not be described further.

Other fields of interest include the diagnosis of veterinary diseases, analysis of meat, poultry, fish for bacterial contamination, inspection of food plants, restaurants, hospitals and other public facilities, analysis of environmental samples including water for beach, ocean, lakes or swimming pool contamination. Analytes detected by these tests include viral and bacterial antigens as well as chemicals including, for example, heavy metals (e.g., lead, mercury, etc.), pesticides, hormones, drugs and their metabolites, hydrocarbons and all kinds of organic or inorganic compounds.

Safety Means. In some embodiments, a safety means 1701 is disposed over the depressible chamber 1707 so that the contents of the chamber cannot be accidentally discharged into the channel in fluid communication with the lateral flow membrane. A safety means can be a cover or flange that is lifted or pulled back to expose the depressible chamber or a push button disposed thereon.

Furthermore, such a safety means can function as an adaptor for a specific cognate adaptor, luer or valve present on the distal end of the SCD. Thus, a safety means can cover an aperture into which the distal end of the SCD is engaged, for example, prior to release of a sample into the TD. In an additional embodiment, a reader is designed so that a TD can only be inserted into a receiving port if the safety cover is first removed. For example, a TD with its safety cover removed indicates that a sample has been introduced into the TD and running buffer has been released from the compartment 1708, 1620 upstream of the aperture (adapter/safety cover). In one embodiment, the aperture is disposed above the wicking pad 1709.

Gap Means. In some embodiments, a TD comprises a gap disposed between the lateral flow membrane (e.g., wicking pad) and the channel in fluid communication with the buffer reservoir. The gap functions to keep any solution contained in the push button reservoir and assay sample separate until the appropriate time according to the assay development. For example, where a user exerts pressure on the compartment upstream 1708, 1620 of the sample aperture, the gap is forced closed and a solution contained in the compartment flows in the direction to and through the wicking pad, thus mobilizing the sample through the test strip. As indicated above, the solution can comprise any desired buffer, reagent, chemical compound, dye, label or bead. It should be understood that the gap embodiments disclosed herein can be adapted to any of the TD configurations disclosed herein. In some embodiments, the gap can be from about 0.5, 1, 1.5, 2, 2.5, 3, 3.5, 4, 5, 6, 7, 8, 9 or 10 mm. In one embodiment the gap is greater than zero and less than 3 mm.

In one embodiment, a SCD-processed sample is introduced into the TD, a chase or running buffer is subsequently released and follows the specimen through the wicking pad and into the test strip, where specifically patterned capture moieties bind their partner capture moieties.

Containers and Solution Release. In one embodiment, the TD is a lateral flow test strip, preferably, though not necessarily, encased in a housing, designed to be read by the reader. In one embodiment, a wash/running buffer solution is comprised in a foil, sac or blister type packet (e.g., similar to ketchup/condiment packet) which is disposed in the TD upstream of the sample entry port. The sac or packet can be designed so that it is symmetric about the two orthogonal axes so that it can be loaded into the TD easily. Therefore, in one embodiment, the cover of the TD disposed over the packet when pressed down can cause the packet to break releasing the contents therein.

In one embodiment, the upstream wash/buffer compartment comprises a soft membrane (e.g., form fill seal pack) or ampoule that is easily ruptured/broken upon exertion of minimal force (e.g., user pressing with finger). Such an onion skin compartment can be further covered by a hard removable cover which prevents accidental breakage of the onion skin. The sample enters the TD through a port and the device may have a narrow channel for recovery of an archival sample.

In another embodiment, the button portion can comprise a piercing appendage that punctures the packet as the button is depressed thus releasing the contents therein. A leaf spring or cantilever spring can rest between the packet and the button and results in pressure exerted on the packet to ensure all the contents are released. Further, the geometry of the TD is configured so that the wash buffer is directed toward the wicking pad. In addition the geometry of the button, spring, and housing also reduces air voids in the packet area allowing the wash buffer to flow in any direction, even against gravity (e.g., uphill), as necessary, but not back into the packet storage area.

The number and size of the holes created, as well as the geometry of the holes created can be adjusted relative to one another in order to allow for predetermined flow of the wash buffer out of the packet. In one embodiment, the piercing appendage (e.g., needle) will provide a fluid resistance barrier on the top of the packet, allowing fluid to exit the lower portion of the packet in the direction of the wicking pad. The piercing needle can also be tapered in order to achieve or enhance this function. In one embodiment, the spring is an integral part of the button, top housing or lower housing or it can be a separate component altogether that is configured to easily fit and seal the wash/running buffer chamber. In one embodiment, the sides of the button are designed to minimize pinch points while the button is depressed. Sides can also be designed to provide a baffle-type function, minimizing the risk of liquid exiting the TD.

In another embodiment, the geometry of the feature that supports the end of the wicking strip is designed to allow the piercing feature (e.g., needle) to pass through the packet and not allow the packet to form a seal between the packet and the support feature. The action of the needle pierces both the wicking pad and the packet. In another embodiment, the piercing is only of the packet with the wicking pad located directly adjacent to the pierced hole.

In one embodiment, the wash/running buffer in the TD is comprised in a breakable/rupturing substrate (e.g., an ampoule). Pressure exerted on a sealing membrane or button breaks the ampoule thus releasing its contents. In one embodiment, a channel, gutter, or trough is designed to direct the buffer to the wicking pad.

In one embodiment, the aperture for receiving the SCD distal end comprises a break-away collar (“Lock Collar”) which attaches to the SCD assembly and breaks away from the TD body as the SCD is removed, thus releasing wash or running buffer from a compartment/reservoir upstream or immediately upstream of said aperture. In yet another embodiment, the Lock Collar when twisted into the lock position allows a sample to be dispensed onto the TD while concurrently releasing buffer or wash buffer from an upstream compartment. For example, the Lock Collar will comprise a geometry of channels, holes or openings that line up with openings, channels or holes of the wash/buffer compartment only when the collar is in the lock position. Such a Lock Collar can be utilized with any of the one or more upstream compartments that can be utilized to deliver a buffer/wash or any other liquid. In an alternative embodiment, the SCD can comprise the Lock Collar which fits into the TD body and twists from an unlock position to a lock position.

Time Delay Means. In any of the embodiments herein directed to a wash/running buffer release from a chamber upstream of the sample (e.g., sample entry port), a time delay feature can be configured into the TD, so that a period of time passes between introduction of the sample and the release of the wash/running buffer. For example, a dry wicking pad substrate swells when wet (i.e., after wash buffer release) and due to the swelling connects to an otherwise disconnected wicking strip. For example, a sample is applied and the ampoule or substrate comprising the wash buffer is broken/ruptured to release the liquid into the dry wicking pad portion, which swells and provides liquid communication to the wicking pad portion containing the sample. The sample/buffer can now run through the test strip via the wicking pad.

In another embodiment, a predetermined length/density of fibrous membrane is placed in between the wash buffer compartment and the wicking membrane, which fibrous membrane can delay the contact of the wash buffer to the wicking membrane thus functioning as a time delay mechanism. Buffer wicks down the fibrous membrane and accumulates on the end of the membrane fibers until it reaches the wicking membrane and flows through with the sample disposed on the wicking membrane. In another embodiment, the buffer accumulates at the ends of the membrane fibers until there is enough volume to bridge a gap separating the fibrous membrane from the wicking membrane.

In other embodiments, a plunger or spring mechanism is configured into the TD, which functions by reducing the compartment/ampoule volume, thus ensuring the contents therein are dispersed onto a wicking pad. A plunger can be moved forward by the user exerting pressure on the button or a spring loaded plunger can be driven forwarded in an automated fashion (e.g., when placed in the reader). The plunger forms a seal as it drives forward so that the liquid's only means of exit is through to the wicking pad.

Test Strips. In one embodiment, the sample is delivered to the test strip by the SCD which includes the stem and swab. Upstream of the test strip is a compartment with wash buffer or other fluid. The test strip includes test zones A, B, and C and control zone. The detection probe, via the conjugate label, will provide a detectable signal. The TD is then inserted into a reader, where the signal from the label is measured and/or detected. In another embodiment, the test strip can be inserted into a moveable tray in the reader after the short assay processing period has completed for a very short read period (˜20 seconds), this allows for a much higher through put of tests with one reader. Further, in another embodiment, the test strip can be inserted into the reader prior to addition of the sample.

In one embodiment, the liquid transport along the test strip is based upon capillary action. In a further embodiment, the liquid transport along the matrix is based on non-bibulous lateral flow, wherein all of the dissolved or dispersed components of the liquid sample are carried at substantially equal rates and with relatively unimpaired flow laterally through the matrix, as opposed to preferential retention of one or more components as would occur, e.g., in materials that interact, chemically, physically, ionically or otherwise with one or more components. See for example, U.S. Pat. No. 4,943,522, hereby incorporated by reference in its entirety.

Any suitable material can be used to make the devices disclosed herein, such material including a rigid or semi-rigid, non-water-permeable material, such as glass, ceramics, metals, plastics, polymers, or copolymers, or any combination thereof. In some embodiments, either the SCD or TD comprise a plastic, polymer or copolymer such as those that are resistant to breakage, such as polypropylene, polyallomer, polycarbonate or cycloolefins or cycloolefin copolymers. Furthermore, devices of the invention can be made by appropriate manufacturing methods, such as, but not limited to, injection molding, blow molding, machining or press molding.

As used herein, test strip substrate refers to the material to which a partner capture moiety is linked using conventional methods in the art. A variety of materials can be used as the substrate, including any material that can act as a support for attachment of the molecules of interest. Such materials are known to those of skill in this art and include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone (PVP), rayon, nylon, polyethylene, polypropylene, polybutlyene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamide, polyacrylamide blends, co-polymers of vinyl and acrylamide, methacrylates, methacrylate derivatives and co-polymers, other polymers and co-polymers with various functional groups, latex, butyl rubber and other synthetic rubbers, silicon, glass, paper, natural sponges, insoluble protein, surfactants, red blood cells, metals, metalloids, magnetic materials, or other commercially available media or a complex material composed of a solid or semi-solid substrate coated with materials that improve the hydrophilic property of the strip substrate, for example, polystyrene, Mylar, polyethylene, polycarbonate, polypropylene, polybutlyene, metals such as aluminum, copper, tin or mixtures of metals coated with dextran, detergents, salts, PVP and/or treated with electrostatic or plasma discharge to add charge to the surface thus imparting a hydrophilic property to the surface.

In one embodiment, the lateral flow membrane is comprised of a porous material such as high density polyethylene sheet material manufactured by Porex Technologies Corp. of Fairburn, Ga., USA. The sheet material has an open pore structure with a typical density, at 40% void volume, of 0.57 gm/cc and an average pore diameter of 1 to 250 micrometers, the average generally being from 3 to 100 micrometers. In another embodiment, the label zone is comprised of a porous material such as a nonwoven spunlaced acrylic fiber (similar to the sample receiving zone), e.g., New Merge or HDK material. Often, the porous material may be backed by, or laminated upon, a generally water impervious layer, e.g., Mylar. When employed, the backing is generally fastened to the matrix by an adhesive (e.g., 3M 444 double-sided adhesive tape). Typically, a water impervious backing is used for membranes of low thickness. A wide variety of polymers may be used provided that they do not bind nonspecifically to the assay components and do not interfere with flow of the fluid sample. Illustrative polymers include polyethylene, polypropylene, polystyrene and the like. On occasion, the matrix may be self-supporting. Other membranes amenable to non-bibulous flow, such as polyvinyl chloride, polyvinyl acetate, copolymers of vinyl acetate and vinyl chloride, polyamide, polycarbonate, polystyrene, and the like, can also be used. In yet another embodiment, the lateral flow membrane is comprised of a material such as untreated paper, cellulose blends, nitrocellulose, polyester, an acrylonitrile copolymer, and the like. The label zone may be constructed to provide either bibulous or non-bibulous flow, frequently the flow type is similar or identical to that provided in at least a portion of the sample receiving zone. In a frequent embodiment, the label zone is comprised of a nonwoven fabric such as Rayon or glass fiber. Other label zone materials suitable for use include those chromatographic materials disclosed in U.S. Pat. No. 5,075,078, which is herein incorporated by reference.

In another embodiment, the test strip substrate is treated with a solution that includes material-blocking and label-stabilizing agents. Blocking agents include bovine serum albumin (BSA), methylated BSA, casein, acid or base hydrolyzed casein, nonfat dry milk, fish gelatin, or similar. Stabilizing agents are readily available and well known in the art, and may be used, for example, to stabilize labeled reagents. In some embodiments, the upstream compartment containing a solution can comprise multiple ampoules, which can be selectively punctured or broken to release their contents. Therefore, in one embodiment, blocking reagents are contained in one ampoule which is utilized to pre-treat (e.g., “block”) the test strip (i.e., lateral flow membrane), while the additional ampoule is reserved for washing the sample through the test strip.

Zones, Labels and Reagents. In various disclosures herein, the test strip/lateral flow membrane comprises multiple test zones. Test zones generally contain a pre-selected partner capture moiety, where a pre-selected region comprises capture moieties that are partners for capture moieties conjugated to analyte-specific binding agents, such as monoclonal antibodies. In some embodiments, the capture probes may include multiple types of labels to detect one or more analytes and also for the control. These multiple types of labels reagent can be detected using various readers, such as a reader capable of detecting different wavelengths from fluorescent labels, or may be detected visually or with a reader able to detect different wavelengths or colors. Alternatively, the same label may be utilized for each analyte. Thus, one labeled reagent can be differentiated from another labeled reagent if utilized and captured in the same device by differentiating the label detected and/or the analyte can be determined by knowing which addressable line provided a result. Frequently, the ability to differentially detect the labeled reagents having different specificities based on the label component alone is not necessarily due to the presence of defined test and control zones in the device, which allow for the accumulation of labeled reagent in designated zones.

In some embodiments, each Analyte Binding Set includes detection probes in which the specific binding agent is conjugated to a different fluorescent label emitting a different wavelength. Therefore, where a plurality of Analyte Binding Sets are provided in a SCD, each Analyte Binding Set utilizes a label different than any other Analyte Binding Set. For example, a first group of antibodies which specifically bind to influenza A can be conjugated to one type of fluorescent label (i.e., detection probe specific binding agents conjugated to a first fluorescent label), while second and subsequent groups of specific binding antibodies (i.e., detection probe specific binding agents conjugated to a second and subsequent fluorescent labels) for example, to influenza B can each comprise distinguishable detection binding agents conjugated to different fluorescent labels. Of course, it should be evident that detection probes can also utilize the same label or the Analyte Binding Sets may use various different labels, such as, fluorescent label(s), metal(s), chromophore(s), or any other appropriate label. In one embodiment, the fluorescent labels emit wavelengths that are sufficiently distinct so that several test lines can be differentiated.

The present description provides for the development and use of single or multiple control zones in a single immunoassay device that are positioned in a predetermined manner relative to individual test zones thereby allowing easy identification of each of the one or more analytes of interest tested for in the device. The present description further provides for the making of control zones of various shapes, physical or chemical identities, and colors. In part, the use of such control zones allows for immunoassay devices that are easy to use, and allow for the identification of multiple analytes during a single assay procedure.

In one embodiment, the TD does not include any reagents contained therein that are capable of specifically binding to an analyte (e.g., antibody that is specific for H5N1 or H1N1). In such embodiments, reagents which bind to the analyte(s) of interest typically will be present in an SCD. The TD may include a capture moiety partner capable of specifically binding to the cognate capture moiety partner of the capture probe and thus capturing the analyte on the test zone addressable line.

The test region generally includes one or more control zone that is useful to verify that the sample flow is as expected. Each of the control zones typically comprise a spatially distinct region that often includes an immobilized member of a specific binding pair which reacts with a labeled control reagent. In some embodiments, the control zone contains an authentic sample of the analyte of interest, or a fragment thereof. In such embodiments, one type of labeled reagent can be utilized (e.g., the labeled reagent will bind both to the analyte and the control), wherein the fluid sample containing the labeled reagent flows to the test and control zones. Labeled reagent not bound to an analyte of interest will then bind to the authentic sample of the analyte of interest positioned in the control zone. In such embodiments, typically the assay will be configured in such a way as to comprise excess labeled reagent (e.g., sufficient to bind both analyte and control). In another embodiment, the control zone contains antibody that is specific for, or otherwise provides for the immobilization of, the labeled reagent. In operation, a labeled reagent is restrained in each of the one or more control zones, even when any or all the analytes of interest are absent from the test sample.

In some embodiments, a labeled control reagent is introduced into the fluid sample flow either in the SCD or in the TD. For example, in the TD, control reagents can be included in the upstream solution/buffer reservoir, which are described herein. In another example, the labeled control reagent may be added to the fluid sample before the sample is applied to the TD, e.g., present in the mixing subchamber in the SCD.

Exemplary functions of the labeled control reagents and zones include, for example, the confirmation that the liquid flow of the sample effectively solubilized and mobilized the labeled reagents from the SCD, which are captured in one or more defined test zones. Furthermore, controls can confirm that a sufficient amount of liquid traveled correctly through the test strip test and control zones, such that a sufficient amount of partner capture moieties could react with the corresponding specific capture moiety complexed to a specific analyte (i.e., via the antigen specific binding agent). Further, control reagents confirm that the immunocomplexes (e.g., analyte-analyte specific binding agent) migrate onto the test region comprising the test and control zones, cross the test zone(s) in an amount such that the accumulation of the labeled analyte would produce a visible or otherwise readable signal in the case of a positive test result in the test zone(s). Moreover, an additional function of the control zones may be to act as reference zones which allow the user to identify the test results which are displayed as readable zones.

Since the TD can incorporate one or more control zones, the labeled control reagent and their corresponding control zones are preferably developed such that each control zone will become visible with a desired intensity for all control zones after fluid sample is contacted with the device, regardless of the presence or absence of one or more analytes of interest.

In one embodiment, a single labeled control reagent will be captured by each control zone on the test strip. Frequently, such a labeled control reagent will be deposited onto or in the zone in an amount exceeding the capacity of the total binding capacity of the combined control zones if multiple control zones are present. Accordingly, the amount of capture reagent specific for the control label can be deposited in an amount that allows for the generation of desired signal intensity in the one or more control zones, and allows each of the control zones to restrain a desired amount of labeled control-reagent. At the completion of an assay, each of the control zones preferably provides a desired and/or pre-designed signal (in intensity and form). Examples of contemplated pre-designed signals include signals of equal intensities in each control zone, or following a desired pattern of increasing, decreasing or other signal intensity in the control zones.

In another embodiment, each control zone will be specific for a unique control reagent. In this embodiment, the label zone may include multiple and different labeled control reagents, equaling the number of control zones in the assay, or a related variation. Typically, each of the labeled control reagents can become restrained in one or more pre-determined and specific control zone(s). These labeled control reagents can provide the same detectible signal (e.g., be of the same color) or provide distinguishable detectible signals (e.g., have different colored labels or other detection systems) upon accumulation in the control zone(s).

In yet another embodiment, the control zones may include a combination of two types of control zones described in the previous embodiments. For example, one or more control zones are able to restrain or bind a single type of labeled control reagent, and other control zones on the same test strip will be capable of binding one or several other specifically labeled control reagents.

In one embodiment, the labeled control reagent comprises a detectible moiety coupled to a member of a specific binding pair. Typically, a labeled control reagent is chosen to be different from the reagent that is recognized by the means which are capable of restraining an analyte of interest in the test zone. Further, the labeled control reagent is generally not specific for the analyte. In a frequent embodiment, the labeled control reagent is capable of binding the corresponding member of a specific binding pair or control capture partner that is immobilized on or in the control zone. Thus the labeled control reagent is directly restrained in the control zone.

In another embodiment, the detectable moiety which forms the label component of the labeled control reagent is the same detectible moiety as that which is utilized as the label component of the analyte of interest labeled test reagent. In a frequent embodiment, the label component of the labeled control reagent is different from the label component of the labeled test reagent, so that results of the assay are easily determined. In another frequent embodiment, the control label and the test label include colored beads, e.g., colored latex. Also frequently, the control and test latex beads comprise different colors.

In a further embodiment, the labeled control reagent includes streptavidin, avidin or biotin and the control capture partner includes the corresponding member of such specific binding pairs, which readily and specifically bind with one another. In one example, the labeled control reagent includes biotin, and the control capture partner includes streptavidin. The artisan will appreciate that other members of specific binding pairs can alternatively be used, including, for example, antigen/antibody reactions unrelated to analyte. In yet other embodiment, capture partners can include any of the binding moieties disclosed herein.

The use of a control zone is helpful in that appearance of a signal in the control zone indicates the time at which the test result can be read, even for a negative result. Thus, when the expected signal appears in the control line, the presence or absence of a signal in a test zone can be noted.

In still further embodiments, a control zone comprising a mark that becomes visible in the test region when the test region is in a moist state is utilized. Control zones of this type are described in U.S. patent application Ser. No. 09/950,366, filed, Sep. 10, 2001, currently pending and published as U.S. patent application Publication No. 20030049167, and Ser. No. 10/241,822, filed Sep. 10, 2002, currently pending and published as U.S. patent application Publication No. 20030157699.

In some embodiments, one or more control zones of this type are utilized. In another embodiment, a combination of control zones of the type utilizing labeled control reagents and control zone and of the type that display the control zone when in a moist state can be used. This allows for control zones while also allowing use of a reagent-based control zone to ascertain that the re-solubilization and mobilization of the reagents in SCD-processed samples has been effective. Such embodiments also allow for determination that the specific reactions took place as expected along the path defined by, for example, the TD, wick, test strip and absorbent pad. The present disclosure also includes the use of one or more control zones that become visible when the test region is in the moist state for each of the control zones of an assay, except the control zone on the distal or downstream end of the test strip.

Multi-analyte Assays. The present description further provides means to build a rapid, multi-analyte assay, which is needed in many fields of environmental monitoring, medicine, particularly in the field of infectious disease. For example, contemplated devices include those useful for the differential diagnosis of Flu A or Flu B, and subtypes thereof (e.g., Flu A, H5N1 or H1N1) which may result in different treatments, or the differential diagnosis of Flu A, Flu B, and/or RSV in one step. Such devices permit the use of a single sample for assaying multiple analytes at once, and beneficially allows for a considerable reduction of the hands-on time and duration of the diagnostic process for the benefit of the doctor, or user in general. As such, a plurality of immunoreagents can be utilized in an SCD of the invention, where said plurality comprises populations of specific probes, comprising specific binding agents conjugated respectively to label and capture moieties. Typically, a plurality of immunoreagents comprise multiple populations, each specific for a different analyte as compared to other populations within the plurality. For example, the plurality of immunoreagents can be specific for several types of one pathogen (e.g., Flu A, H5N1 and H1N1) or several different pathogens (e.g., Flu A, Flu B, and RSV).

A variety of analytes may be assayed utilizing devices and methods of the present disclosure. In a particular device useful for assaying for one or more analytes of interest in a sample, the collection of analytes of interest may be referred to as a panel. For example, a panel may comprise any combination of influenza A, influenza B, influenza A subtypes, respiratory syncytial virus (RSV), adenovirus, and/or different types of Parainfluenza viruses (for example Types 1, 2, 3 etc.). Another panel may comprise a selection of one or more of upper respiratory infection including, for example, Streptococcus pneumoniae, Mycoplasma pneumoniae and/or Chlamydia pneumoniae. Yet another panel can be devised for the diagnosis of sexually transmitted diseases including, for example, diseases caused by Chlamydia, Trichomonas and/or Gonorrhea. In each case, a particular panel is readily obtained by incorporating a different set of detection and capture probes in the SCD devised to provide signals on the TD for a particular series of analytes, which is described herein. Therefore, a particular SCD will provide all the reagents necessary to detect a particular panel of analytes. In some embodiments, analytes are detected using a TD employing test strips that have detection reagents that are not specific for the analytes of interest, but contain binding partners specific for an analyte-binding reagent supplied from the SCD. Thus, a single TD can be used with SCDs comprising immunoreagents for a different panel of analytes, providing enhanced efficiency and cost effectiveness. In other embodiments, a broad scope TD can comprise non-specific capture probes for several series of analytes from related or distinct pathogens, e.g., detection of HIV and HCV antigens; HIV and tuberculosis, Influenza A, B, and subtypes of A, bacterial and viral infections.

For example, a panel may optionally include a variety of analytes of interest, including SARS-associated coronavirus, influenza A; a hepatitis panel comprising a selection of hepatitis B surface Ag or Ab, hepatitis B core Ab, hepatitis A virus Ab, and hepatitis C virus; a phospholipids panel comprising a selection of Anticardiolipin Abs (IgG, IgA, and IgM Isotypes); an arthritis panel comprising a selection of rheumatoid factor, antinuclear antibodies, and Uric Acid; an Epstein Barr panel comprising a selection of Epstein Barr Nuclear Ag, Epstein Barr Viral Capsid Ag, and Epstein Barr Virus, Early Antigen; other panels include HIV panels, Lupus panels, H. Pylori panels, toxoplasma panels, herpes panels, Borrelia panels, rubella panels, cytomegalovirus panels, panels testing for recent myocardial infarction with analytes comprising an isotype of Troponin with myoglobin and/or CKMB and many others. One of skill in the art would understand that a variety of panels may be assayed via the immunoassays utilizing the devices disclosed herein. Immunoassay methods are known in the art. See, e.g., CURRENT PROTOCOLS IN IMMUNOLOGY (Coligan, John E. et. al., eds. 1999).

Numerous analytical devices known to those of skill in the art may be adapted to detect multiple analytes. By way of example, dipstick, lateral flow and flow-through devices, particularly those that are immunoassays, may be modified in accordance herewith in order to detect and distinguish multiple analytes. Exemplary lateral flow devices include those described in U.S. Pat. Nos. 4,818,677, 4,943,522, 5,096,837 (RE 35,306), 5,096,837, 5,118,428, 5,118,630, 5,221,616, 5,223,220, 5,225,328, 5,415,994, 5,434,057, 5,521,102, 5,536,646, 5,541,069, 5,686,315, 5,763,262, 5,766,961, 5,770,460, 5,773,234, 5,786,220, 5,804,452, 5,814,455, 5,939,331, 6,306,642. Other lateral flow devices that may be modified for use in distinguishable detection of multiple analytes in a fluid sample include U.S. Pat. Nos. 4,703,017, 6,187,598, 6,352,862, 6,485,982, 6,534,320 and 6,767,714. Exemplary dipstick devices include those described in U.S. Pat. Nos. 4,235,601, 5,559,041, 5,712,172 and 6,790,611. It will be appreciated by those of skill in the art that the aforementioned patents may and frequently do disclose more than one assay configuration and are likewise referred to herein for such additional disclosures. Advantageously, the improvements described herein are applicable to various assay, especially immunoassay, configurations.

SCDs or TDs of the invention can be configured to be utilized with existing analyte detection systems. For example, an SCD of the invention can be configured for use with an existing TD, or an existing TD can be configured/modified pursuant to disclosures herein for a TD. Some exemplary devices that can be modified in such a fashion include dipstick, lateral flow, cartridge, multiplexed, microtiter plate, microfluidic, plate or arrays or high throughput platforms, such as those disclosed in U.S. Pat. Nos. 4,235,601, 4,632,901, 5,559,041, 5,712,172, and 6,790,611 6,448,001, 4,943,522, 6,485,982, 6,656,744, 6,811,971, 5,073,484, 5,716,778, 5,798,273, 6,565,808, 5,078,968, 5,415,994, 6,235,539, 6,267,722, 6,297,060, 7,098,040, 6,375,896, 4,818,677, 4,943,522, 5,096,837 (RE 35,306), 5,096,837, 5,118,428, 5,118,630, 5,221,616, 5,223,220, 5,225,328, 5,415,994, 5,434,057, 5,521,102, 5,536,646, 5,541,069, 5,686,315, 5,763,262, 5,766,961, 5,770,460, 5,773,234, 5,786,220, 5,804,452, 5,814,455, 5,939,331, and 6,306,642. Other lateral flow devices that may be modified for use in distinguishable detection of multiple analytes in a fluid sample include U.S. Pat. Nos. 4,703,017, 6,187,598, 6,352,862, 6,485,982, 6,534,320 and 6,767,714, 7,083,912, 5,225,322, 6,780,582, 5,763,262, 6,306,642, 7,109,042, 5,952,173, and 5,914,241. Exemplary microfluidic devices include those disclosed in U.S. Pat. Nos. 5,707,799, 5,837,115 and WO2004/029221. Each of the preceding patent disclosures is incorporated by reference herein in its entirety.

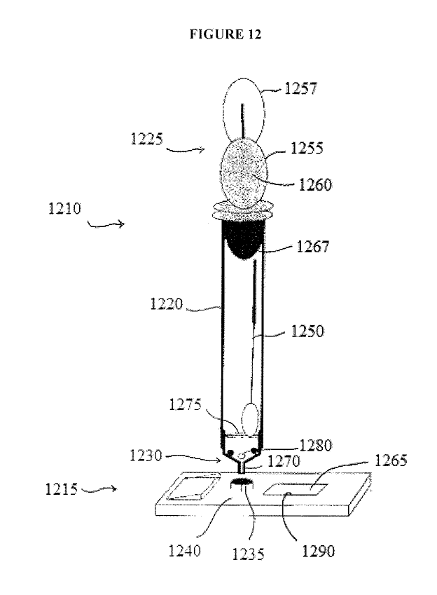

In one embodiment, see FIG. 12 a user collects a specimen using a sample collection implement (e.g., swab) on a sampling assembly 1250 and then inserts it into a sample receiving tube 1220. The upper chamber 1225 is then press-fit into the open, proximal end of the sample receiving tube 1220. The user confirms proper seating of the upper chamber 1225 into the sample receiving tube 1220 by visually inspecting the presence of one or more indicators 505, 510 on the outside of the sample collection tube. In one embodiment, if only indicator 510 is visible from the outside of the sample receiving tube 1220, the upper chamber 1225 is seated properly and a pressurized seal is formed. Once the upper chamber 1225 of the sample collection device 1210 is press-fit onto the proximal open end of the sample receiving tube 1220 forming a pressurized, sealed unit, the valve 1267 in the extraction reagent chamber 1255 containing an extraction reagent 1260 is opened, for example by squeezing or snapping, and the extraction reagent 1260 moves out of the upper chamber 1225 and into the sample tube 1220. The upper chamber may include another compartment or bulb 1257 which may be manipulated manually to release the contents from any of the compartment of the upper chamber. In one embodiment, the extraction reagent in the SCD is contained in a breakable/rupturing substrate (e.g., an ampoule). Pressure exerted on a sealing membrane or button breaks the ampoule thus releasing its contents. The extraction reagent 1260 reconstitutes the lyophilized reagent beads 1280 contained in the lower chamber 1230 and retained by a mesh membrane 1275, wets the sampling implement 1250, and extracts the sample from said implement, in some cases, aided by the user for example by rapid shaking or other agitation of the SCD 1210. The reconstituted reagent beads 1280 and extracted sample react such that analytes of interest within the extracted sample bind with capture probes forming immunocomplexes ready for detection with the TD.

The extracted sample containing the immunocomplexes is then dispensed from the SCD 1210 into a TD 1215, e.g., by using the pressure trapped or built-up during assembly of the SCD 1210 or gravity flow. The dispensing tip 1270 of the SCD 1210 is inserted into the port 1235 of the TD 1215 such that the cannula 1005 inserts through the slit 890 of the septum 885 spanning the dispensing tip 1270 of the sample receiving tube 1220 creating a flow path. The built-up pressure and/or gravity forces the fluid sample through the flow path into the TD 1215. The port 1235 is in fluid communication with a test strip 1265 such as a lateral flow membrane in the TD 1215. The test zones of the test strip 1265 are visible through and opening or window 1290 provided in the upper surface of the housing 1240 of the test device. Upon removal of the cannula 1005 from the septum 1085 the slit 1090 reseals and prevents any spillage, aerosol or contamination. The immunocomplexes within the fluid sample bind or hybridize in predetermined lines or spots on the lateral flow membrane 1265. Detection probes (via conjugate labels contained thereon) provide a detectable signal which can subsequently be read (such as with a scanning device or reader) to determine which analytes are present in the sample (e.g., by detecting the presence of a detectable signal at one or more defined lines on the test device).

Readers.

The systems and methods described herein can include an immunoassay device in combination with a reader, particularly a reader with a built-in computer, such as a reflectance and/or fluorescence based reader. Such readers may also contain data processing software employing data reduction and curve fitting algorithms, optionally in combination with a trained neural network for accurately determining the presence and/or concentration of analyte in a biological sample. As used herein, a reader refers to an instrument for detecting and/or quantization data, such as on test strips comprised in a TD. The data may be visible to the naked eye, but does not need to be visible (e.g., radioactive, non-visible flourescence emitters). The methods can include the steps of performing an immunoassay on a patient sample, reading the data using a reflectance and/or fluorescence based reader and processing the resultant data using data processing software employing data reduction. Preferred software includes curve fitting algorithms, optionally in combination with a trained neural network, to determine the presence or amount of analyte in a given sample. The data obtained from the reader then can be further processed by the medical diagnosis system to provide a risk assessment or diagnosis of a medical condition as output. In alternative embodiments, the output can be used as input into a subsequent decision support system, such as a neural network, that is trained to evaluate such data.

In various embodiments, the reader can be a reflectance, transmission, fluorescence, chemo-bioluminescence, magnetic or amperometry reader (or two or more combinations), depending on the signal that is to be detected from the TD. (e.g., LRE Medical, USA). In one embodiment, the reader comprises a receiving port designed to receive a TD, but where the TD can only be inserted into the receiving port if a depressible (e.g., push button) means upstream of the sample entry aperture has been depressed allowing the TD to fit into the receiving port. Thus, in such an embodiment, the TD is placed in a reader only when the contents of the solution reservoir (e.g., wash buffer) has been released, ensuring that the sample has been “run-through” the lateral flow membrane comprised in the TD.

In one embodiment, the reader is a UV LED reader which detects a fluorescence signal. The fluorescence signal is excited by a light emitting diode that emits in the UV region of the optics spectrum and within the absorbance peak of the fluorescence signal (e.g., lanthanide label). The emitted fluorescence signal is detected by a photodiode and the wavelength of the signal detected may be limited using a long pass filter which blocks stray emitted light and accepts light with wavelengths at and around the peak emission wavelength of the fluorescence emitting label. In other embodiments, the long pass filter may be replaced by a band pass filter. Furthermore, the excitation light may be limited by a band pass filter. In another embodiment, the diode is a UV laser diode. Any conventional UV, LED or photodiode may be utilized.

In any such embodiments, the excitation source and the detector can be mounted in a single machine or molded block. For simplified reading of the fluorescent signals generated on the test strip. In a further embodiment, such a machine also comprises hard standards.

In one embodiment, the axis of the excitation light is at 90 degrees to the TD or test strip comprised in a TD. Further, the axis of the emitted light is at an angle other than 90 degrees to the test strip.

In one embodiment the wavelength of the excitation light is limited by a short pass filter. In yet another embodiment the wavelength of the excitation light is limited by a combination of band pass filter and short pass filter. In yet a further embodiment, the wavelength of the detected light is limited by a combination of band pass and long pass filter. The reader can be configured to detect any of the signal emitters/labels described herein. In one embodiment, the label is any of the lanthanides described herein. In a further embodiment, the lanthanide used is Europium.

As indicated herein, in one embodiment, the reader is configured to comprise one or more hard standards. Thus, the reader can be machined to provide a implement (e.g., a jig) to hold 0.5, 0.75, 1, 1.25, 1.5, 1.75, 2, 2.25, 2.5 or 3 mm standards (e.g., encased in acrylic as described herein), which standard is disposed on about 3, 4, 5, or 6 mm centers. (e.g., See FIG. 5).

In one embodiment, the reader is adapted with a receiving port for the TD, which itself can be configured with a safeguard. In one embodiment, the reader will accept, but not process, the TD if the push button has not been depressed, or the reader will accept and read the TD, but will reject the result if the Wash Buffer control does not yield a positive signal. In this latter embodiment, a wash/running buffer disposed in a compartment/sac disposed upstream of the sample can contain a control signal (e.g., label emitting at a different wavelength) which the reader is programmed to detect.

The signal obtained by the reader is processed using data processing software employing data reduction and curve fitting algorithms, optionally in combination with a trained neural network, to give either a positive or negative result for each test line, or a quantitative determination of the concentration of each analyte in the sample, which is correlated with a result indicative of a risk or presence of a disease or disorder. This result can optionally be input into a decision support system, and processed to provide an enhanced assessment of the risk of a medical condition as output. In one embodiment, the entire procedure may be automated and/or computer-controlled.

Multianalyte Point of Care System.

Rapid influenza tests have been marketed for years. Most of these tests are lateral flow immunoassay tests using either gold or latex as the visualization agent. While most of new rapid immunoassays are able to differentiate influenza Type A from influenza Type B, only few of them have both test lines for type A and type B on the one strip. However, none of these tests are designed to differentiate subtypes of influenza type A. Therefore, these tests may be able to detect avian influenza; however, none of them can tell if a patient is infected by a seasonal flu A virus or a more severe Type A subtype such as H5N1 termed avian influenza (or current potential pandemic subtype of influenza A). These tests can also detect swine influenza, such as type H1N1. The invention is designed on concepts that when applied are to yield a highly sensitive assay with improved reproducibility, able to detect type A, type B and differentiate subtype H5N1 or H1N1 from seasonal flu (subtypes H1 and H3) and is easy to use. Efforts, as described herein, have been made to apply multiple new technologies with a new device design, such as pre-mixing of the sample with the conjugate, use of a chasing or wash buffer to reduce background, employ a unique generic capture reagent pRNA which allows multiple analytes detection at high sensitivity, fluorescent label which is highly sensitive, etc. The combination of these approaches enables a novel and highly effective influenza rapid test that is much more sensitive, provides low cost production, ease of operate and has the ability to differentiate seasonal flu from pandemic avian flu H5N1 or swine flu H1N1 (e.g., 2009 H1N1).

Assay Methods.

In one embodiment, an assay method comprises the steps of applying the sampling implement to a subject or subject's biological sample, to collect a sample (e.g., swabbing inside the nose, mouth, throat, ear; applying a sampling element to a biological sample obtained from a subject), inserting the collection implement into the sample collection device housing chamber, applying a solution to the sample collection device (e.g., by squeezing the upper chamber to break open the snap-valve and allowing a buffer to run down to the sampling implement, thus immersing the biological sample disposed thereon) and running the mixture of buffer and sample into a mixing or reagent chamber (e.g., lower chamber) where a plurality of capture and detection probes bind to their specific target analyte. Subsequently or concurrently, the mixture is expelled from the distal end of the SCD into a TD comprising one or more immobilized partner capture moieties designed to capture a complex of analyte and detection/capture probe, via the complementary capture moiety linked to a capture probe. Thus, a particular capture probe for one particular analyte is designed to be complementary to an immobilized partner capture moiety. Furthermore, as disclosed herein, partner capture moieties are disposed on a test device (e.g., a lateral flow membrane) in distinct positions/patterns/zones, where a single line or spot(s) if detected via the signal emitting label, allows qualitative and/or quantitative detection of a particular analyte. Therefore, by patterning particular partner capture probes on the test device, an assay method can detect a panel of the same or related infectious agent(s) or even unrelated infectious agents, as disclosed herein.

In some embodiments, a sandwich immunoassay format is utilized but any conventional format, including a competitive assay, may be used. Examples of sandwich immunoassays performed on test strips are described in U.S. Pat. Nos. 4,168,146 and 4,366,241, each of which is incorporated herein by reference. Examples of competitive immunoassay devices are those disclosed by U.S. Pat. Nos. 4,235,601, 4,442,204 and 5,208,535, each of which is incorporated herein by reference. Some additional illustrative devices that can be adapted for competitive immunoassays include dipstick, lateral flow, cartridge, multiplexed, microtiter plate, microfluidic, plate or arrays or high throughput platforms, such as those disclosed in U.S. Pat. Nos. 6,448,001, 4,943,522, 6,485,982, 6,656,744, 6,811,971, 5,073,484, 5,716,778, 5,798,273, 6,565,808, 5,078,968, 5,415,994, 6,235,539, 6,267,722, 6,297,060, 7,098,040, 6,375,896, 7,083,912, 5,225,322, 6,780,582, 5,763,262, 6,306,642, 7,109,042, 5,952,173, and 5,914,241. Exemplary microfluidic devices include those disclosed in U.S. Pat. No. 5,707,799 and WO2004/029221.

In general, tracers used in such assays require either instrumentation and/or treatment of the tracer in order to determine the tracer in the bound and/or free portion of the assay as a measure of analyte. For example, in an assay in which an enzyme is used as the label or marker for the tracer, the enzyme must be developed with a suitable developer. When the label or marker is a fluorescent material, the tracer in the bound and/or free portion is determined by the use of appropriate instrumentation for determining fluorescence.

Alternatively a tracer used in the assay is a ligand labeled with a particulate label which is visible when bound to the binder on the support or when bound to the analyte bound to the binder on the support, without further treatment, and wherein the ligand is bound by either the binder or analyte. See also U.S. Pat. No. 4,703,017, which is incorporated herein by reference.

In another particular aspect, a non-nucleic acid based screening test includes any solid phase, lateral flow, or flow-through tests. In general, solid phase immunoassay devices incorporate a solid support to which one member of a ligand-receptor pair, usually an antibody, antigen, or hapten, is bound. Common early forms of solid supports were plates, tubes, or beads of polystyrene, which were known from the fields of radioimmunoassay and enzyme immunoassay. More recently, a number of porous materials such as nylon, nitrocellulose, cellulose acetate, glass fibers, and other porous polymers have been employed as solid supports

In one embodiment, a sample is collected from a subject via a sampling implement and placed back into the cylinder housing of the SCD device. The SCD can first be inserted into a TD, or prior to insertion into a TD, a solution contained in the upper chamber of the SCD is released to effect washing the sample and solution into a mixing or reagent chamber. Either liquid or solid reagents comprising detection and capture probes that target one or more different analytes as disclosed herein can be present in the mixing or reagent chamber. Upon mixing a complex of analyte bound to detection and capture probe is formed if analyte is present. The sample is then expelled from the SCD into a TD through an aperture that seals the contact between the SCD and the TD from the outside environment (e.g., preventing any spillage, aerosol or contamination). The sample mixture can flow as a result of gravity or the force of air pressure in the SCD (e.g., squeezing an upper sealed chamber) into a TD. The sample is driven by capillary force and/or by buffer present in the TD so as to allow any analyte-probe complex to pass through a detection zone (e.g., on a lateral flow membrane) contained in the TD. Capture probes and complementary immobilized partner capture moieties bind or hybridize to each other (e.g., in predetermined lines or spots on the lateral flow membrane), whereby detection probes (via conjugate labels contained thereon) will provide a detectable signal which can subsequently be read to determine which analytes were present in the sample processed.

In one embodiment, TDs with samples processed thereon, can be set aside for time periods of about 1, 2, 3, 4, 5, 6 or 8 hours before reading the results, and yet provide results as accurately as if read in 15 or 20 minutes after processing. Thus, the signals produced are stable for long periods of time so that reading the results may occur at a significantly later time after the tests are actually performed. This is a great improvement for point-of-care diagnostics, where in the field conditions often present limited resources in manpower and time, and where the test setting can be in remote regions that are not easily or quickly accessed.

Binding Reagents.

One aspect of the invention is directed to an SCD of the invention comprising a plurality of different Analyte Binding Sets, wherein each particular Analyte Binding Set is configured to bind the same target analyte, and wherein different Analyte Binding Sets are provided so as to binding and detect different target analytes. For example, an SCD can comprise one, two, three, four, five or more Analyte Binding Sets, wherein each set is specific for a different target analyte as compared to any other set present in the SCD. Therefore, an Analyte Binding Set targeting the same target analyte comprises: (1) a capture probe comprising: (i) an specific binding agent that binds a target analyte and (ii) a capture moiety partner (e.g., a pRNA), and (2) a detection probe. A “detection probe” (also may be referred to as a “label probe”) is also capable of binding the same target analyte and is linked to a detectable label.

In one embodiment, the capture moiety partner of a capture probe targeting conjugate is capable of binding to an immobilized binding partner, for example, a binding partner present on a lateral flow membrane in a test device.

In one embodiment, a detection probe comprises a analyte-specific binding agent that is bound (directly or indirectly) to a detectable label, and upon contacting with a sample containing the target analyte forms a complex with the target analyte. Furthermore, the capture probe would similarly bind the same target analyte thus forming a detection probe-target analyte-capture probe complex. Such a complex can then be immobilized (“captured”) on a solid support via an immobilized capture moiety partner that is capable of specifically binding to the CMP present on the capture probe. The resulting complex is immobilized on the solid support and is detected by virtue of the detectable label.

In one embodiment, a SCD comprises a plurality of different Analyte Binding Sets wherein each set comprises detection probes and capture probes that are capable of binding a target analyte, which includes an infectious agent, a disease causing microorganism or components thereof (e.g., antigen, polypeptide, nucleic acid).

In various embodiments, a TD comprises one or more addressable lines (or test zone) discretely positioned on a test substrate, wherein each test zone is configured for detection of a different type of infectious agent or disease causing micro-organism or component therefrom.

In another embodiment, one or more test zones are configured for detection of one or more different types or subtypes of the same infectious agent. As used herein in the context of a test zone the term “configured” means that ICMPs in any one addressable line are capable of specifically binding cognate CMPs present in detection probes of an Analyte Binding Set that is designed to bind the target analyte for the test zone.

In one embodiment, a TD comprises a plurality of addressable lines, wherein at least two adjacent addressable lines comprise a different category of CMP. In another embodiment, a TD comprises a plurality of addressable lines wherein at least two addressable lines comprise CMPs that are pRNA, and wherein at least one addressable line comprises an avidin or streptavidin. For example, pRNAs would be the same type or category of CMP, while pRNA and avidin/biotin would represent different categories of CMP. Other categories of CMPs can be utilized, including other specific binding partners, such as, antigen/antibody pairs, where the antigen is distinct from the analytes of interest.

In one embodiment, a test strip also comprises one or more addressable lines that function as a control line to determine that an assay is functioning properly. In one embodiment, a control line has disposed thereon an antibody that will specifically bind to the analyte-specific binding agent comprised in a capture probe. In one example, an antibody disposed on a control line is rabbit anti-mouse antibody, where the antibody in the capture probe is a mouse antibody prepared against the analyte of interest.

In some embodiments, an Analyte Binding Set comprises an antibody pair, where each antibody member of the pair can specifically bind the same target analyte, wherein one antibody is a targeting antibody in the capture probe and the other is a detection antibody in the detection probe, where each antibody binds to a different epitope of the antigen and thus each is capable of binding the same analyte/antigen at the same time to form a “sandwich”.

In addition to antigen and antibody specific binding pair members, other specific binding pairs include, as examples without limitation, biotin and avidin, carbohydrates and lectins, complementary nucleotide sequences, complementary peptide sequences, effector and receptor molecules, enzyme cofactors and enzymes, enzyme inhibitors and enzymes, a peptide sequence or chemical moiety (such as digoxin/anti-digoxin) and an antibody specific for the sequence, chemical moiety or the entire protein, polymeric acids and bases, dyes and protein binders, peptides and specific protein binders (e.g., ribonuclease, S-peptide and ribonuclease S-protein), metals and their chelators, and the like. Furthermore, specific binding pairs can include members that are analogs of the original specific binding member, for example an analyte-analog or a specific binding member made by recombinant techniques or molecular engineering.

Antibodies.

In various embodiments, the specific binding agent of the capture probes and detection probes of the invention comprise a target analyte-specific binding moiety that can be an antibody or functional fragment thereof.

In other embodiments, an ICMP is an antibody that is specific for an antigen that is then utilized as a component of a capture probe, wherein the antigen functions as a cognate CMP for the immobilized antibody.

If an antibody is used, it can be a monoclonal or polyclonal antibody, a recombinant protein or antibody, a chimeric antibody, a mixture(s) or fragment(s) thereof, as well as a mixture of an antibody and other specific binding members. Other examples of binding pairs that can be incorporated into the detection molecules are disclosed in, for example, U.S. Pat. Nos. 6,946,546, 6,967,250, 6,984,491, 7,022,492, 7,026,120, 7,022,529, 7,026,135, 7,033,781, 7,052,854, 7,052,916 and 7,056,679.

“Antibody” refers to a polypeptide substantially encoded by an immunoglobulin gene or immunoglobulin genes, or fragments thereof, and includes any immunoglobulin, including monoclonal antibodies, polyclonal antibodies, multispecific or bispecific antibodies, that bind to a specific antigen. A complete antibody comprises two heavy chains and two light chains. Each heavy chain consists of a variable region and a first, second, and third constant region, while each light chain consists of a variable region and a constant region. The antibody has a “Y” shape, with the stem of the Y consisting of the second and third constant regions of two heavy chains bound together via disulfide bonding. Each arm of the Y consists of the variable region and first constant region of a single heavy chain bound to the variable and constant regions of a single light chain. The variable regions of the light and heavy chains are responsible for antigen binding. The variable region in both chains generally contains three highly variable loops called the complementarity determining regions (CDRs) (light (L) chain CDRs including LCDR1, LCDR2, and LCDR3, heavy (H) chain CDRs including HCDR1, HCDR2, HCDR3) (as defined by Kabat, et al., Sequences of Proteins of Immunological Interest, Fifth Edition (1991), vols. 1-3, NIH Publication 91-3242, Bethesda Md.). The three CDRs are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. The recognized immunoglobulin genes include the kappa, lambda, alpha, gamma, delta, epsilon, and mu constant regions, as well as myriad immunoglobulin variable region genes. Light chains are classified as either kappa or lambda. Heavy chains are classified as gamma, mu, alpha, delta, or epsilon, which in turn define the immunoglobulin classes and subclasses include IgG, IgG1, IgG2, IgG3, IgG4, IgM, IgA, IgA1, or IgA2, IgD, and IgE, respectively. Typically, an antibody is an immunoglobulin having an area on its surface or in a cavity that specifically binds to and is thereby defied as complementary with a particular spatial and polar organization of another molecule. The antibody can be polyclonal or monoclonal. Antibodies may include a complete immunoglobulin or fragments thereof. Fragments thereof may include Fab, Fv and F(ab′)2, Fab′, and the like. Antibodies may also include chimeric antibodies or fragment thereof made by recombinant methods. Antibodies are assigned to classes based on the amino acid sequence of the constant region of their heavy chain. The major classes of antibodies are IgA, IgD, IgE, IgG, and IgM, with several of these classes divided into subclasses such as.

In addition to an intact immunoglobulin, the term “antibody” as used herein further refers to an immunoglobulin fragment thereof (i.e., at least one immunologically active portion of an immunoglobulin molecule), such as a Fab, Fab′, F(ab′)₂, Fv fragment, a single-chain antibody molecule, a multispecific antibody formed from any fragment of an immunoglobulin molecule comprising one or more CDRs. In addition, an antibody as used herein may comprise one or more CDRs from a particular human immunoglobulin grafted to a framework region from one or more different human immunoglobulins.

“Fab” with regards to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“Fc” with regards to an antibody refers to that portion of the antibody consisting of the second and third constant regions of a first heavy chain bound to the second and third constant regions of a second heavy chain via disulfide bonding. The Fc portion of the antibody is responsible for various effector functions but does not function in antigen binding.

“Fv” with regards to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Houston 1988).

“Single-chain Fv-Fc antibody” or “scFv-Fc” refers to an engineered antibody consisting of a scFv connected to the Fc region of an antibody.

The term “epitope” as used herein refers to the group of atoms and/or amino acids on an antigen molecule to which an antibody binds.

The term “monoclonal antibody” as used herein refers to an antibody or a fragment thereof obtained from a population of substantially homogeneous antibodies, i.e., the individual antibodies comprising the population are identical except for possible naturally occurring mutations that may be present in minor amounts. Monoclonal antibodies are highly specific, being directed against a single epitope on the antigen. Monoclonal antibodies are in contrast to polyclonal antibodies which typically include different antibodies directed against different epitopes on the antigens. Although monoclonal antibodies are traditionally derived from hybridomas, monoclonal antibodies are not limited by their production method. For example, monoclonal antibodies can be made by the hybridoma method first described by Kohler et al., Nature, 256:495 (1975), or may be made by recombinant DNA methods (see, e.g., U.S. Pat. No. 4,816,567).

The term “chimeric antibody” as used herein refers to an antibody in which a portion of the heavy and/or light chain is identical with or homologous to corresponding sequences in antibodies derived from a particular species or belonging to a particular antibody class or subclass, while the remainder of the heavy and/or light chain is identical with or homologous to corresponding sequences in antibodies derived from another species or belonging to another antibody class or subclass, as well as fragments of such an antibody, so long as such fragments exhibit the desired antigen-binding activity (U.S. Pat. No. 4,816,567 to Cabilly et al.; Morrison et al., Proc. Natl. Acad. Sci. USA, 81:6851 6855 (1984)).

The term “humanized antibody” used herein refers to an antibody or fragments thereof which are human immunoglobulins (recipient antibody) in which residues from part or all of a CDR of the recipient are replaced by residues from a CDR of a non-human species (donor antibody) such as mouse, rat or rabbit having the desired specificity, affinity, and capacity. In some instances, FR residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin Fc region, typically that of a human immunoglobulin. For further details, see Jones et al., Nature, 321:522 525 (1986); Reichmann et al., Nature, 332:323 329 (1988); Presta, Curr. Op. Struct. Biol., 2:593 596 (1992); and Clark, Immunol. Today 21: 397 402 (2000).

In some embodiments, anti-H5 monoclonal antibodies are produced by mice hybridoma cell strains 8H5, 3C8, 10F7, 4D1, 3G4 and 2F2. These monoclonal antibodies are named after the hybridoma cell strains that produce them. Thus the anti-H5 monoclonal antibodies that are produced by mice hybridoma cell strains 8H5, 3C8, 10F7, 4D1, 3G4, and 2F2, respectively, are named monoclonal antibodies 8H5, 3C8, 10F7, 4D1, 3G4, and 2F2, respectively. Monoclonal antibodies 8H5, 3C8, 10F7, 4D1, 3G4, and 2F2 specifically bind to the hemagglutinin of subtype H5 avian influenza virus. The mice hybridoma cell strains 8H5, 3C8, 10F7, 4D1, 3G4, and 2F2 were deposited in China Center for Typical Culture Collection (CCTCC, Wuhan University, Wuhan, China) on Jan. 17, 2006 with deposit numbers of CCTCC-C200607 (hybridoma cell strain 8H5), CCTCC-C200605 (hybridoma cell strain 3C8), CCTCC-C200608 (hybridoma cell strain 10F7), CCTCC-C200606 (hybridoma cell strain 4D1), CCTCC-C200604 (hybridoma cell strain 3G4) and CCTCC-C200424 (hybridoma cell strain 2F2).

In various embodiment, monoclonal antibodies are provided that block the binding of monoclonal antibodies 8H5, 3C8, 10F7, 4D1, 3G4, or 2F2 to the hemagglutinin of subtype H5 avian influenza virus. Such blocking monoclonal antibodies may bind to the same epitopes on the hemagglutinin that are recognized by monoclonal antibodies 8H5, 3C8, 10F7, 4D1, 3G4, or 2F2. Alternatively, those blocking monoclonal antibodies may bind to epitopes that overlap sterically with the epitopes recognized by monoclonal antibodies 8H5, 3C8, 10F7, 4D1, 3G4, or 2F2. These blocking monoclonal antibodies can reduce the binding of monoclonal antibodies 8H5, 3C8, 10F7, 4D1, 3G4, or 2F2 to the hemagglutinin of subtype H5 avian influenza virus by at least about 50%. Alternatively, they may reduce binding by at least about 60%, preferably at least about 70%, more preferably at least about 75%, more preferably at least about 80%, more preferably at least about 85%, even more preferably at least about 90%, even more preferably at least about 95%, most preferably at least about 99%.

The ability of a test monoclonal antibody to reduce the binding of a known monoclonal antibody to the H5 hemagglutinin may be measured by a routine competition assay such as that described in Antibodies: A Laboratory Manual, Cold Spring Harbor Laboratory, Ed Harlow and David Lane (1988). For example, such an assay could be performed by pre-coating a microtiter plate with antigens, incubating the pre-coated plates with serial dilutions of the unlabeled test antibodies admixed with a selected concentration of the labeled known antibodies, washing the incubation mixture, and detecting and measuring the amount of the known antibodies bound to the plates at the various dilutions of the test antibodies. The stronger the test antibodies compete with the known antibodies for binding to the antigens, the more the binding of the known antibodies to the antigens would be reduced. Usually, the antigens are pre-coated on a 96-well plate, and the ability of unlabeled antibodies to block the binding of labeled antibodies is measured using radioactive or enzyme labels.

Monoclonal antibodies may be generated by the hybridoma method first described by Kohler et al., Nature, 256: 495 (1975). In the hybridoma method, a mouse or other appropriate host animal is immunized by one or more injections of an immunizing agent and, if desired, an adjuvant. Typically, the immunizing agent and/or adjuvant will be injected in the host animal by multiple subcutaneous or intraperitoneal injections. It may be useful to conjugate the immunizing agent to a protein known to be immunogenic in the host animal being immunized, such as serum albumin, or soybean trypsin inhibitor. Examples of adjuvants which may be employed include Freund's complete adjuvant and MPL-TDM. After immunization, the host animal makes lymphocytes that produce or are capable of producing antibodies that will specifically bind to the antigen used for immunization. Alternatively, lymphocytes may be immunized in vitro. Desired lymphocytes are collected and fused with myeloma cells using a suitable fusing agent, such as polyethylene glycol, to form a hybridoma cell (Goding, Monoclonal Antibodies: Principles and Practice, pp. 59 103, Academic Press, 1996).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and MC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63, Marcel Dekker, Inc., New York, 1987).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis of Munson et al., Anal. Biochem., 107: 220 (1980).

After hybridoma cells are identified that produce antibodies of the desired specificity, affinity, and/or activity, the cells may be subcloned by limiting dilution procedures and grown by standard methods (Goding, Monoclonal Antibodies: Principles and Practice, pp. 59-103, Academic Press, 1996). Suitable culture media for this purpose include, for example, DMEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies of the invention may also be made by conventional genetic engineering methods. DNA molecules encoding the heavy and light chains of the monoclonal antibodies may be isolated from the hybridoma cells, for example through PCR using oligonucleotide probes that are capable of binding specifically to genes encoding the heavy and light chains of the monoclonal antibodies. Then the DNA molecules are inserted into expression vectors. The expression vectors are transfected into host cells such as E. coli cells, simian COS cells, Chinese hamster ovary (CHO) cells, or myeloma cells that do not otherwise produce immunoglobulin protein. The host cells are cultured under conditions suitable for the expression of the antibodies.

The antibodies of the invention can bind to the H5 hemagglutinin with high specificity and affinity. The antibodies shall have low cross-reactivity with other subtypes of hemagglutinin, preferably no cross-reactivity with other subtypes of hemagglutinins. In one aspect, the invention provides antibodies that bind to H5 hemagglutinin with a K_{D} value of less than 1×10⁻⁵M. Preferably, the K_{D} value is less than 1×10⁻⁶M. More preferably, the K_{D} value is less than 1×10⁻⁷M. Most preferably, the K_{D} value is less than 1×10⁻⁸M.

The antibodies of the invention may contain the conventional “Y” shape structure comprised of two heavy chains and two light chains. In addition, the antibodies may also be the Fab fragment, the Fab′ fragment, the F(ab)₂ fragment or the Fv fragment, or another partial piece of the conventional “Y” shaped structure that maintains binding affinity to the hemagglutinin. The binding affinity of the fragments to hemagglutinin may be higher or lower than that of the conventional “Y” shaped antibodies.

The antibody fragments may be generated via proteolytic digestion of intact antibodies (see, e.g., Morimoto et al., J. Biochem. Biophys. Methods, 24:107-117, (1992) and Brennan et al., Science, 229:81 (1985)). Additionally, these fragments can also be produced directly by recombinant host cells (reviewed in Hudson, Curr. Opin. Immunol., 11: 548-557 (1999); Little et al., Immunol. Today, 21: 364-370 (2000)). For example, Fab′ fragments can be directly recovered from E. coli and chemically coupled to form F(ab′)₂ fragments (Carter et al., Bio/Technology, 10:163 167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. Other techniques for the production of antibody fragments will be apparent to a person with ordinary skill in the art.

In some embodiments, isolated nucleic acid molecules encoding antibodies or fragments specifically bind to H5 hemagglutinin. Nucleic acid molecules encoding the antibodies can be isolated from hybridoma cells. The nucleic acid sequences of the molecules can be determined using routine techniques known to a person with ordinary skill in the art. Nucleic acid molecules of the invention can also be prepared using conventional genetic engineering techniques as well as chemical synthesis. In one embodiment, an isolated nucleic acid molecule encodes the variable region of the heavy chain of an anti-H5 (HA) antibody or a portion of the nucleic acid molecule. In another embodiment, an isolated nucleic acid molecule encodes the variable region of the light chain of an anti-H5 (HA) antibody or a portion of the nucleic acid molecule. In another aspect, an isolated nucleic acid molecule encodes the CDRs of the antibody heavy chain or light chain variable regions.

In one embodiment, isolated nucleic acid molecules encode the variable regions of the heavy chain and light chain of monoclonal antibodies 8H5, 3C8, 10F7, 4D1, 3G4, and 2F2. The nucleic acid sequences encoding the heavy chain variable regions of monoclonal antibodies 8H5, 3C8, 10F7, 4D1, 3G4, and 2F2 are set forth in SEQ ID NO: 1, SEQ ID NO: 5, SEQ ID NO: 9, SEQ ID NO:16, SEQ ID NO:20 and SEQ ID NO: 24, respectively. The nucleic acid sequences encoding the light chain variable regions of monoclonal antibodies 8H5, 3C8, 10F7, 4D1, and 2F2 are set forth in SEQ ID NO: 3, SEQ ID NO: 7, SEQ ID NO: 11, SEQ ID NO:18, SEQ ID NO: 26, respectively. In some embodiments, degenerative analogs of the nucleic acid molecules encode the variable regions of the heavy chain and light chain of monoclonal antibodies 8H5, 3C8, 10F7, 4D1, 3G4 and 2F2.

In another embodiment, isolated nucleic acid variants share sequence identity with the nucleic acid sequences of SEQ ID NO: 1, SEQ ID NO: 3, SEQ ID NO: 5, SEQ ID NO: 7, SEQ ID NO: 9, SEQ ID NO: 11, SEQ ID NO: 16, SEQ ID NO:18, SEQ ID NO:20, SEQ ID NO:24 or SEQ ID NO:26. In one embodiment, the nucleic acid variants share at least 70% sequence identity, preferably at least 75% sequence identity, more preferably at least 80% sequence identity, more preferably at least 85% sequence identity, more preferably at least 90% sequence identity, most preferably at least 95% sequence identity, to the sequences of SEQ ID NO: 1, SEQ ID NO: 3, SEQ ID NO: 5, SEQ ID NO: 7, SEQ ID NO: 9, SEQ ID NO: 11, SEQ ID NO:16, SEQ ID NO:18, SEQ ID NO:20, SEQ ID NO:24 or SEQ ID NO:26.

In some embodiments, isolated nucleic acid molecules encoding antibody fragments are capable of specifically binding to subtype H5 of avian influenza virus.

In some embodiments, isolated nucleic acid molecules encoding an antibody heavy chain variable region comprise the amino acid sequence set forth in SEQ ID NOs: 28-30, SEQ ID NOs: 34-36, SEQ ID NOs: 40-42, SEQ ID NOs: 46-48; SEQ ID NOs: 52-54, and SEQ ID NOs: 58-60. In some embodiments, isolated nucleic acid molecules encode an antibody light chain variable region comprising the amino acid sequence set forth in SEQ ID NOs: 31-33, SEQ ID NOs: 37-39, SEQ ID NOs: 43-45, SEQ ID NOs: 49-51, SEQ ID NOs: 55-57, and SEQ ID NOs: 61-63.

In some embodiments, recombinant expressing vectors comprise the isolated nucleic acid molecules of the invention. It also provides host cells transformed with the nucleic acid molecules. One aspect of the invention is a method of producing antibodies of the invention comprising culturing the host cells under conditions wherein the nucleic acid molecules are expressed to produce the antibodies and isolating the antibodies from the host cells.

Antibody Polypeptide Sequences

The amino acid sequences of the variable regions of the heavy chain and light chain of monoclonal antibodies 8H5, 3C8, 10F7, 4D1, 3G4 and 2F2 have been deduced from their respective nucleic acid sequences. The amino acid sequences of the heavy chain variable regions of monoclonal antibodies 8H5, 3C8, 10F7, 4D1, 3G4 and 2F2 are set forth in SEQ ID NO:2, SEQ ID NO:6, SEQ ID NO: 10, SEQ ID NO:17, SEQ ID NO:21, and SEQ ID NO:25, respectively. The amino acid sequences of the light chain variable regions of monoclonal antibodies 8H5, 3C8, 10F7, 4D1, and 2F2 are set forth in SEQ ID NO:4, SEQ ID NO:8, SEQ ID NO:12, SEQ ID NO:19, and SEQ ID NO:27. In one aspect, anti-H5 antibodies comprise a heavy chain variable region comprising the amino acid sequences as set forth in SEQ ID NO:2, SEQ ID NO:6, SEQ ID NO: 10, SEQ ID NO:17, SEQ ID NO:21, and SEQ ID NO:25. In another aspect, anti-H5 antibodies comprise a light chain variable region comprising the amino acid sequences as set forth in SEQ ID NO:4, SEQ ID NO:8, SEQ ID NO:12, SEQ ID NO:19, and SEQ ID NO:27.

In another aspect, an antibody heavy chain comprises a variable region having at least 70% sequence identity, preferably at least 75% sequence identity, more preferably at least 80% sequence identity, more preferably at least 85% sequence identity, more preferably at least 90% sequence identity, most preferably at least 95% sequence identity to the amino acid sequences set forth in SEQ ID NO:2, SEQ ID NO:6, SEQ ID NO: 10, SEQ ID NO:17, SEQ ID NO:21, and SEQ ID NO:25.

In another aspect, an antibody light chain comprises a variable region having at least 70% sequence identity, preferably at least 75% sequence identity, more preferably at least 80% sequence identity, more preferably at least 85% sequence identity, more preferably at least 90% sequence identity, most preferably at least 95% sequence identity to the amino acid sequences set forth in SEQ ID NO:4, SEQ ID NO:8, SEQ ID NO:12, SEQ ID NO:19, and SEQ ID NO:27.

The amino acid sequences of the CDRs of the variable regions of the heavy chain and light chain of monoclonal antibodies 8H5, 3C8, 10F7, 4D1, 3G4, and 2F2 have also been determined as follows:

The amino acid sequences of CDR1, CDR2 and CDR3 of the heavy chain of monoclonal antibody 8H5 are set forth in SEQ ID Nos:28-30, respectively. The amino acid sequences of CDR1, CDR2 and CDR3 of the light chain of monoclonal antibody 8H5 are set forth in SEQ ID Nos:31-33, respectively.

The amino acid sequences of CDR1, CDR2 and CDR3 of the heavy chain of monoclonal antibody 3C8 are set forth in SEQ ID Nos:34-36, respectively. The amino acid sequences of CDR1, CDR2 and CDR3 of the light chain of monoclonal antibody 3C8 are set forth in SEQ ID Nos:37-39, respectively.

The amino acid sequences of CDR1, CDR2 and CDR3 of the heavy chain of monoclonal antibody 10F7 are set forth in SEQ ID Nos:40-42, respectively. The amino acid sequences of CDR1, CDR2 and CDR3 of the light chain of monoclonal antibody 10F7 are set forth in SEQ ID Nos:43-45, respectively.

The amino acid sequences of CDR1, CDR2 and CDR3 of the heavy chain of monoclonal antibody 4D1 are set forth in SEQ ID Nos:46-48, respectively. The amino acid sequences of CDR1, CDR2 and CDR3 of the light chain of monoclonal antibody 4D1 are set forth in SEQ ID Nos:49-51, respectively.

The amino acid sequences of CDR1, CDR2 and CDR3 of the heavy chain of monoclonal antibody 3G4 are set forth in SEQ ID Nos:52-54, respectively. The amino acid sequences of CDR1, CDR2 and CDR3 of the light chain of monoclonal antibody 3G4 are set forth in SEQ ID Nos:55-57, respectively.

The amino acid sequences of CDR1, CDR2 and CDR3 of the heavy chain of monoclonal antibody 2F2 are set forth in SEQ ID Nos:58-60, respectively. The amino acid sequences of CDR1, CDR2 and CDR3 of the light chain of monoclonal antibody 2F2 are set forth in SEQ ID Nos:61-63, respectively.

TABLE 1
Six strains of monoclonal antibody CDRs amino acid sequence.

| Monoclonal | Antibody heavy chain | | | Antibody light chain | | |
|---|---|---|---|---|---|---|
| antibody | CDRs amino acid sequence | | | CDRs amino acid sequence | | |
| strains | CDR1 | CDR2 | CDR3 | CDR1 | CDR2 | CDR3 |
| 8H5 | GYTFSNYW | ILPGSDRT | ANRYDGYYFGLDY | SSVNF | YSS | QHFTSSPYT |
| | (SEQ ID | (SEQ ID | (SEQ ID | (SEQ ID | (SEQ ID | (SEQ ID |
| | NO: 28) | NO: 29) | NO: 30) | NO: 31) | NO: 32) | NO: 33) |
| 3C8 | GYSFTNYG | INTHTGEP | ARWNRDAMDY | ESVDSSDNSL | RAS | QQSIGDPPYT |
| | (SEQ ID | (SEQ ID | (SEQ ID | (SEQ ID | (SEQ ID | (SEQ ID |
| | NO: 34) | NO: 35) | NO: 36) | NO: 37) | NO: 38) | NO: 39) |
| 10F7 | GYTFTSYW | IDPSDSYT | ARGGTGDFHYAMDY | QGISSN | HGT | QYVQFPYT |
| | (SEQ ID | (SEQ ID | (SEQ ID | (SEQ ID | (SEQ ID | (SEQ ID |
| | NO: 40) | NO: 41) | NO: 42) | NO: 43) | NO: 44) | NO: 45) |
| 4D1 | GYTFTSYW | IDPSDSFT | ARGGPGDFRYAMDY | QGISSN | HGT | VQYVQFPYT |
| | (SEQ ID | (SEQ ID | (SEQ ID | (SEQ ID | (SEQ ID | (SEQ ID |
| | NO: 46) | NO: 47) | NO: 48) | NO: 49) | NO: 50) | NO: 51) |
| 3G4 | GYTFTDYA | INTDYGDT | ARSDYDYYFCGMDY | (SEQ ID | (SEQ ID | (SEQ ID |
| | (SEQ ID | (SEQ ID | (SEQ ID | NO: 55) | NO: 56) | NO: 57) |
| | NO: 52) | NO: 53) | NO: 54) | | | |
| 2F2 | GFSLTGYG | IWAEGRT | AREVITTEAWYFDV | QSISDY | YAS | QNGHTFPLT |
| | (SEQ ID | (SEQ ID | (SEQ ID | (SEQ ID | (SEQ ID | (SEQ ID |
| | NO: 58) | NO: 59) | NO: 60) | NO: 61) | NO: 62) | NO: 63) |

In another aspect, an anti-H5 monoclonal antibody heavy chain or a fragment thereof, comprises the following CDRs: (i) one or more CDRs selected from SEQ ID NOs: 28-30; (ii) one or more CDRs selected from SEQ ID NOs: 34-36; (iii) one or more CDRs selected from SEQ ID NOs: 40-42; (iv) one or more CDRs selected from SEQ ID NOs: 46-48; (v) one or more CDRs selected from SEQ ID NOs: 52-54; or (vi) one or more CDRs selected from SEQ ID NOs: 58-60. In one embodiment, the anti-H5 monoclonal antibody heavy chain or a fragment thereof comprises three CDRs having the amino acid sequences set forth in SEQ ID NOs: 28-30, respectively. In another embodiment, the anti-H5 monoclonal antibody heavy chain or a fragment thereof comprises three CDRs having the amino acid sequences set forth in SEQ ID NOs: 34-36, respectively. In another embodiment, the anti-H5 monoclonal antibody heavy chain or a fragment thereof comprises three CDRs having the amino acid sequences set forth in SEQ ID NOs: 40-42. In another embodiment, the anti-H5 monoclonal antibody heavy chain or a fragment thereof comprises three CDRs having the amino acid sequences set forth in SEQ ID NOs: 46-48. In another embodiment, the anti-H5 monoclonal antibody heavy chain or a fragment thereof comprises three CDRs having the amino acid sequences set forth in SEQ ID NOs: 52-54. In another embodiment, the anti-H5 monoclonal antibody heavy chain or a fragment thereof comprises three CDRs having the amino acid sequences set forth in SEQ ID NOs: 58-60.

In another aspect, the CDRs contained in the anti-H5 monoclonal antibody heavy chains or fragments thereof can include one or more amino acid substitution, addition and/or deletion from the amino acid sequences set forth in SEQ ID NOs: 28-30, 34-36, 40-42, 46-48, 52-54, and 58-60. Preferably, the amino acid substitution, addition and/or deletion occur at no more than three amino acid positions. More preferably, the amino acid substitution, addition and/or deletion occur at no more than two amino acid positions. Most preferably, the amino acid substitution, addition and/or deletion occur at no more than one amino acid position.

In another aspect, an anti-H5 monoclonal antibody light chain or a fragment thereof comprises the following CDRs: (i) one or more CDRs selected from SEQ ID NOs: 31-33; (ii) one or more CDRs selected from SEQ ID NOs: 37-39; (iii) one or more CDRs selected from SEQ ID NOs: 43-45; (iv) one or more CDRs selected from SEQ ID NOs: 49-51; (v) one or more CDRs selected from SEQ ID NOs: 55-57; or (vi) one or more CDRs selected from SEQ ID NOs: 61-63. In one embodiment, the anti-H5 monoclonal antibody light chain or a fragment thereof comprises three CDRs having the amino acid sequences set forth in SEQ ID NOs: 31-33, respectively. In another embodiment, the anti-H5 monoclonal antibody light chain or a fragment thereof comprises three CDRs having the amino acid sequences set forth in SEQ ID NOs: 37-39, respectively. In another embodiment, the anti-H5 monoclonal antibody light chain or a fragment thereof comprises three CDRs having the amino acid sequences set forth in SEQ ID NOs: 43-45. In another embodiment, the anti-H5 monoclonal antibody light chain or a fragment thereof comprises three CDRs having the amino acid sequences set forth in SEQ ID NOs: 49-51. In another embodiment, the anti-H5 monoclonal antibody light chain or a fragment thereof comprises three CDRs having the amino acid sequences set forth in SEQ ID NOs: 55-57. In another embodiment, the anti-H5 monoclonal antibody light chain or a fragment thereof comprises three CDRs having the amino acid sequences set forth in SEQ ID NOs: 61-63.

In another aspect, the CDRs contained in the anti-H5 monoclonal antibody light chains or fragments thereof can include one or more amino acid substitution, addition and/or deletion from the amino acid sequences set forth in SEQ ID NOs: 31-33, 37-39, 43-45, 49-51, 55-57, and 61-63. Preferably, the amino acid substitution, addition and/or deletion occur at no more than three amino acid positions. More preferably, the amino acid substitution, addition and/or deletion occur at no more than two amino acid positions. Most preferably, the amino acid substitution, addition and/or deletion occur at no more than one amino acid position.

TABLE 2
The Amino Acid Sequences of the 7aa peptides
that bind to 8H5 mAb or 3C8 mAb.

| Monoclonal | 7 peptide | |
|---|---|---|
| Antibody | sequences | Sequence No. |
| 8H5 | H G M L P V Y | SEQ ID No: 64 |
| | P P S N Y G R | SEQ ID No: 65 |
| | P P S N F G K | SEQ ID No: 66 |
| | G D P W F T S | SEQ ID No: 67 |
| | N S G P W L T | SEQ ID No: 68 |
| 3C8 | W P P L S K K | SEQ ID No: 70 |
| | N T F R T P I | SEQ ID No: 71 |
| | N T F R D P N | SEQ ID No: 72 |
| | N P I W T K L | SEQ ID No: 73 |

The variants generated by amino acid substitution, addition and/or deletion in the variable regions of the above described antibodies or the above described CDRs maintain the ability of specifically binding to subtype H5 of avian influenza virus. Some embodiments also include antigen-binding fragments of such variants.

Monoclonal antibody variants of the invention may be made by conventional genetic engineering methods. Nucleic acid mutations may be introduced into the DNA molecules using methods known to a person with ordinary skill in the art. Alternately, the nucleic acid molecules encoding the heavy and light chain variants may be made by chemical synthesis.

In another aspect, the screening method of the invention comprises the steps of (i) culturing a peptide display library under conditions suitable for peptide expression; (ii) contacting the culture solution with monoclonal antibodies of the invention; (iii) selecting the phage clones that specifically bind to said monoclonal antibodies. The monoclonal antibodies used for the screening may include without limitation the monoclonal antibodies 8H5, 3C8, 10F7, 4D1 and 3G4.

TABLE 3
The sequences of the 12aa peptides
that bind to 8H5 mAb.

| Peptide | | |
|---|---|---|
| section | Amino Acid | |
| No. | Sequence | Base Sequence |
| 121 | MEPVKKYPTRSP | ATGGAGCCGGTGAAGAAGTATCCG |
| | (SEQ ID NO: 74) | ACGCGTTCTCCT |
| | | (SEQ ID NO: 75) |
| 122 | ETQLTTAGLRLL | GAGACTCAGCTGACTACGGCGGGT |
| | (SEQ ID NO: 76) | CTTCGGCTGCTT |
| | | (SEQ ID NO: 77) |
| 123 | ETPLTETALKWH | GAGACGCCTCTTACGGAGACGGCT |
| | (SEQ ID NO: 78) | TTGAAGTGGCAT |
| | | (SEQ ID NO: 79) |
| 124 | QTPLTMAALELF | CAGACGCCGCTGACTATGGCTGCT |
| | (SEQ ID NO: 80) | CTTGAGCTTTTT |
| | | (SEQ ID NO: 81) |
| 125 | DTPLTTAALRLV | GATACTCCGCTGACGACGGCGGCT |
| | (SEQ ID NO: 82) | CTTCGGCTGGTT |
| | | (SEQ ID NO: 83) |
| 126 | TPLTLWALSGLR | ACGCCGCTTACGCTTTGGGCTCTT |
| | (SEQ ID NO: 84) | TCTGGGCTGAGG |
| | | (SEQ ID NO: 85) |
| 128 | QTPLTETALKWH | CAGACGCCTCTTACGGAGACGGCT |
| | (SEQ ID NO: 86) | TTGAAGTGGCAT |
| | | (SEQ ID NO: 87) |
| 129 | QTPLTMAALELL | CAGACGCCTCTGACTATGGCGGCT |
| | (SEQ ID NO: 88) | CTTGAGCTTCTT |
| | | (SEQ ID NO: 89) |
| 130 | HLQDGSPPSSPH | CAGACGCCTCTGACTATGGCGGCT |
| | (SEQ ID NO: 90) | CTTGAGCTTCTT |
| | | (SEQ ID NO: 91) |
| 131 | GHVTTLSLLSLR | GGGCATGTGACGACTCTTTCTCTT |
| | (SEQ ID NO: 92) | CTGTCGCTGCGG |
| | | (SEQ ID NO: 93) |
| 132 | FPNFDWPLSPWT | TTTCCGAATTTTGATTGGCCTCTG |
| | (SEQ ID NO: 94) | TCTCCGTGGACG |
| | | (SEQ ID NO: 95) |
| 133 | ETPLTEPAFKRH | GAGACGCCTCTTACGGAGCCGGCT |
| | (SEQ ID NO: 96) | TTTAAGCGGCAT |
| | | (SEQ ID NO: 97) |

Analytes. In various embodiments, a target analyte is a marker indicating the existence of a disease, disorder, or condition of the host from which the sample solution was derived.

As used herein the term “Analyte” refers to the compound or composition to be detected or measured and which has at least one epitope or binding site. The analyte can be any substance for which exists a naturally occurring analyte-specific binding member or for which an analyte-specific binding member can be prepared. e.g., carbohydrate and lectin, hormone and receptor, complementary nucleic acids, and the like. Further, possible analytes include virtually any compound, composition, aggregation, or other substance which may be immunologically detected. That is, the analyte, or portion thereof, will be antigenic or haptenic having at least one determinant site, or will be a member of a naturally occurring binding pair.

Analytes include, but are not limited to, toxins, organic compounds, proteins, peptides, microorganisms, bacteria, viruses, amino acids, nucleic acids, carbohydrates, hormones, steroids, vitamins, drugs (including those administered for therapeutic purposes as well as those administered for illicit purposes), pollutants, pesticides, and metabolites of or antibodies to any of the above substances. The term analyte also includes any antigenic substances, haptens, antibodies, macromolecules, and combinations thereof. A non-exhaustive list of exemplary analytes is set forth in U.S. Pat. No. 4,366,241, at column 19, line 7 through column 26, line 42, the disclosure of which is incorporated herein by reference. Further descriptions and listings of representative analytes are found in U.S. Pat. Nos. 4,299,916; 4,275,149; and 4,806,311, all incorporated herein by reference. In some embodiments, the SCD or TD are configured to detect a plurality of different analytes.

Labeled Reagents. The term “labeled reagent” refers to a substance comprising a detectable label attached to a specific binding member (e.g., detection probe). The attachment may be covalent or non-covalent binding, but the method of attachment is not critical. The label allows the label reagent to produce a detectable signal that is related to the presence of analyte in the fluid sample. The specific binding member component of the label reagent is selected to directly bind to the analyte or to indirectly bind the analyte by means of an ancillary specific binding member, which is described in greater detail hereinafter. The label reagent can be incorporated into the TD at a site upstream from the capture zone, it can be combined with the fluid sample to form a fluid solution, it can be added to the test device separately from the test sample, or it can be predeposited or reversibly immobilized at the capture zone. In addition, the specific binding member may be labeled before or during the performance of the assay by means of a suitable attachment method.

“Label” refers to any substance which is capable of producing a signal that is detectable by visual or instrumental means. Various labels suitable for use include labels which produce signals through either chemical or physical means. Such labels can include enzymes and substrates, chromogens, catalysts, fluorescent or fluorescent like compounds and/or particles, magnetic compounds and/or particles, chemiluminescent compounds and or particles, and radioactive labels. Other suitable labels include particulate labels such as colloidal metallic particles such as gold, colloidal non-metallic particles such as selenium or tellurium, dyed or colored particles such as a dyed plastic or a stained microorganism, organic polymer latex particles and liposomes, colored beads, polymer microcapsules, sacs, erythrocytes, erythrocyte ghosts, or other vesicles containing directly visible substances, and the like. Typically, a visually detectable label is used as the label component of the label reagent, thereby providing for the direct visual or instrumental readout of the presence or amount of the analyte in the test sample without the need for additional signal producing components at the detection sites.

Additional labels that can be utilized in the practice of the invention include, chromophores, electrochemical moieties, enzymes, radioactive moieties, phosphorescent groups, fluorescent moieties, chemiluminescent moieties, or quantum dots, or more particularly, radiolabels, fluorophore-labels, quantum dot-labels, chromophore-labels, enzyme-labels, affinity ligand-labels, electromagnetic spin labels, heavy atom labels, probes labeled with nanoparticle light scattering labels or other nanoparticles, fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), epitope tags such as the FLAG or HA epitope, and enzyme tags such as alkaline phosphatase, horseradish peroxidase, I²-galactosidase, alkaline phosphatase, β-galactosidase, or acetylcholinesterase and hapten conjugates such as digoxigenin or dinitrophenyl, or members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc99m, ³⁵S or ³H; or spherical shells, and probes labeled with any other signal generating label known to those of skill in the art. For example, detectable molecules include but are not limited to fluorophores as well as others known in the art as described, for example, in Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland.

A number of signal producing systems may be employed to achieve the objects of the invention. The signal producing system generates a signal that relates to the presence of an analyte (i.e., target molecule) in a sample. The signal producing system may also include all of the reagents required to produce a measurable signal. Other components of the signal producing system may be included in a developer solution and can include substrates, enhancers, activators, chemiluminescent compounds, cofactors, inhibitors, scavengers, metal ions, specific binding substances required for binding of signal generating substances, and the like. Other components of the signal producing system may be coenzymes, substances that react with enzymic products, other enzymes and catalysts, and the like. In some embodiments, the signal producing system provides a signal detectable by external means, by use of electromagnetic radiation, desirably by visual examination. Exemplary signal-producing systems are described in U.S. Pat. No. 5,508,178.

In some embodiments, nucleic acid molecules can be linked to the detection probe (e.g., antibody-linked oligonucleotides), whereby the nucleic acid functions as a label by utilizing nucleic acid labels. For example, a reagent solution or substrate comprised in a SCD can comprise detection reagents comprising a plurality of oligonucleotides functioning to provide a detectable signal, whereby for a Analyte Binding Set (specific for a particular analyte), conjugated oligonucleotides are pre-stained with a different stain as compared to another subpopulation of antibodies (specific for a different analyte) are nucleic acid stains that bind nucleic acid molecules in a sequence independent manner. Examples include intercalating dyes such as phenanthridines and acridines (e.g., ethidium bromide, propidium iodide, hexidium iodide, dihydroethidium, ethidium homodimer-1 and -2, ethidium monoazide, and ACMA); some minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc. Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red). Other detectable markers include chemiluminescent and chromogenic molecules, optical or electron density markers, etc.

As noted above in certain embodiments, labels comprise semiconductor nanocrystals such as quantum dots (i.e., Qdots), described in U.S. Pat. No. 6,207,392. Qdots are commercially available from Quantum Dot Corporation. The semiconductor nanocrystals useful in the practice of the invention include nanocrystals of Group II-VI semiconductors such as MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, and HgTe as well as mixed compositions thereof; as well as nanocrystals of Group III-V semiconductors such as GaAs, InGaAs, InP, and InAs and mixed compositions thereof. The use of Group IV semiconductors such as germanium or silicon, or the use of organic semiconductors, may also be feasible under certain conditions. The semiconductor nanocrystals may also include alloys comprising two or more semiconductors selected from the group consisting of the above Group III-V compounds, Group II-VI compounds, Group IV elements, and combinations of same.

In some embodiments, a fluorescent energy acceptor is linked as a label to a detection probe (i.e., binding moiety conjugated with a detector molecule). In one embodiment the fluorescent energy acceptor may be formed as a result of a compound that reacts with singlet oxygen to form a fluorescent compound or a compound that can react with an auxiliary compound that is thereupon converted to a fluorescent compound. Such auxiliary compounds can be comprised in buffers contained in an SCD and/or TD. In other embodiments, the fluorescent energy acceptor may be incorporated as part of a compound that also includes the chemiluminescer. For example, the fluorescent energy acceptor may include a metal chelate of a rare earth metal such as, e.g., europium, samarium, tellurium and the like. These materials are particularly attractive because of their sharp band of luminescence. In addition, fluorescent lables such as Europium provide at least 2 to 3 logs increased signal over gold particles when detected using a fluorescent reader. Furthermore, lanthanide labels, such as europium (III) provide for effective and prolonged signal emission and are resistant to photo bleaching, thereby allowing TDs containing processed/reacted sample to be set aside if necessary for a prolong period of time.

Long-lifetime fluorescent europium(III) chelate nanoparticles have been shown to be applicable as labels in various heterogeneous and homogeneous immunoassays. See, e.g., Huhtinen et al. Clin. Chem. 2004 October; 50(10): 1935-6. Assay performance can be improved when these intrinsically labeled nanoparticles are used in combination with time-resolved fluorescence detection. In heterogeneous assays, the dynamic range of assays at low concentrations can be extended. Furthermore, the kinetic characteristics of assays can be improved by use of detection antibody-coated high-specific-activity nanoparticle labels instead of conventionally labeled detection antibodies. In homogeneous assays, europium(III) nanoparticles have been shown to be efficient donors in fluorescence resonance energy transfer, enabling simple and rapid high throughput screening. Heterogeneous and homogeneous nanoparticle-label-based assays can be run with various sample matrixes, e.g., serum, heparin plasma, and mucus.

In some embodiments, a label (e.g., fluorescent label) disclosed herein, is comprised as a nanoparticle label conjugated with biomolecules. In other words, a nanoparticle can be utilized with a detection or capture probe. For example, a europium(III)-labeled nanoparticle linked to monoclonal antibodies or streptavidin (SA) to detect a particular analyte in a sample can be utilized (e.g., nanoparticle-based immunoassay). The nanoparticles serve as a substrate to which are attached the specific binding agents to the analyte and either the detection (i.e., label) or capture moiety.

In various embodiments of the invention, the label utilized is a lanthanide metal. Lanthanides include but are not limited to europium, samarium, terbium or dysprosium. Non-specific background fluorescence has a decay time of only about 10 ns, so that such background dies away before the sample fluorescence is measured. Furthermore, Lanthanide-chelates have large Stokes' shifts. For example, the Stokes' shift for europium is almost 300 nm. This big difference between excitation and emission peaks means that the fluorescence measurement is made at a wavelength where the influence of background is minimal. In addition, the emission peak is very sharp which means that the detector can be set to very fine limits and that the emission signals from different lanthanide chelates can be easily distinguished from each other. Therefore, in one embodiment, one or more different lanthanides can be utilized in the same assay.

Hard Standards. In one embodiment, a fluorescence reader is configured to comprise an integrated or permanent standard (“hard standard”). The term “hard standard” as referred to herein means that the device for reading a test sample in methods of detecting/quantifying one or more analytes comprises an internal, integrated or permanent standard, against which samples labeled with the same label as that used in the hard standard are read. In one embodiment, the hard standard and the test label comprise a lanthanide (e.g., Europium III).

In one embodiment, the reader is an LED, comprising a lamp emitting UV A (400 to 315 nm) part of the spectrum. Emission is in the visible part of the spectrum. Some exemplary or conventional LEDs or photodiodes are disclosed in U.S. Pat. Nos. 7,175,086, 7,135,342, and 7,106,442, the disclosure of each of which is incorporated herein in its entirety.

In another embodiment, a reader comprises at least two hard standards of different amounts (e.g., low and high concentration of label), thus providing a two point check of the reader. For example, two (2) lanthanide hard standards (e.g., Europium) are mounted permanently on the reader slides and may be read during the course of each test read. As such, the two hard standards can be utilized to determine the lower detection limit (i.e., in a analyte quantification assay or for determining lowest detection threshold in qualitative assays). Here, fluorescence is read and plotted as percentage of fluorescence (y axis) against concentration (x axis). The straight line between the two reads for each of the hard standards on such a plot allows measuring the intercept of noise (no label) to give a measurement for the lowest detection limit.

In some embodiments, a TD comprises a chamber (compartment or liquid sac) that contains wash or running buffer, which functions to remove unbound label, to reduce or eliminating background noise. In various embodiments, devices comprising a hard standard (s) provide accurate qualitative as well quantitative measurement of analyte(s) present in a sample and labeled with label that is the same as that used in the hard standard(s).

In some embodiments, hard standards are embedded or cast in a polymer material, including glass, plastic, vinyl, or acrylic. Such embedded labels can be cast into appropriate shapes/sizes. Alternatively, such hard standards can be cut to appropriate sizes to be integrated into a reader. In one embodiment, hard standards are cut in rectangular, square, oblong, circular, or any polygon shape. In one embodiment, hard standards are cut into rectangular shapes, comprising dimensions for height of about 0.04, 0.045, 0.05, 0.055, 0.06, 0.065, 0.07, 0.075, 0.08, 0.085, 0.09, 0.095, 0.10, 0.11, 0.12, 0.125, 0.126, 0.127, 0.128, 0.129, 0.130, 0.135, 0.140, 0.150 inch; width of about 0.01, 0.02, 0.03, 0.035, 0.039, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 inch; and lengths of about 0.01, 0.02, 0.03, 0.035, 0.039, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 inch.

In one embodiment, a reader employing a hard standard as a reference is utilized for normalizing readers across a population, e.g., plotting subsequent reader performance against a pre-determined “Gold Standard” reader as illustrated in the following table:

TABLE 4

| | Gold Std. | Test |
|---|---|---|
| S0 | 1000 | 900 |
| S1 | 5400 | 5000 |
| S2 | 10200 | 11000 |
| S3 | 19000 | 20000 |
| S4 | 22000 | 23000 |
| S5 | 50000 | 50000 |

Therefore, where y and x axis are Test reader and Gold Standard measurements respectively, the lower limit of detection is the intercept of the plotted line across the noise level (reading with no label).

In one embodiment, a TD comprises different pRNAs each patterned based on a specific analyte, a complementary SCD comprises a plurality of capture antibody linked to cognate pRNAs to those immobilized on the TD, and where said plurality comprising different subpopulation of antibodies specific for different analytes). Furthermore, the SCD reagent solution or substrate (e.g., lyophilized solid substrate) comprise detection probes, or a plurality of europium(III) labeled antibodies, consisting of the same subpopulations of antibodies specific for different analytes. Additional lanthanide labels are known in the art, such as disclosed in U.S. Pat. No. 7,101,667. See also, e.g., Richardson F. S., “Terbium(III) and Europium(III) Ions as Luminescent probes and Stains for Biomolecular Systems,” Chem. Rev., 82:541-552 (1982).

The reader can report results in timed or read now settings. In timed mode, the reader completes and reports results independent of the operator once the test device has been inserted into the reader. This allows the operator greater freedom to work independently from the machine. The read now mode provides real time results, allowing for batch testing.

pRNA. In one aspect of the invention, combinations of complementary pyranosyl RNA (pRNA) sequences are incorporated in the SCD/Test Devices of the invention as the CMPs allowing simultaneous specific detection of multiple different target analytes. Pyranosyl RNA has been found to have stronger and more selective binding than natural RNA. In addition, pyranosyl-RNA bases stack in a ladder-like fashion, rather than a helical fashion, making stacking interactions favorable and resulting in higher binding affinity. Additionally, pRNA does not interact with endogenous RNA or DNA and is not degraded by RNases, making pRNA ideally suited for use in sample detection. In one embodiment, indoles are used in the pRNA. An indole serves as a neutral base. In various embodiments one of a pair of homologous pRNA sequences is immobilized in a specific stripe or test zone in the TD, while the other of the pair of homologous pRNA sequences is linked to an analyte-specific antibody in the capture probe, thereby allowing binding to a specified target analyte.

In order to minimize cross-reactivity between binding pair pRNA molecules when multiple analytes are studied, binding pair pRNA molecules can be designed to minimize cross-reactivity. An algorithm may be used to determine binding energy between binding partners. For example, the binding programs MFOLD (see http://mfold.bioinfo.rpi.edu/) and BINDIGO (see http://rna.williams.edu/) were created to measure free energy of nucleic acid structures, utilizing the scaling properties of the Smith-Waterman algorithm (Hodas and Aalberts (2004) Nucleic Acids Research 32: 6632-42). Use of algorithms to maximize binding between pRNA CMPs serves to increase both specificity and selectivity. By using this approach, a large number of pRNA sequences can be scanned and sequences having low binding energies for their partner sequences (strong binding) and also have high binding energies for non-partner sequences (weak binding) are selected as ideal pRNA sequences.

In one embodiment, an expert rule based system is used to develop pRNA binding pair in order to minimize cross-reactivity while maintaining high specificity and selectivity binding for pRNA pairs. An expert rule based system utilizes a knowledge base that may have a learning component. In addition, an expert rule based system may utilize information from experimentation or from algorithms such as MFOLD and BINDIGO, as described above. In one embodiment, resulting pRNA pairs have been identified which have high affinity for each other with little to no affinity for non-homologous pairs.

In some embodiments, pRNA CMPs are selected from but not limited to the pRNAs shown in Table 5.

TABLE 5

| Name | 4′-2′ | SEQ ID NO: |
|---|---|---|
| 102a10-3-NH2 | TAGAACGAAG | 98 |
| 102b10-3-NH2 | CTTCGTTCTA | 99 |
| 119a10-1-NH2 | TCAGTGGATG | 100 |
| 119b10-1-NH2 | CATCCACTGA | 101 |
| 3a10-1-NH2 | GTATTGCGAG | 102 |
| 3b10-1-NH2 | CTCGCAATAC | 103 |
| 102a8-2-NH2 | AACGATTC | 104 |
| 102b8-2-NH2 | GAATCGTT | 105 |
| 119a8-1-NH2 | AGTGGATG | 106 |
| 119b8-1-NH2 | CATCCACT | 107 |
| 3a8-1-NH2 | GTATTGCG | 108 |
| 3b8-1-NH2 | CGCAATAC | 109 |
| 4a8 | ATGCCTTC | 110 |
| 4b8 | GAAGGCAT | 111 |
| 5a8 | TGATGGAC | 112 |
| 5b8 | GTCCATCA | 113 |
| 6a8 | CAGTAGTG | 114 |
| 6b8 | CACTACTG | 115 |
| 7a8 | TTCCTGAG | 116 |
| 7b8 | CTCAGGAA | 117 |
| 8a8 | GACTCTCT | 118 |
| 8b8 | AGAGAGTC | 119 |
| 4a9-In | ATGCDCTTC | 120 |
| 4b8-In | GAADGCAT | 121 |
| 5b9-In | GTCDCATCA | 122 |
| 6a6 | CAGTAG | 123 |
| 6b6 | CTACTG | 124 |
| 8a6 | GACTCT | 125 |
| 8b6 | AGAGTC | 126 |

all oligos with 4′-C12 amino and 2′-hexanol groups

In one embodiment, pRNA pairs are selected to minimize cross reactivity with other pRNA when multiple pRNA sequences are used to detect multiple analytes. Minimization of cross-reactivity allows for generation of a cleaner signal and reduces artificial binding that can create false positive results. Certain pRNA sequences in Table were selected in order to maximize binding between pRNA partners while minimizing binding to other binding pairs. For example, the pRNA sequences of SEQ ID NOs: 120-126 were designed to minimize cross reactive binding to each other. pRNAs that have been specifically selected to minimize cross-reactivity (e.g., SEQ ID NOs: 120-126) will have decreased cross-reactivity to other pRNA binding pairs by at least 5%, 10%, 15%, 20%, 25%, 30%, 35%, 40%, 50%, 60%, 70%, 80%, 90% or greater. Assays for determining cross-reactivity are known in the art and include, for example, a competition assay or ELISA. In another embodiment, pRNA CMPs that have been specifically selected to minimize cross-reactivity (e.g., SEQ ID NOs: 120-126) will have decreased cross-reactivity to other pRNA by an EC50 concentration that is 1 nM, 5 nM, 10 nM, 20 nM, 30 nM, 50 nM, 100 nM, 250 nM, 500 nM, 1 μM or greater. In another embodiment, pRNA CMPs that have been specifically selected to minimize cross-reactivity (e.g., SEQ ID NOs: 120-126) will have decreased cross-reactivity to other pRNA by an EC50 concentration that is 1×, 2×, 3×, 4×, 5×, 6×, 7×, 8×, 9×, 10× or greater fold decrease compared to binding of non-partner sequences. In various embodiments, pRNAs are utilized as CMPs and ICMPs.

In various embodiments, a pRNA molecule that is immobilized on a test strip at an addressable line will bind specifically to the complimentary pRNA conjugated with anti-analyte binding agents (e.g., anti-virus antibody).

In some embodiments, a TD incorporating one or more immobilized pRNA, is capable of providing sensitivity of about 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.2, 1.5, 1.7, 2.0, 2.5, 3.0, 3.5, 4.0, 4.5, 5.0, 5.5, 6.0, 6.5, 7.0, 7.5, 8.0, 8.5, 9.0, 9.5, 10.0, 15, 20, 30, 40 or 50 ng/mL for detection of a target analyte. The term “about” in this context refers to +1-5% of a given measurement.

In some embodiments, a TD incorporating one or more immobilized pRNA, is capable of providing sensitivity of at least 70%, 80%, 90%, 91%, 92%, 93%, 94%, 95%, 96%, 97%, 98%, or 99% against a control assay, such as a growth culture or real-time PCR test, as described in Example 1. Sensitivity is meant to describe the positive rate generated by the test assay.

In some embodiments, a TD incorporating one or more immobilized pRNA, is capable of providing specificity of about 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.2, 1.5, 1.7, 2.0, 2.5, 3.0, 3.5, 4.0, 4.5, 5.0, 5.5, 6.0, 6.5, 7.0, 7.5, 8.0, 8.5, 9.0, 9.5, 10.0, 15, 20, 30, 40 or 50 ng/mL for detection of a target analyte.

In some embodiments, a TD incorporating one or more immobilized pRNA, is capable of providing specificity of at least 70%, 80%, 90%, 91%, 92%, 93%, 94%, 95%, 96%, 97%, 98%, or 99% against a control assay, such as a growth culture or real-time PCR test, as described in Example 1. Specificity is meant to describe the negative rate generated by the test assay.

In some embodiments pRNA is attached to a membrane (i.e., test strip) utilizing a linker, for example, a protein linker. For example, pRNA can be conjugated to a hydrophilic protein. In one embodiment, the linker protein has a molecular weight of at least from about 500, 600, 700, 800, 900, 1000, 1500, 2000, 2500, 3000, 3500, 4000, 4500, 5000, 5500, 6000, 6500, 7500, 8000, 9000, 10000, 20000, 30000, 40000, 50000, 60000, 70000, 80000, 90000, 100000, 110000, 120000, 130000, 140000, 150000, 160000, 170000, 180000, 190000, 200000, 225000, 250000, 300000, 350000 to about 450000. Such a linker can range in size from about 5 to 10, 6 to 11, 7 to 12, 8 to 13, 9 to 14, 10 to 15, 11 to 16, 12 to 17, 13 to 18, 14 to 19, 15 to 20, 16 to 21, 17 to 22, 18 to 23, 19 to 24, 20 to 25, 21 to 26, 22 to 27, 23 to 28, 24 to 29, 25 to 30, 35, 40, 45 or 50 AA long. The linker can be a peptide or polypeptide. In one embodiment, the linker is BSA or IgG.

In another embodiment the linker is a monoclonal antibody. The linker can serve as an anchor protein for binding the pRNA to the test device. Anchor protein conjugates may be purified using standard methods known in the art, for example, by purification over a Sephacryl-300 column. In one embodiment, the anchor protein is the linker IgG MAb 2-199-C (Abcam, Cambridge, Mass.), a monoclonal antibody specific for rodent Cytochrome-C. MAb 2-199-C conjugated pRNA results in an increased signal-to-noise ratio compared to pRNA alone. In another embodiment, the anchor protein is bovine serum albumin (BSA). In a specific embodiment, the BSA used is single chain BSA. Use of an anchor protein and/or spacer arm allows striping a greater concentration of an ICMP, therefore enhancing the sensitivity and/or specificity of an assay of the invention.

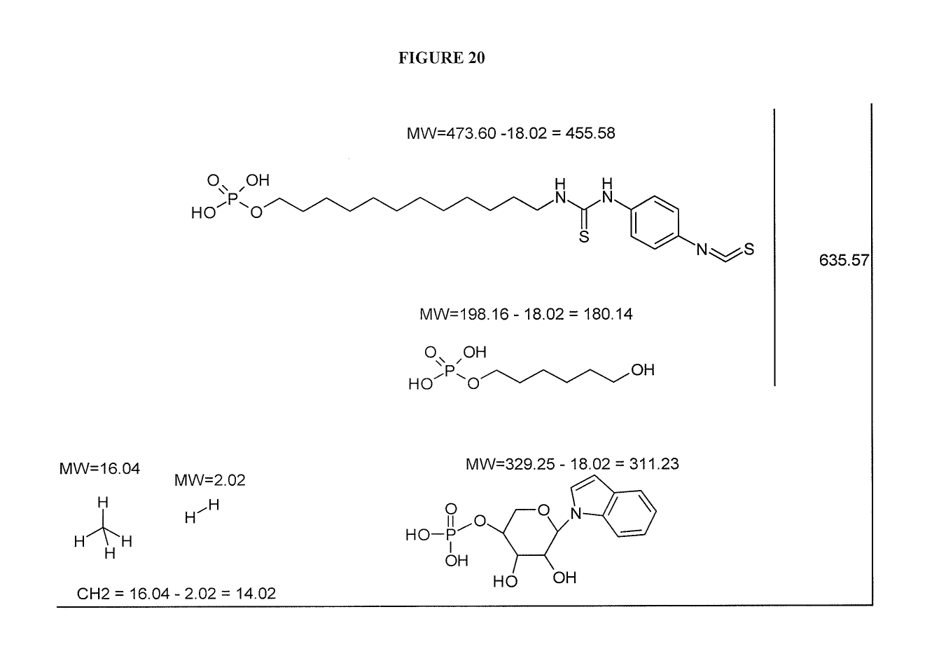

In yet another embodiment, an antibody can be attached to a pRNA molecule via a separate linker, such as a carbon spacer. In one embodiment, the carbon spacer has a phosphate group at one end. The carbon spacer can have any number of carbon atoms, for example, 2, 3, 4, 5, 6, 7, 8, 9, 10, 11, 12, 13, 14, 15, 20, 25, 30 or more carbon atoms in the carbon spacer. Examples of linker molecules are shown in FIG. 20 (top structure). The phosphate group can attach to a nucleotide, for example, at the 4′ end of the nucleotide. In some instances, the activation chemistry is based on modification of the amino group with 1,4-phenylene diisothiocyanate (PDITC). PDITC is a homobifunctional cross-linker containing two amine-reactive isothiocyanate groups on a phenyl ring. Reaction in excess with amine-modified pRNA oligomer results in the formation of a thiourea linkage, leaving the second isothiocyanate group free to couple with amine-containing molecules such as proteins. The PDITC chemistry is advantageous because it is sufficiently stable at neutral pH and in dry state so it can be purified by reverse-phase HPLC and stored without significant decomposition for months. Also, when contacted with proteins at slightly basic pH, PDITC-activated pRNA efficiently and selectively reacts with primary amino groups of lysines affording a stable pRNA-protein linkage. For example, FIG. 20 shows a structure of PDITC linked to a 12-carbon spacer. The PDITC-linker-phosphate can be added to a binding partner, such as an oligonucleotide or pRNA molecule, via the phosphate instead of a new nucleotide at the 4′ end of an oligonucleotide or pRNA molecule. In some embodiments, the pRNA is kept at an acidic pH, for example below pH 5, 4, 3, or 2 prior to conjugating with a linker or protein. For example, the pRNA can be kept stable at pH 2.2 prior to conjugation. The pH of the pRNA can be raised prior to a conjugation reaction. For example, the pH of the pRNA can be brought to pH 8.5 prior to a conjugation reaction. In other embodiments, the pRNA can be stored dried prior to conjugation with a linker or protein.

In one embodiment, a TD comprises ICMPs that are bound to the test strip by an anchor protein. In one embodiment, the ICMP bound to anchor protein is a pRNA.

In one embodiment, pRNA is coupled to a hydrophilic protein/peptide via a covalent bond between the pRNA molecule and the hydrophilic protein. A solution containing the pRNA-protein complex is applied to defined regions on a test membrane (e.g., nitrocellulose), whereby the protein anchor binds to the membrane in an irreversible manner. The pRNA is then available for use in the assay. In one embodiment, the anchor/linker protein is a hydrophilic protein and the test membrane is nitrocellulose.

In another embodiment, pRNA is conjugated via a linker to an immobilizing molecule. The linker may be a carbon linker and may have 1, 2, 3, 4, 5, 6, 7, 8, 9, 10, 11, 12, 13, 14, 15 or more carbons in the linker. The immobilizing molecule may be, for example, a diisothiocyanate, such as 1,4-phenylene diisothiocyanate. Oligomers that are conjugated to an immobilizing molecule are subject to post-synthesis purification. For example, the oligomers may be purified over a gel filtration column to separate products by size, such as a Sephacryl-300 column (GE Healthcare Life Sciences, Pittsburgh, Pa.). Additionally, the oligomers may be analyzed for reagent purity. For example, matrix-assisted laser desorption/ionization, time-of-flight mass spectrometry may be used to determine the identity and purity of the conjugated oligonucleotide product. The proportion of pRNA molecules to anchor proteins and/or antibodies (collectively referred to as “CMP binding proteins”) can vary in a mixture to produce pRNA-CMP binding protein conjugates, as will their concentrations in the reaction mixture. In general, the higher the specific activity of pRNA-CMP binding protein conjugates (moles pRNA per mole CMP binding protein) the better the assay performance. The optimal ratio of pRNA to CMP binding protein can be determined for each pRNA+CMP binding protein combination. Above a certain ratio, the addition of additional pRNA to the CMP binding protein can begin to generate high molecular weight (HMW) aggregates not observed in the CMP binding protein starting material. These HMW aggregates can be seen by size exclusion chromatography (SEC). Without being bound by theory, the formation of HMW aggregates is most likely due to non-specific electrostatic interactions and not due to protein-protein cross linking during the conjugation reaction as the pRNA contains only a single reactive moiety per pRNA oligomer as confirmed by quality control testing analysis. In support of this theory, no protein-protein cross linking is observed when pRNA-CMP binding protein conjugates are chromatographed by denaturing SDS capillary electrophoresis. The observed mobility shifts of conjugated CMP binding proteins correspond to the addition of 1, 2, 3 or 4 pRNA molecules per CMP binding protein and higher levels of pRNA incorporation were not resolved into discreetly resolved species. However, the shift in conjugate size does not correspond to covalent protein-protein dimers and trimers. The presence of the contaminating HMW material generated is in direct proportion to pRNA specific activity of the pRNA-CMP binding protein conjugate (moles pRNA per mole CMP binding protein) which reflects the ratio of reactants in the conjugation reaction. The HMW aggregates can produce non-specific binding to pRNA test lines striped onto nitrocellulose. In some embodiments, removal of the HMW material can be performed to maintain specific pRNA/pRNA interactions in the assay. Various techniques known in the art, including size exclusion chromatography (SEC) can be used to remove the HMW aggregates from the monomeric pRNA-CMP binding protein conjugate. SEC removal of HMW aggregates provides a mechanism for increasing assay sensitivity by increasing the pRNA specific activity of pRNA-CMP binding protein conjugates without introducing material which produces non-specific binding to other pRNA test lines. As an example, an SEC separation of HMW material from an antibody-pRNA conjugate can be performed and shown in a Sephacryl 300 HR chromatograph. The HMW material elutes first from the column (minutes 87-108) followed by the antibody-pRNA conjugate (minutes 108-125). Two other peaks of material elute at minutes 165-195 and represent unincorporated pRNA. Production of high specific activity pRNA conjugates can be improved through the removal of the HMW fraction in order to maintain good assay performance with respect to binding specificity and sensitivity. The limit as to how high the pRNA specific activity can be increased is set by the respective yield of monomeric un-aggregated pRNA-CMP binding protein conjugate that can be obtained from the SEC chromatography.

In some embodiments, the pRNA has a specific activity of at least 20%, 30%, 40%, 50%, 60%, 70%, 80%, 90%, or 100% of that of the pRNA that did not have removal of the HMW fraction. Methods of assaying and quantifying measures of enzymatic activity and substrate specificity, are well known to those of skill in the art.

In another embodiment, (e.g, FIG. 18) a TD 1807 comprises a plurality of addressable test lines utilizing different categories of CMPs (e.g., a combination of antibodies, nucleic acids, pRNA, avidin/streptavidin/biotin).

In one embodiment shown in FIG. 18, at least one addressable line or specific capture zone 1805, 1812 comprises a pRNA ICMP 1804, 1811 having pRNA 1803 that is bound to a solid support 1808 (e.g., nitrocellulose, polystyrene, glass, plastic, metal, etc.) and is specific in binding to a cognate or complementary pRNA sequence conjugated 1807 to an antibody to form a capture probe 1802, 1810 that is specific for a particular target analyte 1806. A detection probe 1801 contains an antibody also capable of specifically binding the analyte conjugated to a label 1809

An immune complex formed of a detection probe-target analyte-capture probe can be effectively immobilized and can specifically bind to the addressable line comprising the ICMP that is specific for the CMP comprised in said capture probe (e.g., complementary or cognate pRNA pairs).

In one embodiment, pRNA molecules are comprised in a capture probe as a CMP and for each pRNA used there in a capture probe there is disposed on one addressable lines a complementary immobilized pRNA (i.e., ICMP). In one embodiment, a test strip comprises a plurality of addressable lines comprising pRNAs, such as on 1, 2, 3, 4, 5, 6, or 7 distinct addressable lines on a test strip.

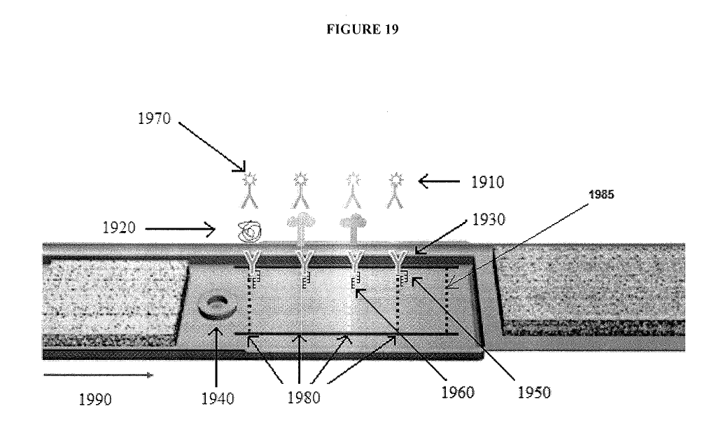

In one embodiment, a TD comprises a test strip having a plurality of test zones, wherein each test zone is specific for a distinct analyte (e.g., influenza type A or B) and/or subtype (e.g., influenza A pandemic and non-pandemic subtypes). In one embodiment (e.g., FIG. 19) a TD comprises a test strip with at least four test zones, wherein one test zone is configured for detection of influenza A virus or a component thereof, a second test zone is configured for detection of a subtype of influenza A, e.g., H1, a third test zone that is configured for detection of a second subtype of influenza A, e.g., H3 and a fourth test zone configured for detection of influenza B. In a further embodiment, each test zone comprises a different ICMPs, such that each comprises a pRNA sequence selected from the group consisting of SEQ ID NO: 120 to SEQ ID NO: 126.

A test device may utilize a variety of species or categories of capture moieties (e.g., pRNA and avidin/streptavidin) in combination. Thus, for example, two test zones can utilize pRNA as a partner capture moiety, while other test zones utilize strepatvidin/avidin-biotin, a fixed antibody, or DNA/RNA. For clarity, in the context of a capture probe and an ICMP disposed on one test zone, the CMP and ICMP are selected and utilized in the various embodiments of the invention based on their specific binding for each other (e.g., a pRNA binding to it complementary pRNA, an antibody binding to its target antigen, avidin binding to biotin, etc.).

In order to further minimize cross-reactivity between ICMPs and/or CMPs, addressable lines may be configured such that a ICMP of one type or category is not next to an adjacent addressable line having the same category of ICMP. For example, an antibody ICMPs is placed on addressable lines 1, 3 and 5, but different ICMPs (e.g. pRNA or avidin/streptavidin/biotin) is placed on addressable lines 2 and 4.

In another embodiment, the same type of ICMP may be used, e.g., all test zones comprise pRNAs, but pRNAs on any two adjacent lines are selected based on displaying reduced cross-reactivity. In one embodiment, each of one, two, three or four test zones comprises a different pRNA sequence, with at least one pRNA selected from SEQ ID NO: 120 to SEQ ID NO: 126.

In one embodiment, pRNA are spaced such that there is a spacer line separating each addressable line comprising a pRNA, such that a pRNA addressable line is not immediately adjacent to another pRNA addressable line.

In yet another embodiment, a combination of different types of capture moiety partners are used (e.g., a combination of antibodies, nucleic acids, pRNA, avidin/streptavidin/biotin) on different multiple addressable test/capture zones such that a particular category of partner capture moiety is not located on an addressable test/capture zone that is adjacent to the same category of partner capture moiety. For example, if an antibody partner capture moiety were used in addressable test/capture zone 2, then addressable test/capture zones 1 and 3 would not contain an antibody partner capture moiety, but could instead have a pRNA, nucleic acid, avidin/streptavidin/biotin partner capture moiety or a control or blank line. By interspacing each category of partner capture moiety with a different category of partner capture moiety, it is possible to decrease the amount of cross reactivity that is present between addressable test/capture zones.

In one embodiment, interspacing each category of partner capture moiety with a different category of partner capture moiety decreases the amount of cross reactivity by at least 5%, 10%, 15%, 20%, 25%, 30%, 35%, 40%, 50%, 60%, 70%, 80%, 90%, 95%, 96%, 97%, 98%, 99% or greater thus providing a more specific assay. A test device having interspaced types of capture moiety partner can be measured for decreased cross-reactivity by comparing binding to a similar device not having interspaced types of partner capture moieties (e.g. antibody partner capture moieties are placed on adjacent addressable test/capture zones).

Various concentrations of pRNA can be bound to an addressable line. In some embodiments, the concentration of pRNA on a test line can be from 1.0 pg/mm of strip width to 1000 ng/mm of strip width, or 2.0 pg/mm of strip width to 500 ng/mm of strip width, or 2.5 pg/mm of strip width to 200 ng/mm of strip width. In some embodiments, for a test strip that is about 5 mm wide, the concentration of pRNA bound to the test strip is from 10 ng/strip to 10000 ng/strip, or 20 ng/strip to 5000 ng/strip, or 30 ng/strip to 4000 ng/strip.

As such, a central aspect of the present SCD/TDs of the invention is that they can be configured to detect multiple analytes including, but not limited to cells, cell components (e.g., cell markers, cell surface markers), and proteins (e.g., enzymes).

In one embodiment, SCD/TDs of the invention are used in a method to assay for any pathogenic conditions for which particular corresponding analytes are known or are identified in future. The SCD and TD can be configured to provide any combination of the capture probes and detection probes disclosed herein. For example, multiple analytes corresponding to myocardial infarction (MI) can be identified in detecting/diagnosing MI. Markers for various conditions are known in the art, such as for cardiac markers disclosed in U.S. Pat. Nos. 5,604,105; 5,710,008; 5,747,274, 5,744,358 and 5,290,678, the disclosures of each of which is incorporated by reference herein in its entirety.

In one embodiment, a mixture of sample and SCD buffers and/or reagents is formed in an SCD and flows from the SCD and through the TD via any of several mechanisms, including capillary action, hydrostatic pressure, or other non-capillary action along the surface of or within a matrix of a solid material/substrate (e.g., test strip). If a target analyte is present, a complex is formed comprising a capture probe-analyte-detection probe and such a complex when run through a test strip will accumulate at a specified test zone yielding a signal that can be interpreted by the naked eye or using an instrument reader.

Aptamers. In some embodiments, aptamers are used as either capture moiety partners or analyte-specific binding agents, or both in SCDs and TDs of the invention. Aptamers include nucleic acids that are identified from a candidate mixture of nucleic acids. In one embodiment, an aptamer is used to bind a target analyte, and thus the analyte is the analyte-specific binding agent in a capture probe, detection probe or both the capture probe or detection probe.

In various embodiments, aptamers include nucleic acid sequences that are substantially homologous to the nucleic acid ligands isolated by the SELEX method, based on binding specificity to a target analyte (e.g., infectious agents disclosed herein). Substantially homologous is meant a degree of primary sequence homology in excess of 70%, most preferably in excess of 80%. The “SELEX” methodology, as used herein, involves the combination of selected nucleic acid ligands, which interact with a target analyte in a desired action, for example binding to a protein, with amplification of those selected nucleic acids. Optional iterative cycling of the selection/amplification steps allows selection of one or a small number of nucleic acids, which interact most strongly with the target antigen/biomarker from a pool, which contains a very large number of nucleic acids. Cycling of the selection/amplification procedure is continued until a selected goal is achieved. The SELEX methodology is described in the following U.S. patents and patent applications: U.S. patent application Ser. No. 07/536,428 and U.S. Pat. Nos. 5,475,096 and 5,270,163.

Infectious Agents. In various embodiments of the present compositions and methods, an infectious agent can be any pathogen including without any limitation bacteria, yeast, fungi, virus, eukaryotic parasites, etc. In some embodiments, the infectious agent is influenza virus, parainfluenza virus, adenovirus, rhinovirus, coronavirus, hepatitis viruses A, B, C, D, E, etc, HIV, enterovirus, papillomavirus, coxsackievirus, herpes simplex virus, or Epstein-Barr virus. In other embodiments, the infectious agent is Mycobacterium, Streptococcus, Salmonella, Shigella, Staphylcococcus, Neisseria, Clostridium, or E. coli. It will be apparent to one of skill in the art that the compositions and methods of the invention are readily adaptable to different infectious agents, by utilizing a different panel of binding agents (e.g., antibodies) that are specific for type(s) or subtype(s) of an infectious agent(s).

Usually the general type of an infectious agent can be the genus type of an infectious agent or any primary or first instance typing or identification of an infectious agent. A subtype of an infectious agent can be the species or strain type of an infectious agent or any secondary or subsequent typing of an infectious agent. Identification of the general type or subtype of an infectious agent can be carried out via various suitable test set ups. For example, identification of the general type of an infectious agent can include one or more screening tests for 1) a specific general type of an infectious agent, 2) certain desired or selected general types of an infectious agent, or 3) all or substantially all relevant general types of an infectious agent, or a combination thereof. Similarly identification of the subtype of an infectious agent can include one or more screening tests for 1) one or more specific subtypes of an infectious agent, 2) one or more specific subtypes of a particular general type of an infectious agent, 3) one or more specific subtypes of an infectious agent selected based on additional information associated with the subject being tested, e.g., one or more suspected or expected subtypes for a particular population or geographic location or 4) one or more potentially pandemic or epidemic subtypes of an infectious agent that is identical to or associated with the infectious agent tested for the general type, or a combination thereof.

According to another aspect, the method can optionally or additionally include identification of the general and/or subtype(s) of a second infectious agent that is closely related to the first infectious agent, or alternatively the infection of the second infectious agent is associated or likely coupled with the infection of the first infectious agent. For example, HIV infection can be associated with certain bacterial infections therefore it will be useful to identify the general and subtype(s) of HIV as well as Mycobacterium and/or Pneumocystis carinii. Therefore, in one embodiment, the method includes identification of the general and subtype(s) of a virus as well as a bacterium. In another embodiment, the method provided by the various embodiments of the invention includes identification of the general and subtype(s) of a first virus as well as a second virus. For example, a method is provided for identification of the general and subtype(s) of HIV as well as hepatitis virus. Another example would be in testing patients for influenza infection, where mutation or variation of the strains within subtypes is known to occur and some forms of influenza are far more pathogenic than others. A further example is detection of different types of HIV, for example HIV-1 and HIV-2. In one aspect, identification of the general type of human immunodeficiency virus (HIV) can include screening for the presence of HIV whereas identification of the subtype of HIV can include screening for HIV-1, HIV-2, and/or other subtypes of HIV. Similarly identification of the general type of herpes virus such as simplex virus (HSV) can include screening for the presence of HSV whereas identification of the subtype of HSV can include screening for HSV type 1 and/or HSV type 2 or for Epstein-Barr virus (EBV) and subtypes of EBV.

In still another particular aspect, identification of the general type of enterovirus can include screening for the presence of one or more enteroviruses, e.g., poliovirus, coxsackievirus, echovirus, designated enterovirus, etc. whereas identification of the subtype of enterovirus can include screening for poliovirus, e.g., serotype 1-3, coxsackievirus A, e.g., serotype 1-22 and 24, coxsackievirus B, e.g., serotype 1-6, echovirus, e.g., serotype 1-9, 11-27, 29-31, and designated enterovirus, e.g., enterovirus 68-71, etc.

In one embodiment, the methods and apparatus of the invention are utilized to detect or identify an influenza type A subtype and/or influenza type B and/or influenza type C. Influenza virus belongs to the genus orthomyxovirus in the family of Orthomyxoviridae. ssRNA enveloped viruses with a helical symmetry. Enveloped particles 80-120 nm in diameter. The RNA is closely associated with the nucleoprotein (NP) to form a helical structure. The genome is segmented, with 8 RNA fragments (7 for influenza C). There are 4 principle antigens present, the hemagglutinin (H), neuraminidase (N), nucleoprotein (NP), and the matrix (M) proteins. The NP is a type-specific antigen which occurs in 3 forms, A, B and C, which provides the basis for the classification of human and non-human influenza viruses. The matrix protein (M protein) surrounds the nucleocapsid and makes up 35-45% of the particle mass. Furthermore, 2 surface glycoproteins are seen on the surface as rod-shaped projections. The haemagglutinin (H) is made up of 2 subunits, H1 and H2. Haemagglutinin mediates the attachment of the virus to the cellular receptor. Neuraminidase molecules are present in lesser quantities in the envelope. The antigenic differences of the hemagglutinin and the neuraminidase antigens of influenza A viruses provide the basis of their classification into subtypes. e.g., A/Hong Kong/I/68 (H3N2) signifies an influenza A virus isolated from a patient in 1968, and of subtype H3N2.

In various embodiments, the methods and apparatus of the invention are directed to detecting or identifying influenza virus type A which is defined by HxNy where x is 1-16 and y is 1-9, or any combination of xy thereof. For example, in one embodiment, the methods and apparatus of the invention is utilized to detect influenza A subtype H1N5. Thus, a plurality of detection probes and capture probes targeting different subtypes of influenza virus are disposed in an SCD of the invention. In several embodiments, the assay can utilize various combinations of detection probes to detect Influenza A (with subtypes H1/H3, and a pandemic subtype H5) and Influenza B.

In particular, the general type of an influenza virus can be any type designated based on antigenic characteristics of the nucleoprotein and matrix protein antigens, e.g., type A, B, or C influenza virus, whereas the subtype can be one or more subdivided types of an influenza virus on the basis of an antigen, e.g. one or more subtypes of influenza type A or type B virus characterized by a surface antigen such as hemagglutinin (H) or neuraminidase (N).

In one embodiment, identification of the general type of influenza virus includes screening for type A, type B, and/or type C influenza virus whereas identification of the subtype of influenza virus, e.g., type A virus includes screening for one or more expected subtypes of type A, e.g., subtypes expected to be present in the population at the time of testing, and optionally one or more suspected subtypes, e.g., subtypes under surveillance for an outbreak such as epidemic or pandemic outbreak. In another embodiment, identification of the general type of influenza virus includes screening for type A and type B influenza virus whereas identification of the subtype of influenza virus, e.g., type A virus includes screening for one or more subtypes used for the production of the influenza vaccine, e.g., current vaccine subtypes(s) or strain(s) for the testing season including subtypes and/or strains expected to be in circulation during the next influenza season. In yet another embodiment, identification of the general type of influenza virus includes screening for type A and type B influenza virus whereas identification of the subtype of influenza virus, e.g., type A includes screening for one or more subtype(s) or strain(s) used for the production of the influenza vaccine and one or more subtype(s) or strain(s) suspected for the cause of a pandemic outbreak, e.g., one or more avian subtype(s) or strain(s) such as H5N1 or the derivatives thereof or one or more swine subtype(s) or strain(s) such as H1N1.

In yet another embodiment, identification of general type of influenza virus includes screening for type A and type B influenza virus whereas identification of the subtype of an influenza virus, e.g., type A includes screening for one or more common or expected subtypes in circulation including, without any limitation, a) H₁ and H₃, b) H₁, H₃, and H₂, c) H₁, H₂, H₃, and H₉, d) H₁, H₃, and N₁, e) H₁, H₃, N₁, and N₂, f) H₁, H₃, and N₂ g) N₂, and h) N₁ and N₂. For example, a screening test for the subtype identification of type A influenza virus can be directed to the identification of the presence of any one of the subtypes listed in the subtype group of a), b), c), d), e), f), g), or h) e.g., without necessarily identifying the presence of a specific subtype in a subtype group. Alternatively screening test for the subtype identification of type A influenza virus can be directed to the identification of the presence or absence of each and everyone of the subtypes listed in a), b), c), d), e), g), or h) e.g., identifying the presence of a specific subtype in a subtype group.

In still another embodiment, identification of general type of influenza virus includes screening for type A and type B influenza virus whereas identification of the subtype of an influenza virus, e.g., type A includes screening for one or more pandemic or un-expected subtypes in circulation including, without any limitation, a) H₅, b) H₅ and H₇, c) H₅, H₇, and H₉, d) N₂, N₇, and N_{g}, e) H₅ and N₂, f) H₅ and N₁, g) H₅ and N_{g}, h) H₅, N₈, H₇, and N₇, i) H₅, H₇, H₉, N₇, and N₈. For example, a screening test for the subtype identification of type A influenza virus can be directed to the identification of the presence of any one of the subtypes listed in the subtype group of a), b), c), d), e), f), g), h), or i) e.g., without necessarily identifying the presence of a specific subtype in a subtype group. Alternatively screening test for the subtype identification of type A influenza virus can be directed to the identification of the presence or absence of each and everyone of the subtypes listed in a), b), c), d), e), f), g), h), or i), e.g., identifying the presence of a specific subtype in a subtype group.

In another particular aspect, the general type of hepatitis virus can be A, B, and C virus with each virus possibly having several subtypes including mutant strains. In one embodiment, identification of the general type of hepatitis virus includes screening for A, B, and/or C hepatitis virus whereas identification of the subtype of hepatitis virus includes screening for subtypes or mutant strains of A, B, and C hepatitis viruses, respectively. In another embodiment, identification of the general type of hepatitis virus includes screening for hepatitis B virus whereas identification of the subtype of hepatitis virus includes screening for one or more subtypes and/or mutant strains of hepatitis B virus. In yet another embodiment, identification of the general type of hepatitis virus includes screening for hepatitis C virus whereas identification of the subtype of hepatitis virus includes screening for one or more of subtypes 1-9 of type C hepatitis virus.

In general, with respect to a bacterial infectious agent identification of the general and subtype of a bacterial infectious agent includes screening for the genus and one or more species or strains of the bacterial infectious agent that are relevant to the infection and/or the agent's antimicrobial resistance. In one embodiment, identification of the general and subtype of a bacterial infectious agent includes screening for Mycobacterium and one or more species of Mycobacterium including without limitation M. tuberculosis, M. avium, M. bovis, M. chelonei, M. fortuitum, M. intracellulare, M. kansasii, M. leprae, etc. In another embodiment, identification of the general and subtype of a bacterial infectious agent includes screening for Salmonella and one or more species of Salmonella including without limitation S. typhi, S. enteritidis, etc. In yet another embodiment, identification of the general and subtype of a bacterial infectious agent includes screening for Shigella and one or more species of Shigella including without limitation Sh. dysenteriae. In yet another embodiment, identification of the general and subtype of a bacterial infectious agent includes screening for Streptococcus and one or more species of Streptococcus including without limitation S. pneumonia, S. pyogenes (group A), etc. In still yet another embodiment, identification of the general and subtype of a bacterial infectious agent includes screening for E. coli and one or more strains of E. coli including without limitation enterotoxigenic strains.

According to various embodiments of the invention, screening test(s) used for the identification of the general and subtype(s) of an infectious agent can be any suitable tests known or later discovered in the field. For example, the screening tests can be a non-nucleic acid based test including without any limitation a protein, peptide, amino acid, ligand, or chemistry based test. In one embodiment, a method is provided for detection based on the presence or absence of one or more structural proteins of an infectious agent, e.g., glycoproteins, envelop proteins, polysaccharides, etc. In another embodiment, a test is based on the presence or absence of one or more antigens or epitopes, or antibodies to an infectious agent. In yet another embodiment, a test is based on the presence or absence of one or more substances that is released or metabolized by an infectious agent. In still yet another embodiment, a test is based on the presence or absence of one or more substances derived from a host cell associated with or generated by the infection of an infectious agent.

In various embodiments, methods and apparatuses of the invention can detect one or more different infectious agents. For example, a sampling implement can comprise a plurality of different antibodies, wherein multiple subgroups of antibodies are present, whereby each subgroup of antibodies specifically binds a different infectious agent. For example, a plurality of antibodies can comprise 2, 3, 4, 5, 6, 7, 8, 9 or 10 subgroups, wherein each subgroup of antibodies in the plurality of antibodies specifically binds a different infectious agent. In some embodiments, methods and apparatus of the invention detect a pandemic and non-pandemic infectious agent. In one embodiment, the pandemic and non-pandemic infectious agents are influenza virus. In some circumstances such sample collection and processing will necessarily occur in a point-of-care setting (e.g., in the field, without large numbers of subjects to sample and process, and with limited man power to effect such sampling).

As such, in one embodiment, the methods and apparatus of the invention are utilized in processing a large number of samples, in a point-of-care setting, where test results may be visualized (i.e., read) some period of time after the test is complete. For example, the period of time can be 30 minutes, 1 hour, 1.5 hour, 2 hours, 2.5 hours, 3 hours, 4 hours or 5 hours. In some embodiments, methods and apparatus in conjunction with the reagents disclosed herein provide high sensitivity and specificity where the fluorescent result can be read with very similar results over a long period of time. Thus, in some embodiments biological samples can be collected and processed, but set aside to be read a significant time later, which is greatly advantageous in point-of-care settings or where a large number of samples are collected with limited manpower or time to further process samples.

In yet another aspect of the invention, the compositions and methods of the invention are directed to detecting any one or more analytes present in a sample. As indicated above, for example, by utilizing different binding agents that specifically bind markers associated with a condition, one or more analytes associated with MI can be detected. Therefore, an SCD and TD can comprise the necessary reagents to diagnose a disease or pathological condition, other than infectious diseases.

In some embodiments, the one or more analytes are markers associated with a pathological condition or disease. In another embodiment, the one or more analytes are polypeptides associated with a nutrional state or condition. In yet other embodiments, the one or more analytes are cell markers associated with cell cycle and growth. In another embodiment, the one or more analytes are associated with cell proliferation and differentiation. In one embodiment, cell markers are associated with cancer.

### EXAMPLES

### Example 1

### Detection of Influenza During 2007 Australian Flu Season

A set of 121 nasopharyngeal swab samples were collected during 2007 Australian flu season. After the nasal samples were collected, the swabs were placed in 1 mL of viral transport media and vigorously mixed for one minute according to standard protocol, an aliquot was taken for culture, and the remaining sample was frozen. For this testing, a swab was dipped into the remaining sample and was assayed using the fluID test. An additional 100 μL aliquot was taken from each sample, the nucleic acid was purified, and a real time PCR assay for influenza A virus detection was run.

TABLE 6
Study results using a 4 line pRNA capture
system for detection of influenza A.

| | Culture & PCR+ | Culture− |
|---|---|---|
| fluID+ | 25 | 3 |
| fluID− | 2 | 91 |
| Sens. | 92.6% | =25/(25 + 2) |
| Spec. | 96.8% | =91/(91 + 3) |
| PPV | 89.3% | TP/(TP + FP) |
| NPV | 97.80% | TN/(TN + FN) |

Of the 5 culture−/fluID+ samples, 3 were confirmed positive based on the real time results. When these results are factored in, the sensitivity, specificity, positive predictive value (PPV), and negative predictive value (NPV) are 92.6%, 96.8%, 89.3%, and 97.8%, respectively. PPV is calculated as the total positives (TP) divided by the sum of the TP and the false negatives (FN). NPV is calculated as the total negatives (TN) divided by the sum of the TN and false negatives (FN). As can be seen in the two data sets, the identified conjugate pRNA:protein ratios improved assay performance.

Interference and specificity studies were also run with bacteria (n=10), viruses (n=10), and potential inhibitory substances (n=15). No cross reactivity or significant interference was detected during this testing. These results demonstrate that the fluID Rapid Influenza Test is a highly sensitive and specific assay for the detection and differentiation of influenza virus.

### Example 2

### Seasonal Assay Using Titered Cultured Virus

This study examines the analytical performance of both A and B analytes in the Seasonal assay using titered cultured virus. Each strain of virus had a TCID50 titer and each was diluted until the no signal was generated in the assay. Each dilution was tested using a commercially available point-of-care A and B Influenza assay kit as well as a PCR test. In one embodiment, the dilutional sensitivity results indicated that the A and B analytes are 2 to 3 logs more sensitive as compared to commercially available influenza A & B point-of-care assay, while being only 1 to 2 logs less sensitive than PCR.

### Example 3

### Examination of Levels of Viral Titer in Infected Patients

This study examined the analytical performance of a rapid influenza test using a system of the invention as compared to the Quidel QuickVue® system as well as PCR analysis. Both A and B analytes were assayed from different geographical locations. Each strain of virus had a TCID50 titer and each was diluted until the no signal was generated in the assay. Each dilution was tested using the commercially available Quidel QuickVue® kit as well as a PCR test. The dilutional sensitivity study indicated that the system of the invention is more sensitive in detection of A and B influenza target analytes versus commercially available influenza assay, while being only 1 to 2 logs less sensitive than PCR.

### Example 4

### Examination of Detection of H5 at Clinically Relevant Concentrations in Nasal Samples

This study examines the analytical performance of a rapid influenza targeting H5 analytes at clinically relevant concentrations in nasal samples. H1 and H3 samples were also tested. Each strain of virus had a TCID50 titer and each was diluted until the no signal was generated in the assay. Samples were detected at titers of down to 10².

### Example 5

### Comparison of Nasal Samples to PCR

This study examines the analytical performance of a rapid influenza test using a device of the invention on nasopharangyl samples compared against PCR. 164 samples were tested during a flu season. Of the 34 A+ influenza samples found positive by PCR, 100% of the samples were detected using a device of the invention. Of the 6 B+ influenza samples found positive by PCR, 100% of the samples were detected using a device of the invention. Of the 123 influenza samples found negative by PCR, a device of the present invention detected 99.2% of the samples as negative. Of the samples, there was one sample indeterminant by PCR.

### Example 6

### Retrospective Nasal Sample Detection

One hundred retrospectively collected nasal aspirate samples were tested and confirmed by culture. A device of the present invention was compared to commercially available systems. A device of the present invention detected 86-87% of the positive samples, whereas other commercial systems detected 69-80%.

While various embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 7

### Examination of Levels of Viral Titer Using Multiple Influenza Analytes

In this example, a test device is used to assay for different subtypes of influena virus. A test device is designed with a test strip having separate addressable lines 1980 to assay for A, H1, H3 and B analytes. An illustration of the test device is shown in FIG. 19. pRNA capture moiety partners 1960 (e.g., pRNAb, pRNAd, pRNAf, and pRNAh, that is 4 different sequences of pRNA) are immobilized to the test strip at different addressable lines 1980 (from left to right in FIG. 19). Sample from a sample collection device where the sample has been mixed with capture probe 1930 and detection probe 1910 is inserted into the test device at 1940. The capture probes have pRNA molecules 1950 each attached to an antibody that is specific for a viral antigen 1920. The different shaped viral antigens 1920 are shown diagramatically to indicate the presence of antigens from different viruses and strains of the same virus. The detection probes 1910 have antibodies specific for a viral antigen bound to a Europium label 1970. The sample is carried with wash buffer in the direction of capillary flow 1990. In one embodiment, a control line 1985 is provided to assess test performance. The control line has immobilized thereto rabbit anti-mouse antibodies that will bind to mouse antibodies generated against influenza A, H1, H3 and influenza B.

The pRNA molecules 1950 included in the capture moiety 1930 (pRNAa, pRNAc, pRNAe, and pRNAg) bind to their respective pRNA capture moiety partners 1960 (pRNAb, pRNAd, pRNAf, and pRNAh), thus capturing a complex with the viral antigen 1920 and detection moiety 1910. Each Analtye Binding Set (ABS) is designed for each of the analytes (i.e., influenza A, H1, H3, and B), wherein each of four different ABSs comprises in respective turn, a capture probe having a mouse anti-A antibody linked to a pRNA complementary to an immobilized pRNA on the first test zone and a detection probe of a mouse anti-influenza A antibody conjugated to a Europium label; a capture probe having mouse anti-H1 antibody linked to a pRNA that is complementary to an immobilized pRNA on the second test zone and a detection probe of a mouse anti-inflenza H1 antibody conjugated to a Europium label; a capture probe having a mouse anti-H3 antibody linked to a third pRNA that is complementary to a pRNA immobilized on the third test zone and a detection probe of a mouse anti-influenza H3 antibody conjugated to a Europium label; and a fourth capture probe having a mouse anti-B antibody linked to a pRNA complementary to an immobilized pRNA on a fourth test zone and a detection probe of a mouse anti-influenza B antibody conjugated to a Europium label.

Following capillary flow, the test device is tested for Europium binding at the different addressable lines 1980 for the detection of different influenza subtypes.

### Example 8

### Examination of Levels of Viral Titer Using Multiple Influenza Analytes

In this example, a test device is used to assay for different subtypes of influenza virus. A test device is designed with a test strip having separate addressable lines to assay for influenza A, H1, H3, H5, and B analytes. The device utilizes 5 analyte binding sets of probe conjugates and detection probes for reaction with the sample in the sample collection device before application to the test device. Analyte binding set 1 comprises a capture probe of an antibody to influenza A conjugated to a pRNA and a detection probe comprising an second antibody to Influenza A coupled to a Europium label. Set 2 includes a capture probe comprising an antibody to H1 conjugated to biotin and a detection probe comprising a second antibody to H1 coupled to a Europium label. Analyte binding set 3 comprises a capture probe of an antibody to H3 conjugated to a pRNA and a detection probe comprising a second antibody to H3 coupled to a Europium label.

Analyte binding set 4 comprises a capture probe of an antibody to H5 conjugated to streptavidin and a detection probe comprising a second antibody to H5 coupled to a Europium label. Analyte binding set 5 comprises a capture probe of an antibody to influenza B conjugated to a pRNA and a detection probe comprising a second antibody to Influenza B coupled to a Europium label. At each of addressable lines 1, 3, and 5, a different pRNA is immobilized, the pRNA at line 1 capable of capturing an immunocomplex for influenza A; line 3 having immobilized a pRNA capable of capturing an immunocomplex for H3 and line 5 having immobilized a pRNA capable of capturing an immunocomplex for influenza B. At addressable line 2 is immobilized streptavidin capable of capturing an immunocomplex to H1, and at addressable line 4 is immobilized biotin capable of capturing an immunocomplex of H5.

The device does not have adjacent addressable lines with capture moiety partners the same category (e.g. pRNA or avidin/streptavidin). A patient sample is collected on a sample collection implement and inserted into the sample collection device, seating the upper chamber onto the sample collection tube and sealing the device. The fluid in the upper chamber is released so the liquid flows over the swab or collection implement and washes over it, releasing the sample from the collection implement into the liquid and flows down into the lower chamber of the sample collection tube. The fluid containing the patient sample mixes with the 5 analyte binding sets in the lower chamber of the sample collection device. If analytes of interest are present the sample reacts and forms immunocomplexes.

The dispensing tip of the sample collection device is inserted into the port of the test device and the sample mixture containing any immunocomplexes is delivered to the test device. After delivery of the sample mixture, the wash buffer of the test device is released.

The sample mixture is carried by wash buffer in the direction of capillary flow. Following capillary flow, the test device is tested for Europium binding at different addressable lines for the detection of different influenza subtypes.

### Example 9

### Striping of pRNA Conjugates onto Test Device

In this example, pRNA conjugates are prepared and striped onto a nitrocellulose strip for use in a test device of the present invention.

Materials and Methods:

Chemicals. EZ-Link-NHS-Chromogenic Biotin is purchased from Pierce Chemical Co. (Rockford, Ill.). Nitrocellulose membrane (SA3J107107) is purchased from Millipore, Streptavidin Europium (SAEU) latex particles (Catalogue number 2947-0701) is purchased from Thermofisher Scientific (Seradyne).

The following pRNA oligomers are synthesized: 4a9-Indole, ATGCDCTTC (where D represents the indole base in the sequence); 4b8-Indole, GAADGCAT; 5a8 TGATGGAC; 5b9-Indole, GTCDCATCA; 6a6, CAGTAG; 6b6, CTACTG; 8a6, GACTCT; and 8b6, AGAGTC.

Extraction reagent: 50 mM Tris, pH 8.5; 0.75 M NaCl; 1.5% Bovine Serum Albumin; 0.75% Digested Casein; 25 μg/mL Mouse IgG; 1.5% saponin; 0.37% Lauryl Sulfobetaine 3-12; 50 μl/mL Gentamicin; 0.095% Sodium Azide and 0.0045% silicone antifoam. Extraction reagent bulbs are filled with 195 μl of extraction reagent.

Wash buffer: 20% w/v sucrose, 50 mM Tris, pH 8.5; 0.75 M NaCl; 1.5% Bovine Serum Albumin; 0.75% Digested Casein; 1.5% saponin; 0.37% Lauryl Sulfobetaine 3-12; 50 μl/mL Gentamicin; 0.095% Sodium Azide and 0.0045% silicone antifoam. Wash buffer packets are filled with 110 μl of wash buffer.

Antibodies. The AAH5 anti-influenza A nucleoprotein monoclonal antibody is purchased from Meridian (Cincinnati, Ohio). The M4090913 anti-ingluenza A nucleoprotein and the M2110171 anti-influenza B nucleoprotein monoclonal antibodies are purchased from Fitzgerald Industries (Concord, Mass.). The 2/3 anti-influenza B nucleoprotein monoclonal antibody is purchased from HyTest Ltd, (Turku, Finland). The 9D5 and 4C10 anti-H1 hemagglutinin and the 4D1, 8H5 and 2F10 anti-H5 hemagglutinin monoclonal antibodies are purchased from HX Diagnostics (Emeryville, Calif.). The 2H11 and 1F4 anti-H3 hemagglutinin and the 2-199C anti-cytochrome C monoclonal antibodies are produced by BioProcessing Inc, (Portland, Me.). Control line antibody Rabbit anti-Mouse IgG Fc Fragment specific is from Jackson ImmunoResearch Laboratories (West Grove, Pa.).

Conjugations:

Biotin conjugations are performed in 75 mM Sodium Borate buffer, pH 9.0, at a biotin:antibody ratio of 2:1 for 2 hours at room temperature. Biotin conjugates are purified to remove any high molecular weight contaminants by size exclusion chromatography using Sephacryl 5300. pRNA conjugations to antibody or other proteins are performed reacting activated pRNA with antibody in 75 mM Sodium Borate buffer, pH 9.0, at room temperature for 14-18 hours. pRNA conjugates are purified to remove any high molecular weight contaminants by size exclusion chromatography using Sephacryl S300. Biotinylated antibodies are coupled to SAEU particles by incubating two volumes of biotinylated antibody at 0.15 mg/ml with 1 volume of 0.2% SAEU particles for 2 hours at room temperature with agitation. Unbound streptavidin is blocked for an additional 2 hours with one volume of 10 uM biotin. The coupled particles are washed by hollow fiber diafiltration. The concentration of the washed beads is determined by fluorescence using a 0.2% SAEU particle standard.

Lyophilized Reagent Pellets:

Reagents are lyophilized as 20 μl pellets by dispensing 20 μl of reagent formulation into liquid nitrogen. The frozen reagent pellets are then lyophilized and kept dry until used. Reagent formulations used are as follows:

pRNA pellets: pRNA-antibody conjugates; A, B, H1, H3, H5 0.05-0.5 ug each per 20 μl reagent pellet; 10 mM Tris, pH 8.0; 1% BSA; and 0.3 M Trehalose.

Europium pellets: Europium conjugates; A, B, H1, H3 and H5 1.0-10 ug Euopium-antibody beads per 20 μl reagent pellet; 10 mM Tris, pH 8.0; 1% BSA; and 0.3 M Trehalose.

Tris (2-carboxyethylphosphine HCl (TCEP) 20 μl pellets: 17 mM TCEP, 10 mM Tris, pH 8.0; 1% BSA; and 0.3 M Trehalose.

Application of Test Line pRNA Conjugates to Nitrocellulose.

Test Line pRNAs are conjugated to the 2-199C monoclonal antibody and adjusted to 1.5 mg/ml in PBS buffer containing 3% methanol. The Test line conjugates are dispensed onto the nitrocellulose at a rate of 0.075 μl/mm using an Imagene Technology IsoFlow™ Dispenser. The control line Rabbit anti-Mouse antibody is applied at a concentration of 1.2 mg/ml without the methanol. The application order is 4b9-In conjugate, 8a6 conjugate, 6b6 conjugate, 5b9-In conjugate and control line.

```
SEQ ID NO: 1 8H5 Vh Nucleotide sequence
caggttcagc tgcagcagtc tggagctgag ctgatgaagc ctggggcctc agtgaagata
tcctgcaagg ctactggcta cactttcagt aactactgga tagagtggat aaagcagagg
cctggacatg gccttgagtg gattggagag attttacctg gaagcgatag aacaaactac
aatgggaagt tcaagggcaa ggccacattc actgcagata catcctccaa cacagcccac
atgcaactca gtagcctgac atctgaggac tctgccgtct attactgtgc aaatagatac
gacgggtatt attttggttt ggattactgg ggtcaaggaa cctcagtcgc cgtctcctca
gcc
SEQ ID NO: 2
Gln Val Gln Leu Gln Gln Ser Gly Ala Glu Leu Met Lys Pro Gly Ala
1 5 10 15
Ser Val Lys Ile Ser Cys Lys Ala Thr Gly Tyr Thr Phe Ser Asn Tyr
                        20 25 30
Trp Ile Glu Trp Ile Lys Gln Arg Pro Gly His Gly Leu Glu Trp Ile
                35 40 45
Gly Glu Ile Leu Pro Gly Ser Asp Arg Thr Asn Tyr Asn Gly Lys Phe
        50 55 60
Lys Gly Lys Ala Thr Phe Thr Ala Asp Thr Ser Ser Asn Thr Ala His
65 70 75 80
Met Gln Leu Ser Ser Leu Thr Ser Glu Asp Ser Ala Val Tyr Tyr Cys
                                85 90 95
Ala Asn Arg Tyr Asp Gly Tyr Tyr Phe Gly Leu Asp Tyr Trp Gly Gln
                        100 105 110
Gly Thr Ser Val Ala Val Ser Ser Ala
SEQ ID NO: 3 8H5 Vk Nucleotide sequence
gaaatcgtgc tcacccagtc tccagcaatc atgtctgcat ctctagggga gaaggtcacc
atgagctgca gggccagctc aagtgtaaat ttcgtttact ggtaccagca gaggtcagat
gcctccccca aactattgat ttactattca tccaacctgg ctcctggagt cccacctcgc
ttcagtggca gtgggtctgg gaactcttat tctctcacaa tcagcggctt ggagggtgaa
gatgctgcca cttattactg ccagcacttt actagttccc cgtacacgtt cggagggggg
accaacctgg aaataaaacg g
SEQ ID NO: 4 8H5 Vk Amino Acid sequence
Glu Ile Val Leu Thr Gln Ser Pro Ala Ile Met Ser Ala Ser Leu Gly
1 5 10 15
Glu Lys Val Thr Met Ser Cys Arg Ala Ser Ser Ser Val Asn Phe Val
                        20 25 30
Tyr Trp Tyr Gln Gln Arg Ser Asp Ala Ser Pro Lys Leu Leu Ile Tyr
                35 40 45
Tyr Ser Ser Asn Leu Ala Pro Gly Val Pro Pro Arg Phe Ser Gly Ser
        50 55 60
Gly Ser Gly Asn Ser Tyr Ser Leu Thr Ile Ser Gly Leu Glu Gly Glu
65 70 75 80
Asp Ala Ala Thr Tyr Tyr Cys Gln His Phe Thr Ser Ser Pro Tyr Thr
                                85 90 95
Phe Gly Gly Gly Thr Asn Leu Glu Ile Lys Arg
                        100 105
SEQ ID NO: 5 3C8 Vh Nucleotide sequence
cagatccagt tggtgcagtc tggacctgag ctgaagaagc ctggagagac agtcaagatc
tcctgcaagg cctctgggta cagcttcaca aactatggaa tgaactgggt gaagcaggct
ccaggaaagg gtctaaagtg gatgggctgg ataaacacct acaccggaga gccagcctat
gctgatgact tcaagggacg gtttgccttc tctctggaaa cctctgccag cactgcctat
ttgcagatca acaacctcaa aaatgaggac acggctacat atttctgtgc aagatggaat
agagatgcta tggactactg gggtcaagga acctcggtca ccgtatctag c
SEQ ID NO: 6 3C8 Vh Amino Acid sequence
Gln Ile Gln Leu Val Gln Ser Gly Pro Glu Leu Lys Lys Pro Gly Glu
1 5 10 15
Thr Val Lys Ile Ser Cys Lys Ala Ser Gly Tyr Ser Phe Thr Asn Tyr
                        20 25 30
Gly Met Asn Trp Val Lys Gln Ala Pro Gly Lys Gly Leu Lys Trp Met
                35 40 45
Gly Trp Ile Asn Thr Tyr Thr Gly Glu Pro Ala Tyr Ala Asp Asp Phe
        50 55 60
Lys Gly Arg Phe Ala Phe Ser Leu Glu Thr Ser Ala Ser Thr Ala Tyr
65 70 75 80
Leu Gln Ile Asn Asn Leu Lys Asn Glu Asp Thr Ala Thr Tyr Phe Cys
                                85 90 95
Ala Arg Trp Asn Arg Asp Ala Met Asp Tyr Trp Gly Gln Gly Thr Ser
                        100 105 110
Val Thr Val Ser Ser
                115
SEQ ID NO: 7 3C8 VK Nucleotide sequence
gacattgtgc tgacccaatc tccagcttct ttggctgtgt ctcttgggca gagggccacc
atatcctgca gagccagtga aagtgttgat agttctgaca atagtcttat gcactggtac
cagcagaaac caggacagcc acccaaactc ctcatctatc gtgcatccaa cctagaatct
gggatccctg ccaggttcag tggcagtggg tctaggacag acttcaccct caccattaat
cctgtggagg ctgatgatgt tgcaacctat tactgtcagc aaagtattgg ggatcctccg
tacacgttcg gaggggggac caagctggaa ataaaacgg
SEQ ID NO: 8 3C8 VK Amino Acid sequence
Asp Ile Val Leu Thr Gln Ser Pro Ala Ser Leu Ala Val Ser Leu Gly
1 5 10 15
Gln Arg Ala Thr Ile Ser Cys Arg Ala Ser Glu Ser Val Asp Ser Ser
                        20 25 30
Asp Asn Ser Leu Met His Trp Tyr Gln Gln Lys Pro Gly Gln Pro Pro
                35 40 45
Lys Leu Leu Ile Tyr Arg Ala Her Asn Leu Glu Ser Gly Ile Pro Ala
        50 55 60
Arg Phe Ser Gly Ser Gly Ser Arg Thr Asp Phe Thr Leu Thr Ile Asn
65 70 75 80
Pro Val Glu Ala Asp Asp Val Ala Thr Tyr Tyr Cys Gln Gln Ser Ile
                                85 90 95
Gly Asp Pro Pro Tyr Thr Phe Gly Gly Gly Thr Lys Leu Glu Ile Lys
                        100 105 110
Arg
SEQ ID NO: 9 10F7 Vh Nucleotide sequence
caggtccaac tgcagcagcc tggggctgaa cttgtgaagc ctggggcttc agtgaagctg
tcctgcaagg cttctggcta caccttcacc agctactgga tgcactgggt gaagcagagg
cctggacagg gccttgagtg gatcggagag attgatcctt ctgattctta tactaactac
aatcagaagt tcaagggcaa ggccacattg actgtagaca aatcctccag cacagcctac
atgcagctca gcagcctgac atctgaggac tctgcggtct attactgtgc aagggggggt
acaggagact ttcactatgc tatggactac tggggtcaag gcacctcggt caccgtatca
tcg
SEQ ID NO: 10 10F7 Vh Amino Acid sequence
Gln Val Gln Leu Gln Gln Pro Gly Ala Glu Leu Val Lys Pro Gly Ala
1 5 10 15
Ser Val Lys Leu Ser Cys Lys Ala Ser Gly Tyr Thr Phe Thr Ser Tyr
                        20 25 30
Trp Met His Trp Val Lys Gln Arg Pro Gly Gln Gly Leu Glu Trp Ile
                35 40 45
Gly Glu Ile Asp Pro Ser Asp Ser Tyr Thr Asn Tyr Asn Gln Lys Phe
        50 55 60
Lys Gly Lys Ala Thr Leu Thr Val Asp Lys Ser Ser Ser Thr Ala Tyr
65 70 75 80
Met Gln Leu Ser Ser Leu Thr Ser Glu Asp Ser Ala Val Tyr Tyr Cys
                                85 90 95
Ala Arg Gly Gly Thr Gly Asp Phe His Tyr Ala Met Asp Tyr Trp Gly
                        100 105 110
Gln Gly Thr Ser Val Thr Val Ser Ser
                115 120
SEQ ID NO: 11 - 10F7 VK Nucleotide sequence
gacatcctga tgacccaatc tccatcctcc atgtctgtat ctctgggaga cacagtcagc
atcacttgcc atgcaagtca gggcattagc agtaatatag ggtggttgca gcagaaacca
gggaaatcat ttaagggcct gatctatcat ggaaccaact tggaagatgg agttccatca
aggttcagtg gcagtggatc tggagcagat tattctctca ccatcagcag cctggaatct
gaagattttg cagactatta ctgtgtacag tatgttcagt tcccgtacac gttcggaggg
ggcaccaagc tggaaatcaa acgg
SEQ ID NO: 12 10F7 VK Amino Acid sequence
Asp Ile Leu Met Thr Gln Ser Pro Ser Ser Met Ser Val Ser Leu Gly
1 5 10 15
Asp Thr Val Ser Ile Thr Cys His Ala Ser Gln Gly Ile Ser Ser Asn
                        20 25 30
Ile Gly Trp Leu Gln Gln Lys Pro Gly Lys Ser Phe Lys Gly Leu Ile
                35 40 45
Tyr His Gly Thr Asn Leu Glu Asp Gly Val Pro Ser Arg Phe Ser Gly
        50 55 60
Ser Gly Ser Gly Ala Asp Tyr Ser Leu Thr Ile Ser Ser Leu Glu Ser
65 70 75 80
Glu Asp Phe Ala Asp Tyr Tyr Cys Val Gln Tyr Val Gln Phe Pro Tyr
                                85 90 95
Thr Phe Gly Gly Gly Thr Lys Leu Glu Ile Lys Arg
                        100 105
SEQ ID NO: 13. Artificial sequence/Unknown Organism
catgggatgc tgccggtgta t
SEQ ID NO: 14. Artificial Sequence/Unknown Organism
aattctgggc cttggctgac g
SEQ ID NO: 15. Artificial Sequence/Unknown Organism
tggccgcctc tgtcgaagaa g
SEQ ID NO: 16. 4D1 VH Nucleotide sequence
caggtccaac tgcagcagcc tggggctgag cttgtgaagc ctggggcttc agtgaacctg
tcctgtaagg cttctggcta caccttcacc agctactgga tgcactgggt gaagcagagg
cctggacaag gccttgagtg gatcggagag attgatcctt ctgatagttt tactacctac
aatcaaaact tcaaagacag ggccacattg actgtagaca aatcatccag cacagcctac
atgcagctca gaagtctgac atctgaggac tctgcggtct attactgtgc cagggggggt
ccaggagact ttcgctatgc tatggattac tggggccaag gcacctcggt caccgtctcc
tca
SEQ ID NO: 17 - 4D1 VH Amino Acid sequence
Gln Val Gln Leu Gln Gln Pro Gly Ala Glu Leu Val Lys Pro Gly Ala
1 5 10 15
Ser Val Asn Leu Ser Cys Lys Ala Ser Gly Tyr Thr Phe Thr Ser Tyr
                        20 25 30
Trp Met His Trp Val Lys Gln Arg Pro Gly Gln Gly Leu Glu Trp Ile
                35 40 45
Gly Glu Ile Asp Pro Ser Asp Ser Phe Thr Thr Tyr Asn Gln Asn Phe
        50 55 60
Lys Asp Arg Ala Thr Leu Thr Val Asp Lys Ser Ser Ser Thr Ala Tyr
65 70 75 80
Met Gln Leu Arg Ser Leu Thr Ser Glu Asp Ser Ala Val Tyr Tyr Cys
                                85 90 95
Ala Arg Gly Gly Pro Gly Asp Phe Arg Tyr Ala Met Asp Tyr Trp Gly
                        100 105 110
Gln Gly Thr Ser Val Thr Val Ser Ser
                115 120
SEQ ID NO: 18 - 4D1 VK Nucleotide sequence
gacatcctga tgacccaatc tccatcctcc atgtctgtat ctctgggaga cacagtcagc
atcacttgcc atgcaagtca gggcattagc agtaatatag ggtggttgca gcagaaacca
gggaaatcat ttaagggcct gatctatcat ggaaccaact tggaagatgg agttccatca
aggttcagtg gcagtggatc tggagcagat tattctctca ccatcagcag cctggaatcc
gaagactttg cagactatta ctgtgtacag tatgttcagt ttccctacac gttcggaggg
gggaccaagc tggaaataaa acgggct
SEQ ID NO: 19 - 4D1 Vk Amino Acid sequence
Asp Ile Leu Met Thr Gln Ser Pro Ser Ser Met Ser Val Ser Leu Gly
1 5 10 15
Asp Thr Val Ser Ile Thr Cys His Ala Ser Gln Gly Ile Ser Ser Asn
                        20 25 30
Ile Gly Trp Leu Gln Gln Lys Pro Gly Lys Ser Phe Lys Gly Leu Ile
                35 40 45
Tyr His Gly Thr Asn Leu Glu Asp Gly Val Pro Ser Arg Phe Ser Gly
        50 55 60
Ser Gly Ser Gly Ala Asp Tyr Ser Leu Thr Ile Ser Ser Leu Glu Ser
65 70 75 80
Glu Asp Phe Ala Asp Tyr Tyr Cys Val Gln Tyr Val Gln Phe Pro Tyr
                                85 90 95
Thr Phe Gly Gly Gly Thr Lys Leu Glu Ile Lys Arg Ala
                        100 105
SEQ ID NO: 20 - 3G4 VH Nucleotide sequence
caggtccaac tgcagcagtc tggggctgag ctggtgaggc ctggggtctc agtgaagatt
tcctgcaagg gttctggcta cacattcact gattatgcta tgcattgggt gaagcagagt
catgcaaaga gtctagagtg gattggactt attaatactg actatggtga tactacttac
aaccagaagt tcaagggcaa ggccacaatg actgtagaca aatcctccaa cacagcctat
atggaacttg ccagactgac atctgaggat tctgccatct attactgtgc aagatcggac
tatgattact atttctgtgg tatggactac tggggtcaag gaaccacggt caccgaatct
cta
SEQ ID NO: 21 - 3G4 VH Amino Acid sequence
Gln Val Gln Leu Gln Gln Ser Gly Ala Glu Leu Val Arg Pro Gly Val
1 5 10 15
Ser Val Lys Ile Ser Cys Lys Gly Ser Gly Tyr Thr Phe Thr Asp Tyr
                        20 25 30
Ala Met His Trp Val Lys Gln Ser His Ala Lys Ser Leu Glu Trp Ile
                35 40 45
Gly Leu Ile Asn Thr Asp Tyr Gly Asp Thr Thr Tyr Asn Gln Lys Phe
        50 55 60
Lys Gly Lys Ala Thr Met Thr Val Asp Lys Ser Ser Asn Thr Ala Tyr
65 70 75 80
Met Glu Leu Ala Arg Leu Thr Ser Glu Asp Ser Ala Ile Tyr Tyr Cys
                                85 90 95
Ala Arg Ser Asp Tyr Asp Tyr Tyr Phe Cys Gly Met Asp Tyr Trp Gly
                        100 105 110
Gln Gly Thr Thr Val Thr Glu Ser Leu
                115 120
SEQ ID NO: 24 - 2F2 VH Nucleotide sequence
caggtgcagc tgaaggagtc aggacctggc ctggtggcgc cctcacagcg cctgtccatc
acatgcaccg tctcagggtt ctcattaacc ggctatggtg tacactggat tcgccagtct
ccaggaaagg gtctggagtg gctgggaatg atatgggctg agggaagaac cgactataat
tcagttctca aatccagact gagcatcaat aaggacaatt ccaggagcca agttttctta
gaaatgaaca gtctgcaaac tgatgacaca gccaggtact actgtgccag agaggtgatt
actacggaag cctggtactt cgatgtctgg ggccaaggaa cctcggtcac cgaatct
SEQ ID NO: 25 - 2F2 VH Amino Acid sequence
Gln Val Gln Leu Lys Glu Ser Gly Pro Gly Leu Val Ala Pro Ser Gln
1 5 10 15
Arg Leu Ser Ile Thr Cys Thr Val Ser Gly Phe Ser Leu Thr Gly Tyr
                        20 25 30
Gly Val His Trp Ile Arg Gln Ser Pro Gly Lys Gly Leu Glu Trp Leu
                35 40 45
Gly Met Ile Trp Ala Glu Gly Arg Thr Asp Tyr Asn Ser Val Leu Lys
        50 55 60
Ser Arg Leu Ser Ile Asn Lys Asp Asn Ser Arg Ser Gln Val Phe Leu
65 70 75 80
Glu Met Asn Ser Leu Gln Thr Asp Asp Thr Ala Arg Tyr Tyr Cys Ala
                                85 90 95
Arg Glu Val Ile Thr Thr Glu Ala Trp Tyr Phe Asp Val Trp Gly Gln
                        100 105 110
Gly Thr Ser Val Thr Glu Ser
                115
SEQ ID NO: 26 - 2F2 VK Nucleotide sequence
gacattgtga tgactcagtc tccagccacc ctgtctgtga ctccaggaga tagagtctct
ctttcctgca gggccagcca gagtattagc gactacttat actggtatca acaaaaatca
catgagtctc caaggcttct catcaaatat gcttcccaat ccatctctgg gatcccctcc
agattcagtg gcagtggatc agggtcagat ttcactctca ctatcaacag tgtggaacct
gaagatgttg gaatgtatta ctgtcaaaat ggtcacacct ttccgctcac gttcggtgct
ggcaccaagc tggaaatcaa acgg
SEQ ID NO: 27 - 2F2 VK Amino Acid sequence
Asp Ile Val Met Thr Gln Ser Pro Ala Thr Leu Ser Val Thr Pro Gly
1 5 10 15
Asp Arg Val Ser Leu Ser Cys Arg Ala Ser Gln Ser Ile Ser Asp Tyr
                        20 25 30
Leu Tyr Trp Tyr Gln Gln Lys Ser His Glu Ser Pro Arg Leu Leu Ile
                35 40 45
Lys Tyr Ala Ser Gln Ser Ile Ser Gly Ile Pro Ser Arg Phe Ser Gly
        50 55 60
Ser Gly Ser Gly Ser Asp Phe Thr Leu Thr Ile Asn Ser Val Glu Pro
65 70 75 80
Glu Asp Val Gly Met Tyr Tyr Cys Gln Asn Gly His Thr Phe Pro Leu
                                85 90 95
Thr Phe Gly Ala Gly Thr Lys Leu Glu Ile Lys Arg.
```

1. A system for detecting the presence, absence, or level of one or more analytes in a sample, comprising:
a sample collection device configured for mixing a patient sample with one or more immunoreagents to form one or more capturable and detectable immunocomplex(es); and
a test device comprising a lateral flow membrane for capturing the immunocomplex(es), wherein the sample collection device and the test device are configured to form an air-tight seal and/or to release the sample through a split septum onto the lateral flow membrane.

2. A sample collection device comprising a body comprising:
(a) an upper chamber comprising an upper sealed compartment containing one or more solutions, and at least one breakable seal;
(b) a sample collection implement in fluid communication with said upper chamber;
(c) a sample receiving tube in fluid communication with the upper chamber, wherein said tube is composed of a rigid material; wherein the upper chamber and sample receiving tube are configured to form an air-tight seal when coupled together;
(d) a lower chamber in fluid communication with the sample receiving tube containing one or more reagents; and
(e) wherein said reagents comprise a plurality of analyte binding sets, wherein each of the sets comprises:
(i) a capture probe comprising: (1) a binding moiety that is capable of specifically binding a target analyte, and (2) a capture moiety partner; and
(ii) a detection probe comprising: (1) a second binding moiety that is capable of specifically binding a target analyte and (2) a label; and
(iii) wherein each of said plurality of analyte binding sets is designed to bind a different target analyte.

3. The sample collection device of claim 1 wherein the capture moiety partner is selected from a group consisting of an oligonucleotide, avidin, streptavidin, pyranosyl RNA (pRNA), aptamer or a combination thereof.

4. The sample collection device of claim 2 wherein the pRNA comprises a sequence selected from the group consisting of SEQ ID NO: 120 to SEQ ID NO: 126.

5. The sample collection device of claim 1 wherein said target analyte is an influenza virus or component thereof.

6. The sample collection device of claim 1 wherein said extraction solution or said reagents comprises an extraction agent.

7. The sample collection device of claim 1 further comprising a mesh membrane that separates said reagents in the lower chamber from a remaining portion of the sample receiving tube.

8. The sample collection device of claim 7 wherein said reagents further comprises a dye that is capable of indicating that said sample is sufficiently mixed with said reagents.

9. The sample collection appliance of claim 1 wherein said upper chamber comprises at least two subchambers.

10. The sample collection device of claim 9 wherein each of said subchambers contain a solution.

11. The sample collection device of claim 1 wherein said contact is capable of forming a positive pressure differential relative to ambient pressure that is capable of expelling a solution from said sample collection device.

12. The sample collection device of claim 1 wherein said lower chamber further comprises a split septum that is capable of releasing a solution when perforated.

13. The sample collection device of claim 12 wherein the split septum comprises neoprene.

14. The sample collection device of claim 1 further comprising a first and a second indicator present on said sample receiving tube, wherein said indicators provide an indication of proper contact between said upper chamber and said sample receiving tube.

15. The sample collection device of claim 1 wherein said sample collection implement is attached to said upper chamber.

16. A test device comprising a body comprising:
(a) a lateral flow membrane in the body;
(b) a chamber positioned upstream of a gap present between said chamber and said lateral flow membrane;
(c) one or more control line; and
(d) a plurality of addressable lines, each line comprising a capture moiety partner, wherein said capture moiety partner is selected from a plurality of molecule categories and any two adjacent addressable lines comprise capture moiety partners that are of a different category.

17-20. (canceled)

21. A method for detecting one or more target analyte comprising:
(a) obtaining a sample from a subject and mixing said sample inside a sample collection device, wherein the sample collection device comprises:
(i) an upper chamber comprising an upper sealed compartment containing one or more solutions, and at least one breakable seal;
(ii) a sample collection implement in fluid communication with said upper chamber;
(iii) a sample receiving tube in fluid communication with the upper chamber, wherein said tube is composed of a rigid material; wherein the upper chamber and sample receiving tube are configured to form an air-tight seal when coupled together;
(iv) a lower chamber in fluid communication with the sample receiving tube containing one or more reagents; and wherein said reagents comprise a plurality of analyte binding sets, wherein each of the sets comprises:
(1) a capture probe comprising: (1) a binding moiety that is capable of specifically binding a target analyte, and (2) a capture moiety partner; and
(2) a detection probe comprising: (1) a second binding moiety that is capable of specifically binding a target analyte and (2) a label; and wherein each of said plurality of analyte binding sets is designed to bind a different target analyte;
(b) breaking the seal of said upper chamber to release one or more solutions into said sample receiving tube, thereby releasing said sample from said sample collection implement and mixing said sample with said reagents;
(c) applying the mixture formed in (b) to a test strip comprising a plurality of addressable lines, wherein each of said addressable lines comprises an immobilized capture moiety partner that is capable of binding to a different said capture probe in (a)(4), whereby each addressable line is configured to bind a different target analyte; and
(d) determining if a label is present in one or more addressable line; and thereby detecting if the sample contains one or more target analyte.

22-28. (canceled)

29. A method of detecting one or more target analytes in a sample, comprising: applying a sample comprising an immunocomplex to a lateral flow device comprising one or more addressable lines, wherein at least one of said one or more addressable lines comprises a pRNA capture moiety partner bound thereto, and wherein said pRNA is selected from a group consisting of SEQ ID NO: 120 to SEQ ID NO:126.

30. A kit comprising:
(a) a lateral flow device comprising a bibulous strip comprising (i) a sample application zone, (ii) multiple detection zones comprising two or more addressable lines, wherein each of said addressable lines comprises an immobilized reagent, wherein at least two adjacent of said addressable lines comprises a different immobilized pRNA selected from a group consisting of SEQ ID NO: 120 to SEQ ID NO:126;
(b) a plurality of specific binding reagents wherein said reagents comprise two or more specific binding pairs, wherein each of said pairs is capable of binding a different target analyte, and wherein each pair comprises: (1) a first conjugate comprising: (i) a binding agent capable of specifically binding an analyte and (ii) a capture reagent that is capable of specifically binding an immobilized reagent present on one of said addressable lines; and (2) a second conjugate comprising: (i) a binding agent capable of specifically binding said analyte of (b)(1) and (ii) a detectable label.

31. A method for detecting one or more target analyte comprising:
(a) obtaining a sample from a subject and placing said sample inside a sample collection device;
(b) releasing one or more solutions to mix said sample with said solutions;
(c) applying the mixture formed in (b) to a test strip; and
(d) determining if a label is present;
wherein the sensitivity of the method for detecting one or more target analyte is at least 70%.

32. The method of claim 31, wherein the sample collection device comprises:
(a) an upper chamber comprising an upper sealed compartment containing one or more solutions, and at least one breakable seal;
(b) a sample collection implement in fluid communication with said upper chamber;
(c) a sample receiving tube in fluid communication with the upper chamber, wherein said tube is composed of a rigid material; wherein the upper chamber and sample receiving tube are configured to form an air-tight seal when coupled together;
(d) a lower chamber in fluid communication with the sample receiving tube containing one or more reagents; and wherein said reagents comprise a plurality of analyte binding sets, wherein each of the sets comprises:
(i) a capture probe comprising: (1) a binding moiety that is capable of specifically binding a target analyte, and (2) a capture moiety partner; and
(ii) a detection probe comprising: (1) a second binding moiety that is capable of specifically binding a target analyte and (2) a label; and wherein each of said plurality of analyte binding sets is designed to bind a different target analyte.

33-36. (canceled)

37. A method for detecting one or more target analyte comprising:
(a) obtaining a sample from a subject and placing said sample inside a sample collection device;
(b) releasing one or more solutions to mix said sample with said solutions;
(c) applying the mixture formed in (b) to a test strip; and
(d) determining if a label is present;
wherein the specificity of the method for detecting one or more target analyte is at least 70%.

38. The method of claim 37, wherein the sample collection device comprises:
(a) an upper chamber comprising an upper sealed compartment containing one or more solutions, and at least one breakable seal;
(b) a sample collection implement in fluid communication with said upper chamber;
(c) a sample receiving tube in fluid communication with the upper chamber, wherein said tube is composed of a rigid material; wherein the upper chamber and sample receiving tube are configured to form an air-tight seal when coupled together;
(d) a lower chamber in fluid communication with the sample receiving tube containing one or more reagents; and wherein said reagents comprise a plurality of analyte binding sets, wherein each of the sets comprises:
(i) a capture probe comprising: (1) a binding moiety that is capable of specifically binding a target analyte, and (2) a capture moiety partner; and
(ii) a detection probe comprising: (1) a second binding moiety that is capable of specifically binding a target analyte and (2) a label; and wherein each of said plurality of analyte binding sets is designed to bind a different target analyte.

39-44. (canceled)


SEQUENCE LISTING (126 sequences)

SEQ ID NO: 1 - DNA, 363 bp, Mus sp.
caggttcagctgcagcagtctggagctgagctgatgaagcctggggcctcagtgaagatatcctgcaaggctactggctacactttcagtaactactggatagagtggataaagcagaggcctggacatggccttgagtggattggagagattttacctggaagcgatagaacaaactacaatgggaagttcaagggcaaggccacattcactgcagatacatcctccaacacagcccacatgcaactcagtagcctgacatctgaggactctgccgtctattactgtgcaaatagatacgacgggtattattttggtttggattactggggtcaaggaacctcagtcgccgtctcctcagcc

SEQ ID NO: 2 - protein, 123 aa, Mus sp.
QVQLQQSGAELMKPGASVKISCKATGYTFSNYWIEWIKQRPGHGLEWIGEILPGSDRTNYNGKFKGKATFTADTSSNTAHMQLSSLTSEDSAVYYCALANRYDGYYFGLDYWGQGTSVAVSSA

SEQ ID NO: 3 - DNA, 321 bp, Mus sp.
gaaatcgtgctcacccagtctccagcaatcatgtctgcatctctaggggagaaggtcaccatgagctgcagggccagctcaagtgtaaatttcgtttactggtaccagcagaggtcagatgcctcccccaaactattgatttactattcatccaacctggctcctggagtcccacctcgcttcagtggcagtgggtctgggaactcttattctctcacaatcagcggcttggagggtgaagatgctgccacttattactgccagcactttactagttccccgtacacgttcggaggggggaccaacctggaaataaaacgg

SEQ ID NO: 4 - protein, 107 aa, Mus sp.
EIVLTQSPAIMSASLGEKVTMSCRASSSVNFVYWYQQRSDASPKLLIYYSSNLAPGVPPRFSGSGSGNSYSLTISGLEGEDAATYYCQHFTSSPYTFGGGTNLEIKR

SEQ ID NO: 5 - DNA, 351 bp, Mus sp.
cagatccagttggtgcagtctggacctgagctgaagaagcctggagagacagtcaagatctcctgcaaggcctctgggtacagcttcacaaactatggaatgaactgggtgaagcaggctccaggaaagggtctaaagtggatgggctggataaacacctacaccggagagccagcctatgctgatgacttcaagggacggtttgccttctctctggaaacctctgccagcactgcctatttgcagatcaacaacctcaaaaatgaggacacggctacatatttctgtgcaagatggaatagagatgctatggactactggggtcaaggaacctcggtcaccgtatctagc

SEQ ID NO: 6 - protein, 119 aa, Mus sp.
QIQLVQSGPELKKPGETVKISCKASGYSFTNYGMNWVKQAPGKGLKWMGWINTYTGEPAYADDFKGRFAFSLETSASTAYLQINNLKNEDTATYFCALARWNRDAMDYWGQGTSVTVSS

SEQ ID NO: 7 - DNA, 339 bp, Mus sp.
gacattgtgctgacccaatctccagcttctttggctgtgtctcttgggcagagggccaccatatcctgcagagccagtgaaagtgttgatagttctgacaatagtcttatgcactggtaccagcagaaaccaggacagccacccaaactcctcatctatcgtgcatccaacctagaatctgggatccctgccaggttcagtggcagtgggtctaggacagacttcaccctcaccattaatcctgtggaggctgatgatgttgcaacctattactgtcagcaaagtattggggatcctccgtacacgttcggaggggggaccaagctggaaataaaacgg

SEQ ID NO: 8 - protein, 113 aa, Mus sp.
DIVLTQSPASLAVSLGQRATISCRASESVDSSDNSLMHWYQQKPGQPPKLLIYRASNLESGIPARFSGSGSRTDFTLTINPVEADDVATYYCQQSIGDPPYTFGGGTKLEIKR

SEQ ID NO: 9 - DNA, 363 bp, Mus sp.
caggtccaactgcagcagcctggggctgaacttgtgaagcctggggcttcagtgaagctgtcctgcaaggcttctggctacaccttcaccagctactggatgcactgggtgaagcagaggcctggacagggccttgagtggatcggagagattgatccttctgattcttatactaactacaatcagaagttcaagggcaaggccacattgactgtagacaaatcctccagcacagcctacatgcagctcagcagcctgacatctgaggactctgcggtctattactgtgcaagggggggtacaggagactttcactatgctatggactactggggtcaaggcacctcggtcaccgtatcatcg

SEQ ID NO: 10 - protein, 123 aa, Mus sp.
QVQLQQPGAELVKPGASVKLSCKASGYTFTSYWMHWVKQRPGQGLEWIGEIDPSDSYTNYNQKFKGKATLTVDKSSSTAYMQLSSLTSEDSAVYYCALARGGTGDFHYAMDYWGQGTSVTVSS

SEQ ID NO: 11 - DNA, 324 bp, Mus sp.
gacatcctgatgacccaatctccatcctccatgtctgtatctctgggagacacagtcagcatcacttgccatgcaagtcagggcattagcagtaatatagggtggttgcagcagaaaccagggaaatcatttaagggcctgatctatcatggaaccaacttggaagatggagttccatcaaggttcagtggcagtggatctggagcagattattctctcaccatcagcagcctggaatctgaagattttgcagactattactgtgtacagtatgttcagttcccgtacacgttcggagggggcaccaagctggaaatcaaacgg

SEQ ID NO: 12 - protein, 108 aa, Mus sp.
DILMTQSPSSMSVSLGDTVSITCHASQGISSNIGWLQQKPGKSFKGLIYHGTNLEDGVPSRFSGSGSGADYSLTISSLESEDFADYYCVQYVQFPYTFGGGTKLEIKR

SEQ ID NO: 13 - DNA, 21 bp, Unknown
  PCR primer
catgggatgctgccggtgtat

SEQ ID NO: 14 - DNA, 21 bp, Unknown
  PCR primer
aattctgggccttggctgacg

SEQ ID NO: 15 - DNA, 21 bp, Unknown
  PCR primer
tggccgcctctgtcgaagaag

SEQ ID NO: 16 - DNA, 363 bp, Mus sp.
caggtccaactgcagcagcctggggctgagcttgtgaagcctggggcttcagtgaacctgtcctgtaaggcttctggctacaccttcaccagctactggatgcactgggtgaagcagaggcctggacaaggccttgagtggatcggagagattgatccttctgatagttttactacctacaatcaaaacttcaaagacagggccacattgactgtagacaaatcatccagcacagcctacatgcagctcagaagtctgacatctgaggactctgcggtctattactgtgccagggggggtccaggagactttcgctatgctatggattactggggccaaggcacctcggtcaccgtctcctca

SEQ ID NO: 17 - protein, 123 aa, Mus sp.
QVQLQQPGAELVKPGASVNLSCKASGYTFTSYWMHWVKQRPGQGLEWIGEIDPSDSFTTYNQNFKDRATLTVDKSSSTAYMQLRSLTSEDSAVYYCALARGGPGDFRYAMDYWGQGTSVTVSS

SEQ ID NO: 18 - DNA, 327 bp, Mus sp.
gacatcctgatgacccaatctccatcctccatgtctgtatctctgggagacacagtcagcatcacttgccatgcaagtcagggcattagcagtaatatagggtggttgcagcagaaaccagggaaatcatttaagggcctgatctatcatggaaccaacttggaagatggagttccatcaaggttcagtggcagtggatctggagcagattattctctcaccatcagcagcctggaatccgaagactttgcagactattactgtgtacagtatgttcagtttccctacacgttcggaggggggaccaagctggaaataaaacgggct

SEQ ID NO: 19 - protein, 109 aa, Mus sp.
DILMTQSPSSMSVSLGDTVSITCHASQGISSNIGWLQQKPGKSFKGLIYHGTNLEDGVPSRFSGSGSGADYSLTISSLESEDFADYYCVQYVQFPYTFGGGTKLEIKRA

SEQ ID NO: 20 - DNA, 363 bp, Mus sp.
caggtccaactgcagcagtctggggctgagctggtgaggcctggggtctcagtgaagatttcctgcaagggttctggctacacattcactgattatgctatgcattgggtgaagcagagtcatgcaaagagtctagagtggattggacttattaatactgactatggtgatactacttacaaccagaagttcaagggcaaggccacaatgactgtagacaaatcctccaacacagcctatatggaacttgccagactgacatctgaggattctgccatctattactgtgcaagatcggactatgattactatttctgtggtatggactactggggtcaaggaaccacggtcaccgaatctcta

SEQ ID NO: 21 - protein, 125 aa, Mus sp.
QVQLQQSGAELVRPGVSVKISCKGSGYTFTDYALAMHWVKQSHAKSLEWIGLINTDYGDTTYNQKFKGKATMTVDKSSNTAYMELARLTSEDSAIYYCALARSDYDYYFCGMDYWGQGTTVTESL

SEQ ID NO: 22-23 - skipped (2 entries)

SEQ ID NO: 24 - DNA, 357 bp, Mus sp.
caggtgcagctgaaggagtcaggacctggcctggtggcgccctcacagcgcctgtccatcacatgcaccgtctcagggttctcattaaccggctatggtgtacactggattcgccagtctccaggaaagggtctggagtggctgggaatgatatgggctgagggaagaaccgactataattcagttctcaaatccagactgagcatcaataaggacaattccaggagccaagttttcttagaaatgaacagtctgcaaactgatgacacagccaggtactactgtgccagagaggtgattactacggaagcctggtacttcgatgtctggggccaaggaacctcggtcaccgaatct

SEQ ID NO: 25 - protein, 119 aa, Mus sp.
QVQLKESGPGLVAPSQRLSITCTVSGFSLTGYGVHWIRQSPGKGLEWLGMIWAEGRTDYNSVLKSRLSINKDNSRSQVFLEMNSLQTDDTARYYCAREVITTEAWYFDVWGQGTSVTES

SEQ ID NO: 26 - DNA, 324 bp, Mus sp.
gacattgtgatgactcagtctccagccaccctgtctgtgactccaggagatagagtctctctttcctgcagggccagccagagtattagcgactacttatactggtatcaacaaaaatcacatgagtctccaaggcttctcatcaaatatgcttcccaatccatctctgggatcccctccagattcagtggcagtggatcagggtcagatttcactctcactatcaacagtgtggaacctgaagatgttggaatgtattactgtcaaaatggtcacacctttccgctcacgttcggtgctggcaccaagctggaaatcaaacgg

SEQ ID NO: 27 - protein, 108 aa, Mus sp.
DIVMTQSPATLSVTPGDRVSLSCRASQSISDYLYWYQQKSHESPRLLIKYASQSISGIPSRFSGSGSGSDFTLTINSVEPEDVGMYYCQNGHTFPLTFGAGTKLEIKR

SEQ ID NO: 28 - protein, 8 aa, Mus sp.
GYTFSNYW

SEQ ID NO: 29 - protein, 8 aa, Mus sp.
ILPGSDRT

SEQ ID NO: 30 - protein, 13 aa, Mus sp.
ANRYDGYYFGLDY

SEQ ID NO: 31 - protein, 5 aa, Mus sp.
SSVNF

SEQ ID NO: 32 - protein, 3 aa, Mus sp.
YSS

SEQ ID NO: 33 - protein, 9 aa, Mus sp.
QHFTSSPYT

SEQ ID NO: 34 - protein, 8 aa, Mus sp.
GYSFTNYG

SEQ ID NO: 35 - protein, 8 aa, Mus sp.
INTHTGEP

SEQ ID NO: 36 - protein, 10 aa, Mus sp.
ARWNRDAMDY

SEQ ID NO: 37 - protein, 10 aa, Mus sp.
ESVDSSDNSL

SEQ ID NO: 38 - protein, 3 aa, Mus sp.
RAS

SEQ ID NO: 39 - protein, 10 aa, Mus sp.
QQSIGDPPYT

SEQ ID NO: 40 - protein, 8 aa, Mus sp.
GYTFTSYW

SEQ ID NO: 41 - protein, 8 aa, Mus sp.
IDPSDSYT

SEQ ID NO: 42 - protein, 14 aa, Mus sp.
ARGGTGDFHYAMDY

SEQ ID NO: 43 - protein, 6 aa, Mus sp.
QGISSN

SEQ ID NO: 44 - protein, 3 aa, Mus sp.
HGT

SEQ ID NO: 45 - protein, 8 aa, Mus sp.
QYVQFPYT

SEQ ID NO: 46 - protein, 8 aa, Mus sp.
GYTFTSYW

SEQ ID NO: 47 - protein, 8 aa, Mus sp.
IDPSDSFT

SEQ ID NO: 48 - protein, 14 aa, Mus sp.
ARGGPGDFRYAMDY

SEQ ID NO: 49 - protein, 6 aa, Mus sp.
QGISSN

SEQ ID NO: 50 - protein, 3 aa, Mus sp.
HGT

SEQ ID NO: 51 - protein, 9 aa, Mus sp.
VQYVQFPYT

SEQ ID NO: 52 - protein, 8 aa, Mus sp.
GYTFTDYA

SEQ ID NO: 53 - protein, 8 aa, Mus sp.
INTDYGDT

SEQ ID NO: 54 - protein, 14 aa, Mus sp.
ARSDYDYYFCGMDY

SEQ ID NO: 55-57 - skipped (3 entries)

SEQ ID NO: 58 - protein, 8 aa, Mus sp.
GFSLTGYG

SEQ ID NO: 59 - protein, 7 aa, Mus sp.
IWAEGRT

SEQ ID NO: 60 - protein, 14 aa, Mus sp.
AREVITTEAWYFDV

SEQ ID NO: 61 - protein, 6 aa, Mus sp.
QSISDY

SEQ ID NO: 62 - protein, 3 aa, Mus sp.
YAS

SEQ ID NO: 63 - protein, 9 aa, Mus sp.
QNGHTFPLT

SEQ ID NO: 64 - protein, 7 aa, Artificial Sequence
  Peptide that binds to monoclonal antibody 8H5 or 3C8
HGMLPVY

SEQ ID NO: 65 - protein, 7 aa, Artificial Sequence
  Peptide that binds to monoclonal antibody 8H5 or 3C8
PPSNYGR

SEQ ID NO: 66 - protein, 7 aa, Artificial Sequence
  Peptide that binds to monoclonal antibody 8H5 or 3C8
PPSNFGK

SEQ ID NO: 67 - protein, 7 aa, Artificial Sequence
  Peptide that binds to monoclonal antibody 8H5 or 3C8
GDPWFTS

SEQ ID NO: 68 - protein, 7 aa, Artificial Sequence
  Peptide that binds to monoclonal antibody 8H5 or 3C8
NSGPWLT

SEQ ID NO: 69 - skipped

SEQ ID NO: 70 - protein, 7 aa, Artificial Sequence
  Peptide that binds to monoclonal antibody 8H5 or 3C8
WPPLSKK

SEQ ID NO: 71 - protein, 7 aa, Artificial Sequence
  Peptide that binds to monoclonal antibody 8H5 or 3C8
NTFRTPI

SEQ ID NO: 72 - protein, 7 aa, Artificial Sequence
  Peptide that binds to monoclonal antibody 8H5 or 3C8
NTFRDPN

SEQ ID NO: 73 - protein, 7 aa, Artificial Sequence
  Peptide that binds to monoclonal antibody 8H5 or 3C8
NPIWTKL

SEQ ID NO: 74 - protein, 12 aa, Artificial Sequence
  Peptide that binds to monoclonal antibody 8H5
MEPVKKYPTRSP

SEQ ID NO: 75 - DNA, 36 bp, Artificial Sequence
  Peptide that binds to monoclonal antibody 8H5
atggagccggtgaagaagtatccgacgcgttctcct

SEQ ID NO: 76 - protein, 12 aa, Artificial Sequence
  Peptide that binds to monoclonal antibody 8H5
ETQLTTAGLRLL

SEQ ID NO: 77 - DNA, 36 bp, Artificial Sequence
  Peptide that binds to monoclonal antibody 8H5
gagactcagctgactacggcgggtcttcggctgctt

SEQ ID NO: 78 - protein, 12 aa, Artificial Sequence
  Peptide that binds to monoclonal antibody 8H5
ETPLTETALKWH

SEQ ID NO: 79 - DNA, 36 bp, Artificial Sequence
  Peptide that binds to monoclonal antibody 8H5
gagacgcctcttacggagacggctttgaagtggcat

SEQ ID NO: 80 - protein, 12 aa, Artificial Sequence
  Peptide that binds to monoclonal antibody 8H5
QTPLTMAALELF

SEQ ID NO: 81 - DNA, 36 bp, Artificial Sequence
  Peptide that binds to monoclonal antibody 8H5
cagacgccgctgactatggctgctcttgagcttttt

SEQ ID NO: 82 - protein, 12 aa, Artificial Sequence
  Peptide that binds to monoclonal antibody 8H5
DTPLTTAALRLV

SEQ ID NO: 83 - DNA, 36 bp, Artificial Sequence
  Peptide that binds to monoclonal antibody 8H5
gatactccgctgacgacggcggctcttcggctggtt

SEQ ID NO: 84 - protein, 12 aa, Artificial Sequence
  Peptide that binds to monoclonal antibody 8H5
TPLTLWALSGLR

SEQ ID NO: 85 - DNA, 36 bp, Artificial Sequence
  Peptide that binds to monoclonal antibody 8H5
acgccgcttacgctttgggctctttctgggctgagg

SEQ ID NO: 86 - protein, 12 aa, Artificial Sequence
  Peptide that binds to monoclonal antibody 8H5
QTPLTETALKWH

SEQ ID NO: 87 - DNA, 36 bp, Artificial Sequence
  Peptide that binds to monoclonal antibody 8H5
cagacgcctcttacggagacggctttgaagtggcat

SEQ ID NO: 88 - protein, 12 aa, Artificial Sequence
  Peptide that binds to monoclonal antibody 8H5
QTPLTMAALELL

SEQ ID NO: 89 - DNA, 36 bp, Artificial Sequence
  Peptide that binds to monoclonal antibody 8H5
cagacgcctctgactatggcggctcttgagcttctt

SEQ ID NO: 90 - protein, 12 aa, Artificial Sequence
  Peptide that binds to monoclonal antibody 8H5
HLQDGSPPSSPH

SEQ ID NO: 91 - DNA, 36 bp, Artificial Sequence; identical to SEQ ID NO: 89
  Peptide that binds to monoclonal antibody 8H5

SEQ ID NO: 92 - protein, 12 aa, Artificial Sequence
  Peptide that binds to monoclonal antibody 8H5
GHVTTLSLLSLR

SEQ ID NO: 93 - DNA, 36 bp, Artificial Sequence
  Peptide that binds to monoclonal antibody 8H5
gggcatgtgacgactctttctcttctgtcgctgcgg

SEQ ID NO: 94 - protein, 12 aa, Artificial Sequence
  Peptide that binds to monoclonal antibody 8H5
FPNFDWPLSPWT

SEQ ID NO: 95 - DNA, 36 bp, Artificial Sequence
  Peptide that binds to monoclonal antibody 8H5
tttccgaattttgattggcctctgtctccgtggacg

SEQ ID NO: 96 - protein, 12 aa, Artificial Sequence
  Peptide that binds to monoclonal antibody 8H5
ETPLTEPAFKRH

SEQ ID NO: 97 - DNA, 36 bp, Artificial Sequence
  Peptide that binds to monoclonal antibody 8H5
gagacgcctcttacggagccggcttttaagcggcat

SEQ ID NO: 98 - DNA, 10 bp, Artificial Sequence
  Pyranosyl RNA capture moiety partner
tagaacgaag

SEQ ID NO: 99 - DNA, 10 bp, Artificial Sequence
  Pyranosyl RNA capture moiety partner
cttcgttcta

SEQ ID NO: 100 - DNA, 10 bp, Artificial Sequence
  Pyranosyl RNA capture moiety partner
tcagtggatg

SEQ ID NO: 101 - DNA, 10 bp, Artificial Sequence
  Pyranosyl RNA capture moiety partner
catccactga

SEQ ID NO: 102 - DNA, 10 bp, Artificial Sequence
  Pyranosyl RNA capture moiety partner
gtattgcgag

SEQ ID NO: 103 - DNA, 10 bp, Artificial Sequence
  Pyranosyl RNA capture moiety partner
ctcgcaatac

SEQ ID NO: 104 - DNA, 8 bp, Artificial Sequence
  Pyranosyl RNA capture moiety partner
aacgattc

SEQ ID NO: 105 - DNA, 8 bp, Artificial Sequence
  Pyranosyl RNA capture moiety partner
gaatcgtt

SEQ ID NO: 106 - DNA, 8 bp, Artificial Sequence
  Pyranosyl RNA capture moiety partner
agtggatg

SEQ ID NO: 107 - DNA, 8 bp, Artificial Sequence
  Pyranosyl RNA capture moiety partner
catccact

SEQ ID NO: 108 - DNA, 8 bp, Artificial Sequence
  Pyranosyl RNA capture moiety partner
gtattgcg

SEQ ID NO: 109 - DNA, 8 bp, Artificial Sequence
  Pyranosyl RNA capture moiety partner
cgcaatac

SEQ ID NO: 110 - DNA, 8 bp, Artificial Sequence
  Pyranosyl RNA capture moiety partner
atgccttc

SEQ ID NO: 111 - DNA, 8 bp, Artificial Sequence
  Pyranosyl RNA capture moiety partner
gaaggcat

SEQ ID NO: 112 - DNA, 8 bp, Artificial Sequence
  Pyranosyl RNA capture moiety partner
tgatggac

SEQ ID NO: 113 - DNA, 8 bp, Artificial Sequence
  Pyranosyl RNA capture moiety partner
gtccatca

SEQ ID NO: 114 - DNA, 8 bp, Artificial Sequence
  Pyranosyl RNA capture moiety partner
cagtagtg

SEQ ID NO: 115 - DNA, 8 bp, Artificial Sequence
  Pyranosyl RNA capture moiety partner
cactactg

SEQ ID NO: 116 - DNA, 8 bp, Artificial Sequence
  Pyranosyl RNA capture moiety partner
ttcctgag

SEQ ID NO: 117 - DNA, 8 bp, Artificial Sequence
  Pyranosyl RNA capture moiety partner
ctcaggaa

SEQ ID NO: 118 - DNA, 8 bp, Artificial Sequence
  Pyranosyl RNA capture moiety partner
gactctct

SEQ ID NO: 119 - DNA, 8 bp, Artificial Sequence
  Pyranosyl RNA capture moiety partner
agagagtc

SEQ ID NO: 120 - DNA, 9 bp, Artificial Sequence
  Pyranosyl RNA capture moiety partner
atgcdcttc

SEQ ID NO: 121 - DNA, 8 bp, Artificial Sequence
  Pyranosyl RNA capture moiety partner
gaadgcat

SEQ ID NO: 122 - DNA, 9 bp, Artificial Sequence
  Pyranosyl RNA capture moiety partner
gtcdcatca

SEQ ID NO: 123 - DNA, 6 bp, Artificial Sequence
  Pyranosyl RNA capture moiety partner
cagtag

SEQ ID NO: 124 - DNA, 6 bp, Artificial Sequence
  Pyranosyl RNA capture moiety partner
ctactg

SEQ ID NO: 125 - DNA, 6 bp, Artificial Sequence
  Pyranosyl RNA capture moiety partner
gactct

SEQ ID NO: 126 - DNA, 6 bp, Artificial Sequence
  Pyranosyl RNA capture moiety partner
agagtc